SEQUENCE LISTING (27 sequences)

SEQ ID NO: 1 - DNA, 1014 bp, Artificial Sequence
  A synthetic codon optimized oligonucleotide
atgttattccaaaacgtttctatcgctggtttagctcacatcgatgctccacacactttaacttctaaagaaatcaacgaacgtttacaaccaacttacgatcgtttaggtatcaaaactgatgttttaggtgatgttgctggtatccacgctcgtcgtttatgggatcaagatgttcaagcttctgatgctgctactcaagctgctcgtaaagctttaatcgatgctaacatcggtatcgaaaaaatcggtttattaatcaacacttctgtttctcgtgattacttagaaccatctactgcttctatcgtttctggtaacttaggtgtttctgatcactgtatgactttcgatgttgctaacgcttgtttagctttcatcaacggtatggatatcgctgctcgtatgttagaacgtggtgaaatcgattacgctttagttgttgatggtgaaactgctaacttagtttacgaaaaaactttagaacgtatgacttctccagatgttactgaagaagaattccgtaacgaattagctgctttaactttaggttgtggtgctgctgctatggttatggctcgttctgaattagttccagatgctccacgttacaaaggtggtgttactcgttctgctactgaatggaacaaattatgtcgtggtaacttagatcgtatggttactgatactcgtttattattaatcgaaggtatcaaattagctcaaaaaactttcgttgctgctaaacaagttttaggttgggctgttgaagaattagatcaattcgttatccaccaagtttctcgtccacacactgctgctttcgttaaatctttcggtatcgatccagctaaagttatgactatcttcggtgaacacggtaacatcggtccagcttctgttccaatcgttttatctaaattaaaagaattaggtcgtttaaaaaaaggtgatcgtatcgctttattaggtatcggttctggtttaaactgttctatggctgaagttgtttgg

SEQ ID NO: 2 - DNA, 903 bp, Artificial Sequence
  A synthetic codon optimized oligonucleotide
atgacctacccgggttatagctttacgccgaaacgcctggacgtccgtccgggtattgcgatgagctacctggacgaaggtccgagcgatggcgaggtggtcgtcatgctgcacggcaacccgtcttggggctatctgtggcgtcatctggtgagcggtctgtccgatcgctaccgttgtatcgtaccggaccacatcggtatgggtctgtctgacaaaccggacgatgcgccggacgcacaaccacgttacgattatactctgcagagccgtgtggacgacctggaccgtctgttgcaacatttgggcattaccggtccgattaccttggcagtccacgactggggtggtatgattggcttcggctgggccctgagccatcacgcccaagttaagcgtctggttatcaccaacacggcagctttcccgctgccgccagagaaacctatgccgtggcagattgcgatgggtcgccattggcgtttgggcgagtggtttatccgcaccttcaacgctttcagctcgggtgcgtcttggctgggcgtcagccgtcgtatgcctgcggcagtgcgccgtgcgtatgttgccccatacgataattggaagaatcgtattagcacgatccgctttatgcaggatatcccgctgtccccggcagatcaggcgtggagcctgctggagcgtagcgcgcaagccctgccgtcctttgcagatcgtccggcattcatcgcttggggtctgcgcgatatttgctttgacaagcatttcctggcgggtttccgtcgtgcgttgccgcaggccgaagtgatggcgtttgacgatgcgaaccattacgttctggaagataaacatgaagttctggttccggccatccgcgcgttcctggagcgcaatccgctgtag

SEQ ID NO: 3 - DNA, 1650 bp, Artificial Sequence
  A synthetic codon optimized oligonucleotide
atgactaccctgtgcaacatcgccgcttccctgcctcgtttggcccgtgaacgcccagatcagattgcgatccgttgtccgggtggccgtggcgcgaacggcatggccgcatacgatgttaccctgagctacgcggaactggacgcacgttctgatgccattgcagccggtttggcgctgcatggtattggtcgtggcgttcgcgcggtcgtcatggtgcgcccgtccccggagttcttcctgttgatgttcgcactgttcaaagcgggtgcggtaccggttctggtcgatccgggtatcgacaagcgtgccctgaaacaatgtctggacgaggcacagcctcaggcgttcattggcattccgctggcgcagctggctcgtcgtctgctgcgctgggctccgtctgcgacccaaattgtgacggtcggtggtcgttattgttggggtggtgttacgctggcacgtgtcgagcgcgatggtgcaggtgcaggcagccaactggccgacacggcagcggacgacgtggctgcgattctgttcacgtcgggcagcaccggtgtgccgaaaggcgtggtttaccgtcaccgccactttgttggccaaatcgagctgctgcgtaatgccttcgacatgcagccgggtggcgtagacttgccgacgtttcctccgttcgcgttgtttgatccggcgctgggtctgaccagcgtcattccggacatggatccgacccgtccggctaccgcagacccgcgtaagctgcatgatgcgatgacgcgcttcggtgtgacccaattgttcggtagcccggcactgatgcgcgttctggcggactacggccaaccactgccgaatgttcgcctggcgacgagcgctggtgcgccggtgccgccagacgttgtcgccaaaattcgtgcactgctgccggctgatgcgcagttctggacgccgtatggcgctaccgaatgcctgccggttgcggcgatcgagggtcgtaccctggatgcgactcgcaccgcaaccgaagctggtgcgggtacctgcgtgggccaggtggttgcaccgaatgaggtccgtatcattgcgattgacgacgcggcgatcccggaatggagcggcgtgcgtgtgctggcggcaggtgaggtcggtgagatcacggtggcgggtccgaccaccacggatacctacttcaaccgtgatgcggcgacccgtaacgctaagatccgtgagcgttgcagcgatggtagcgaacgtgttgtgcaccgcatgggtgacgtgggctattttgacgcggaaggtcgtctgtggttttgtggccgtaagacccatcgcgttgaaactgcaaccggtccgctgtatacggagcaggtcgagccgatctttaacgtgcacccgcaggtccgccgtaccgcactggttggcgtgggcacgcctggtcagcaacagccggtcctgtgcgttgagttgcaaccgggcgttgccgcgagcgcatttgctgaggttgaaacggcgttgcgtgcagtcggtgcagcccatccacacaccgcgggtattgcccgttttctgcgccacagcggctttccggtggatatccgccacaatgccaagatcggtcgcgaaaaactggcgatctgggccgcacaacaacgtgtc

SEQ ID NO: 4 - DNA, 1008 bp, Artificial Sequence
  A synthetic codon optimized oligonucleotide
atgaaaatcctggttaccggtggtggtggttttctgggccaagccctgtgtcgtggtttggtcgcacgtggtcacgaggttgtcagctttcagcgcggtgactacccggtcctgcacacgttgggcgtgggccaaatccgtggtgacctggcagaccctcaggcggtccgtcacgctttggcaggtattgatgccgtttttcacaatgccgccaaagcgggtgcatggggcagctatgattcttatcatcaagcgaatgtcgttggtactcaaaatgtcctggatgcgtgtcgcgcgaacggcgtcccgcgtttgatctacacctccaccccgtcggtgacgcatcgtgcgacgaatccggttgagggtttgggtgcggatgaagttccgtacggtgaggacttgcgtgcgccgtacgctgcgaccaaggctatcgcggagcgtgcggtcctggcagccaacgacgcgcaattggcaaccgttgcgctgcgcccacgcctgatttggggtccgggtgacaatcacctgctgccgcgtctggcagcgcgtgcccgtgccggtcgcctgcgtatggtcggtgatggcagcaacctggtggactctacctatatcgataatgcagcccaggcccacttcgatgcgtttgcgcacctggcgcctggtgcagcttgcgcgggtaaggcatacttcattagcaacggcgaaccgctgccgatgcgtgagctgctgaaccgtctgctggcagcggtggatgccccagcggtgacccgtagcctgagcttcaaaaccgcgtaccgcatcggcgctgtgtgcgaaaccctgtggccgctgctgcgcctgccgggtgaggttccgctgacgcgtttcttggttgaacagctgtgcactccgcactggtacagcatggaaccagcacgtcgcgacttcggctatgttccgcagatttctatcgaggaaggcctgcagcgtttgcgttccagcagcagccgcgacattagcattacgcgc

SEQ ID NO: 5 - protein, 550 aa, Xanthomonas campestris
MTTLCNIAASLPRLARERPDQIAIRCPGGRGANGMAAYDVTLSYAELDARSDAIAAGLALHGIGRGVRAVVMVRPSPEFFLLMFALFKAGAVPVLVDPGIDKRALKQCLDEAQPQAFIGIPLAQLARRLLRWARSATQIVTVGGRYGWGGVTLARVERDGAGAGSQLADTAADDVAAILFTSGSTGVPKGVVYRHRHFVGQIELLRNAFDMQPGGVDLPTFPPFALFDPALGLTSVIPDMDPTRPATADPRKLHDAMTRFGVTQLFGSPALMRVLADYGQPLPNVRLATSAGAPVPPDVVAKIRALLPADAQFWTPYGATECLPVAAIEGRTLDATRTATEAGAGTCVGQVVAPNEVRIIAIDDAAIPEWSGVRVLAAGEVGEITVAGPTTTDTYFNRDAATRNAKIRERCSDGSERVVHRMGDVGYFDAEGRLWFCGRKTHRVETATGPLYTEQVEPIFNVHPQVRRAALVGVGTPGQQQPVLCVELQPGVAASAFAEVETALRAVGAAHPHTAGIARFLRHSGFPVDIRHNAKIGREKLAIWAAQQPR

SEQ ID NO: 6-8 - skipped (3 entries)

SEQ ID NO: 9 - DNA, 903 bp, Artificial Sequence
  A synthetic codon optimized oligonucleotide
atgacctacccgggttatagctttacgccgaaacgcctggacgtccgtccgggtattgcgatgagctacctggacgaaggtccgagcgatggcgaggtggtcgtcatgctgcacggcaacccgtcttggggctatctgtggcgtcatctggtgagcggtctgtccgatcgctaccgttgtatcgtaccggaccacatcggtatgggtctgtctgacaaaccggacgatgcgccggacgcacaaccacgttacgattatactctgcagagccgtgtggacgacctggaccgtctgttgcaacatttgggcattaccggtccgattaccttggcagtccacgcgtggggtggtatgattggcttcggctgggccctgagccatcacgcccaagttaagcgtctggttatcaccaacacggcagctttcccgctgccgccagagaaacctatgccgtggcagattgcgatgggtcgccattggcgtttgggcgagtggtttatccgcaccttcaacgctttcagctcgggtgcgtcttggctgggcgtcagccgtcgtatgcctgcggcagtgcgccgtgcgtatgttgccccatacgataattggaagaatcgtattagcacgatccgctttatgcaggatatcccgctgtccccggcagatcaggcgtggagcctgctggagcgtagcgcgcaagccctgccgtcctttgcagatcgtccggcattcatcgcttggggtctgcgcgatatttgctttgacaagcatttcctggcgggtttccgtcgtgcgttgccgcaggccgaagtgatggcgtttgacgatgcgaaccattacgttctggaagataaacatgaagttctggttccggccatccgcgcgttcctggagcgcaatccgctgtag

SEQ ID NO: 10 - DNA, 1656 bp, Artificial Sequence
  A synthetic codon optimized oligonucleotide
atgaatcgtccctgcaatattgcggctcgccttcccgagcttgctcgcgaacgccctgaccagatcgcgatccgttgccccggacgtcgcggtgccggaaacggcatggcagcttatgatgtgaccttggattaccgtcaattggacgcgcgtagcgacgcgatggcagcaggcctggctggatacggaattgggcgtggcgtccgtactgttgtcatggttcgtcccagccccgaatttttcctgttgatgttcgccttgtttaaattaggagcagttcctgttctggtcgatcctgggattgatcgccgcgcactgaagcaatgtttggacgaggctcagcctgaagcgtttatcggaattccactggcgcacgtagcccgtcttgttttacgttgggcgccatctgcggcccgtttagttacagtagggcgtcgtttgggctggggcggcactacgttggctgcacttgagcgcgctggggcgaagggcggtccaatgcttgcagcaaccgacggcgaggatatggctgccattttatttacctctgggtcaacaggagtaccgaagggggttgtgtatcgtcatcgccactttgtgggtcaaattcagcttttaggttctgcgttcgggatggaggctggaggagtcgacttgcctacatttccccccttcgctttattcgatcctgctctggggctgacctcggtaattcccgatatggacccaacgcgtcctgctcaggcagaccctgtccgcctgcatgacgctattcaacgcttcggagtcacacagcttttcggttcccctgcattaatgcgtgtactggctaaacatggtcgtccgttaccgacagtgacacgtgtaacgtcagccggagcacctgtacctcccgatgtagtagccacgattcgctcgttgttaccggcggatgcccagttttggactccgtacggggctacagagtgtttgcccgttgcagttgttgaagggcgtgaactggagcgtactcgcgctgcaactgaggcaggagcggggacatgcgttggaagtgtcgtagcaccgaacgaggtacgcatcatcgcgattgacgatgcgcctttagcagactggtcccaagcccgcgttctggctgttggcgaagttggggagattaccgtagcaggcccaactgctaccgatagctattttaatcgcccgcaagcaactgcagccgcaaaaatccgcgagacccttgcagatggttcgacgcgcgttgttcatcgtatgggcgatgtggggtactttgacgctcagggacgcttatggttctgcggtcgtaaaacccagcgcgttgagacggcgcgtgggccgctgtatacagagcaagtggagccagttttcaatactgtagcaggagttgcgcgtacggcactggtaggagttggcgcagctggagcccaagtaccagtgttatgtgtggagttgttgcgtgggcaaagcgatagtccagccttgcaagaagcgttacgcgcgcatgccgcagcacgcaccccggaggcgggtcttcaacattttctggtccatccagcgttccccgtcgacatccgtcacaacgccaagattgggcgtgaaaaattagccgtctgggcgtcggccgagttagagaaacgtgcc

SEQ ID NO: 11 - DNA, 1659 bp, Artificial Sequence
  A synthetic codon optimized oligonucleotide
atgtcggagcgctgtaacattgcggcggctctgccacgcttggcggcagaagcaccggatcgcgttgccatgcgttgtcctggaacgcatggggccaatggcctggcccgctatgacgttgccttaacgtatgctgggcttgatcgtcgttcagatgccattgccgcaggccttgccaaacacggggtcgcacgtggacaacgtgttgtcgttatggtgcgtccctccccggaattcttcctgttaatgttcgcgttatttaaggctggagccgtgcccgtccttgtcgaccccggcattgataagcgtgccttaaagcagtgtttagatgaggctcagccacacgcctttgtgggaattccacttgcgatgtttgcgcgcaagcttttaggctgggcgcgtggagcgaaggttgcggttacggtcggtcgccgttgggcgtggggaggtccaactctggcacaagtcgagcgtgacggcactggagcagggccgcagcttgccgatacagcaccagacgaagtggcggccatccttttcacctctggctcaacaggagtgcctaagggggttgtatatcgccaccgtcactttgtggcacaaatcgatatgcttcgtgacgcttttgggctgcaaccaggcggcgtagacctgccgacttttccaccatttgccctttttgaccctgcactggggttgtcgtcgattatccctgacatggacccgacacgcccagccaaagccgacccccgcaagctgcacgacgcgattgctcgcttcggagtagaccaattgtttggttcacccgctctgatgcgcgtgttggctgagtacggtcagccacttccgactttgcgccgtgtaactagcgcgggagcgcccgttccggcagatgttgttgctaagatgcgtgggttgttaccccccgaggcacaattctggaccccctacggggccacggaatgccttccagtcgccgtgatcgaggcacgcgaactgcaaagcacccgcgaagctacagaacaaggcgctggaacttgcgtaggacgcccagtccccccgaacgaggtacgtattattgcaatcaccgatgccccgattgcagattggagtcaagcgcagctgttgggtgctgaagcgattggtgaaattaccgtcgcaggccccagtgcgacggacgagtattttgctcgtccacaggcgactgctttagctaagatccgcgagacgctgcccgacggccgccagcgcatcgttcaccgtatgggagaccttggccgtttcgatgctcaagggcgcttgtggttctgcgggcgtaaaagccatcgcgttcgcaccccattgggtaacctttatacggagcaagtagaacctgttttcaacacacatccggaggttgcacgcacggccttggtcggcgttggagaaggcgcggcgcaagagccggtgctgtgtgtcgaaatggctccgcacctgcctcaatacgaacacgaacgtgtattagcagaactgcgccgcatgtccgaaggattcgtacatactgcgcgcatccgccatttccttgttcatgatgggttccctgtggacattcgccataacgcgaaaattgggcgcgagcaattggcagcttgggccgctaaagagttgcgctggcgtcgt

SEQ ID NO: 12 - DNA, 1641 bp, Artificial Sequence
  A synthetic codon optimized oligonucleotide
atgagtgcggcgtgtaacattgccgcaagtctgcctgcactggcgcgtgcgcgcggtgaacaggtagcgatgcgctgcccgggacgcgacggtcgttacgatgtggcgatcacttatgctgatttagatcgtcgttcagatgcgattgcagcgggtttgggtaagcgtggtattgtacgcgggactcgcaccgtggttatggtccgccccacacctgagttttttcttttgatgtttgctctgtttaaagcaggagctgttcctgtgttagtagaccccgggatcgacaaacgcgccttaaagcgttgcttagacgaggccgaaccggatgctttcattgggattcccctggcccattttgcgcgcacgttgctgggttgggctcgctccgcacgcattcgtgtgactacagggcgtcgcgcacttttaagcgacgctacgcttgccgatgttgagcgtgatggtgcaaacgccggtcctcaattagcggatacgcagccagatgacatcgcggccattttattcacctctggtagcaccggggtccctaaaggagtcgtctaccgccaccgccatttcgttgcgcaggtagaaatgctgcgcgacgcgttcgggctggccccaggaggcgtagacttaccgacttttccgcccttcgctcttttcgatccggcattgggagtgaccagtattatcccagatatggatccaacacgcccagcgcaggccgatccacgtcgcttgcttcaggcgattgagcgttttggagtaacccaattatttggttcacccgcgttagtgggtgtgttagcacgccatggggcacacttacccacggtaaaacgcgtgctgagtgctggggctcccgttccggcagacgtagtggcacgtatgcgcgatttgcttcctggtgatgctcaattgtggacgccgtatggagcgaccgaatgcctgcctgtgtcagtgattgagggtcgcgaattgcaatccacccgtgaggcgaccgagcgtggagcaggaacgtgcgtcggtctgccggtagctccaaatgaagtccgcatcattcgcattgacgatgatgctatcgctcagtggtcagatgcacttttggtcaagcaaggacaaattggagaaatcacggtggccgggcccactgcaactgacgcgtactttcgtcgtgatgacgccacccgcctggctaagattcgtgaagcgactcccgacggggagcgtattgtgcaccgcatgggcgatttggggtggatcgacggcgaaggacgcctgtggttctgcggccgtaagactcaccgcgtagtcatggcagacgggaccacactttacactgaacaggtggaaccaatttttaacgctgcattccgcggtatgcgtaccgctttggttggagtgggtccgaaaggtgctcagcgtccagttttatgttacgaggtgcctaaagacgtcggacacaatgctgctgatctgcctggggaattgcgccattttgccgaaggacgcgtgcacactgcgaaaattcaccattttttgccccaccctgggttcccggtagacatccgtcataacgcgaaaattgggcgcgagaaattagcagcgtgggcgacgcgccaattagaaaaacgcgca

SEQ ID NO: 13 - DNA, 2936 bp, Artificial Sequence
  A synthetic codon optimized oligonucleotide
atgccgcagattccagccgctccagccgcccttccacctgccgatcgtctgccgggttgggacccagcttggagccgtctggtcgaaatccgttccgcagcggatccggaaggtaccgtccgtacgctgcatgtcgccgataccggtccggtcctggcggcagcgggtgcagagattgttggtacgatcgttgcagttcatggtaatccgacgtggtcttggctgtggcgcagcctgctggcagagactgtccgtcgtgcgcgtcgtggtatggcggcttggcgtgtcgttgcgccggatcagctggacatgggtttctccgaacgtctggcgcacgctggtagccctagcgcagcatcgatgggccgtgcgggtgacacgtatcgtaccctgggtggccgcatcgcagatctggacgcactgctgactgccctgggtctgcgcgatctggccgcgaccggtcatccactgatcaccctgggccacgactggggcggtgttgttagcctgggttgggcagctcgtcatccggagctggtcgcgggtgtggcgacgctgaacaccgcggtccaccaaccggaaggtgcgccaattccggcaccgctgcaagcagcgttggcgggtcctgtgctgccggcatccacggttaccaccgacgcatttctgtccgtcaccacctcgctggccaccccggctttggaccgtgaaacccgtgccgcttaccatctgccgtacgacacggcggcacgtcgtggcggcgttggtggttttgtcgcagacattccggcggaccctggccacggtagccacccggagctgcagcgcgttggtgaagatctggcggcactgggtcgtaccgacgttccagcgctgattctgtggggtgctgacgacccggtttttctggaccgctacttggacgatctgcgtgatcgcctgccgcatgcccgtgtccaccgttatgagcgcgcaggccatctgctggttgacgaccgcgatatcaccgctccgctgctgcaatgggcgcagttgctgcgcggtggtcaattgtctgacccagcatcgggtttgccgggtccggtgcctcacgcgactgccgatgcagccgcagatccgggtctggaagtggacctgggcgaggacccgggtgcccgtgagccgggtgttgttcgtttgtgggatcacttgcgtgattggggtgcgccaggcagcgatcaccgtgagtatacggcgctggtggatatggcgggtgcgcaggctggccgcagcttggtcggcaccgcacgccgtccggtagcggtcacgtggggtgagctgcaagaaatggtttccgcgattgcaaccggcctgtgggctgctggtatgcgtccgggcgaccgtgtggctatgctggttccgcctggtcgtgatctgagcgcggcattgtacgcagtgctgcgcgttggcgccgtcgctgttgttgcggatcaaggtctgggtgtgaaaggtatgacccgtgcgatgaagagcgcacgtcctcgctggattattggtcgcacgccgggtctgacgctggctcgtgcgcaatcgtggcctggcacgcgtatcagcgtgaccgagccaggtgcggcgcagcgccgtctgctggacgtgagcgacagcctgtatgcaatggttgaccgtcatcgcgatccggcagcaggcgatgcggtcgacgagcatggtacggtcctgcctgagccggcactggatgcagatgcggcagtcctgttcacgagcggttctacgggtccggccaagggtgtggtgtacactcacgagcgtttgggccgcttggttgcactgatcagccgcaccctgggtatccgtccgggtggtagcctgctggccggtttcgcaccgttcgcgctgttgggcccagcactgggtgccgcgtccgttagcccggacatggatgtgacccaaccggcaaccctgacggcccaaaagctggccgacgcggccattgcgggtcaaagcagcgtgctgtttgctagcccggcagcgctggcaaacgtggtggcaactgcagacggtctggatgcaccgcagcgtgaggcgttggacgcggtgcgtctggtgctgagcgccggtgcaccggttcacccgcagctgatgcgccaagttagcgacctgatgccgaacgcgcgtgtccacaccccgtggggcatgaccgaaggtctgctgctgaccgatatcgatggtgatgaagtccagcgcctgcgtacggccgatgatgcgggcgtctgcgtgggtagcgcgctgccgacggtgtctctggcgatcgcaccgctgttggaagatggtagcgcggaagatgtcattctggatccggcacgcggtcacggcgtcttgggcgagattgtcgttagcgcaccgcacctgaaggaccgttacgacgcgctgtggcatacggaccagcagagcaagcgtgacggtctgtggcgccgtgatggccgtgtgtggcaccgtacggcggatgttggtcatttcgatgccgaaggtcgtgtttggctggaaggtcgcctgcagcacgtgatcaccacgccggaaggtcctgtcggtcctggtggtccggagaaaaccgttgatgcgctgggtccggttcgtcgtagcgccgttgtcggtgttggccctcgcggtacccaagcggttgttgtcgttgttgaagcagcagttccggctacccgtccggctcgtcgtcctggtcaccatcgcgatggccgtccgaaacagggcttggcgccgaccgccttggcatcggcggtgcgtgctgcgctggagccgctgccggtcgctgcggttttggttgctgacgagattccgaccgacattcgtcacaattctaaaatcgaccgtgcccgtgttgcagattgggccgaagcggttctggccggtggcaaagttggtgcgctgca

SEQ ID NO: 14 - DNA, 2936 bp, Artificial Sequence
  A synthetic codon optimized oligonucleotide
atgccgcagattccagccgctccagccgcccttccacctgccgatcgtctgccgggttgggacccagcttggagccgtctggtcgaaatccgttccgcagcggatccggaaggtaccgtccgtacgctgcatgtcgccgataccggtccggtcctggcggcagcgggtgcagagattgttggtacgatcgttgcagttcatggtaatccgacgtggtcttggctgtggcgcagcctgctggcagagactgtccgtcgtgcgcgtcgtggtatggcggcttggcgtgtcgttgcgccggatcagctggacatgggtttctccgaacgtctggcgcacgctggtagccctagcgcagcatcgatgggccgtgcgggtgacacgtatcgtaccctgggtggccgcatcgcagatctggacgcactgctgactgccctgggtctgcgcgatctggccgcgaccggtcatccactgatcaccctgggccacgcgtggggcggtgttgttagcctgggttgggcagctcgtcatccggagctggtcgcgggtgtggcgacgctgaacaccgcggtccaccaaccggaaggtgcgccaattccggcaccgctgcaagcagcgttggcgggtcctgtgctgccggcatccacggttaccaccgacgcatttctgtccgtcaccacctcgctggccaccccggctttggaccgtgaaacccgtgccgcttaccatctgccgtacgacacggcggcacgtcgtggcggcgttggtggttttgtcgcagacattccggcggaccctggccacggtagccacccggagctgcagcgcgttggtgaagatctggcggcactgggtcgtaccgacgttccagcgctgattctgtggggtgctgacgacccggtttttctggaccgctacttggacgatctgcgtgatcgcctgccgcatgcccgtgtccaccgttatgagcgcgcaggccatctgctggttgacgaccgcgatatcaccgctccgctgctgcaatgggcgcagttgctgcgcggtggtcaattgtctgacccagcatcgggtttgccgggtccggtgcctcacgcgactgccgatgcagccgcagatccgggtctggaagtggacctgggcgaggacccgggtgcccgtgagccgggtgttgttcgtttgtgggatcacttgcgtgattggggtgcgccaggcagcgatcaccgtgagtatacggcgctggtggatatggcgggtgcgcaggctggccgcagcttggtcggcaccgcacgccgtccggtagcggtcacgtggggtgagctgcaagaaatggtttccgcgattgcaaccggcctgtgggctgctggtatgcgtccgggcgaccgtgtggctatgctggttccgcctggtcgtgatctgagcgcggcattgtacgcagtgctgcgcgttggcgccgtcgctgttgttgcggatcaaggtctgggtgtgaaaggtatgacccgtgcgatgaagagcgcacgtcctcgctggattattggtcgcacgccgggtctgacgctggctcgtgcgcaatcgtggcctggcacgcgtatcagcgtgaccgagccaggtgcggcgcagcgccgtctgctggacgtgagcgacagcctgtatgcaatggttgaccgtcatcgcgatccggcagcaggcgatgcggtcgacgagcatggtacggtcctgcctgagccggcactggatgcagatgcggcagtcctgttcacgagcggttctacgggtccggccaagggtgtggtgtacactcacgagcgtttgggccgcttggttgcactgatcagccgcaccctgggtatccgtccgggtggtagcctgctggccggtttcgcaccgttcgcgctgttgggcccagcactgggtgccgcgtccgttagcccggacatggatgtgacccaaccggcaaccctgacggcccaaaagctggccgacgcggccattgcgggtcaaagcagcgtgctgtttgctagcccggcagcgctggcaaacgtggtggcaactgcagacggtctggatgcaccgcagcgtgaggcgttggacgcggtgcgtctggtgctgagcgccggtgcaccggttcacccgcagctgatgcgccaagttagcgacctgatgccgaacgcgcgtgtccacaccccgtggggcatgaccgaaggtctgctgctgaccgatatcgatggtgatgaagtccagcgcctgcgtacggccgatgatgcgggcgtctgcgtgggtagcgcgctgccgacggtgtctctggcgatcgcaccgctgttggaagatggtagcgcggaagatgtcattctggatccggcacgcggtcacggcgtcttgggcgagattgtcgttagcgcaccgcacctgaaggaccgttacgacgcgctgtggcatacggaccagcagagcaagcgtgacggtctgtggcgccgtgatggccgtgtgtggcaccgtacggcggatgttggtcatttcgatgccgaaggtcgtgtttggctggaaggtcgcctgcagcacgtgatcaccacgccggaaggtcctgtcggtcctggtggtccggagaaaaccgttgatgcgctgggtccggttcgtcgtagcgccgttgtcggtgttggccctcgcggtacccaagcggttgttgtcgttgttgaagcagcagttccggctacccgtccggctcgtcgtcctggtcaccatcgcgatggccgtccgaaacagggcttggcgccgaccgccttggcatcggcggtgcgtgctgcgctggagccgctgccggtcgctgcggttttggttgctgacgagattccgaccgacattcgtcacaattctaaaatcgaccgtgcccgtgttgcagattgggccgaagcggttctggccggtggcaaagttggtgcgctgca

SEQ ID NO: 15 - protein, 374 aa, Streptomyces toxytricini
MSTTERRSRIEALGAFLPAGRETNDELRAKVPNLGDADVRRITGIAERRVHDPDPAAGEDSFGMALAAARDCLAVSRHRAADLDVVISASITRVKDGSRFHFEPSFAGMLAKELGARPAISFDVSNACAGMMTGVWLLDRMIRSGAVRSGMVVSGEQATRVARTAARELRDSYDPQFASLSVGDSAAAVVLDESTDPADRIHYIELMTCAAYSHLCLGMPSDRSQGIGLYTDNKKMHDRERLKLWPRFHEDFLAKNGRRFEDEEFDHIIQHQVGTRFIEYANRTAEAEFAAPMPPSLQVVEQYGNTATTSHFLTLRDHLRRTRGAGATGTGTGPGSGPGAGPAREAAGAKYLLVPAASGLVTGALSATVTHAGA

SEQ ID NO: 16 - protein, 563 aa, Streptomyces toxytricini
MKILITGATGFLGGHLADACLRSGHGVRALVRPGSNTDRLRALPGVELVTGDLTRPDSLRRAADGCEAVLHSAARVVDHGTRAQFTEANVTGTLRLMDAARAAGVRRFVFVSSPSALMHLREGDRLGIDETTPYPTRWFNDYCATKAVAEQHVLAADTAGFTTCALRPRGIWGPRDHAGFLPRLIGALHAGRLPDLSGGKHVLVSLCHVDNAVDACLRAAVSAPAERIGGRAYFVADAETTDLWPFLADVAARLGCPPPAPRIPLPAGRALAAAVETAWRLRPDAAARARSSPPLSRYMMALLTRSSTYDTTAARRDLGYTPVRTQEDGLRDLVRWVASQGGVASWTAPRPHPAHTHTPDATPHAPARAPHPPMPEPPAAATPAPPPKAEHRPALPRPRSSPEADSTEQPFPHPADATDTPPVSGPAPGPVSVPAPDRTPAPSGSSRTAGDAPACRAGQASGPAPAPVRGPADARSAATGRGPRPVRGSAEQREHRDPSLRASGKPGSDGSGAPADTRPNHDPTRAEAARPGDAGRGMAPEGDTARRGSTDPAGPAGREDTSR

SEQ ID NO: 17 - protein, 491 aa, Kitasatospora cystarginea
MLYEALRDIAARRPDARAVTTADGASASYAELLDLIDRTAAGLRGHGVGAGDVIACSLRNSIRYVALILAAARIGARYVPLMSNFDRADIATALRLTGPRMIVTDHQREFPDQAPPRVRLETLEAATASPREAGERYDGLFRSLWTSGSTGFPKQMVWRQDRFLRERRRWLADTGITADDVFFCRHTLDVAHATDLHVFAALLSGAELVLADPDAAPDVLLRQIAERRATAMSALPRHYEEYVRAAAGRPAPDLSRLRRPLCGGAYVSAAQLTDAAEVLGIHIRQIYGSTEFGLAMGNMSDVLQAGVGMVPVEGVGVRLEPLAADRPDLGELVLISDCTSEGYVGSDEANARTFRGEEFWTGDVAQRGPDGTLRVLGRVTETLAAAGGPLLAPVLDEEIAAGCPVLETAALPAHPDRYSDEVLLVLHPDPDRPEQELRKAVAEVLDRHGLRASIRLTDDIPHTPVGKPDKPALRRRWESGALGPVGEWHHG

SEQ ID NO: 18 - protein, 491 aa, Streptomyces sp
MTALHAAVHEIARRRPDAIAVETTAGERTTYAELLARADRIAAGLRARGVTEGRVVVCSGLANDASYLAFLLGLCANGAAYVPLLADFDATAVDRALRMTRPVLWVGPDNHHRAGVTLPRVELADLETPAPATAPAAGGRALAPGTFRMLWTSGSTKAPKLVTWRQEPFVRERRRWIAHIEATERDAFFCRHTLDVAHATDLHAFAALLAGARLILADPAADPATLLAQLAATGATYTSMLPNHYEDLIAAARQRPGTDLSRLRRPMCGGAYASPALIADAADVLGIHIRHIYGSTEFGLALGNMADEVQTVGGMHEVAGVRARLEPLAGYDGDDLGHLVLTSDCTSDGYLDDDEANAATFRGPDFWTGDVARRLDDGSLRLLGRVTDLVLTTDGPLAAPHVDELVARHCPVAESVTLAADPDTLGNRVLVVLRAAPGTSDADAVGAVDKLLDAHGLTGVVLAFDRIPRTVVGKADRALLRRRHLPAPSSS

SEQ ID NO: 19 - protein, 719 aa, Streptomyces virginiae
MDQPAIETDSVAGWLERNARAFPDKPAVIHPDSRGSDGYRTITYGELQRTVEDLARGFRSAGITQGTRTVLMAPPGPELFALCFALFRVGAVPVVVDPGMGVRRMLHCYRAVGAEAFIGPPLAQLVRVLGRRTFAAVRVPVTLGRRRLGRGHTLTALRTAPATGRRADAAAPTGGDDLLMIGFTTGSTGPAKGVEYTHRMALSIARQIEEVHGRTRDDVSLVTLPFYGVLDLVYGSTLVLAPLAPARVAQADPALLVDALERFRVTTMFASPALLRNLAGHLTGSARGRHPLPDLRCVVSGGAPVPDTVVAALRRVLDEKAKIHVTYGATEVLPITSIEAAEILGDDDVRTDREDADAEGAEAEGAEAGSEAEAGSEAEAEAEAGSVALAASGAGTAARSAAGEGTCVGRPVPGTRVTIVPVTDGPLARLDSTTGLPAGRVGEILVHGDSVSRRYHRAPQSDAAHKVTEERPDGEDSRIWHRTGDLGHLDAEGRLWFCGRAVQRVRTGYRDLHTVRCEGVFNAHPLVRRTALVGIGPAGAQRPVVCVEIETGTGTGTGRGGGGGDGGAALDESGWTELVAELRTMAEAHAATTGLHEFLRHPGFPVDIRHNAKIGREELARWAARQQARSASSPARRAARIVPLAGWAYLVGGAVWAATGSAPDVPVLRWLWWIDAFLSIGVHAAQIPLALPRGRAAGHGTAAVVGRTMLYGATWWRAL

SEQ ID NO: 20 - protein, 874 aa, Streptomyces toxytricini
MATTTATPAAARPAAADDLGAHSLAGLLERNARAFPDKPAVIHPAAGPRRDGASPAYRTLTYGRLQQAVEELAAGLTRAGITKGTKTVLMAPPGPELFALAFALFRVGAVPVVVDPGMGVRRMLHCYRTVGAEAFIGPPLAHAARLLGRRAFAGIRVPVTLGRHRLGRARTLAAVRALGARGGAAAPVAAGRDDLLMIGFTTGSTGPAKGVEYTHRMALSAARQIEAVHGRTRDDTSLVTLPFYGVLDLVYGSTLVLAPLAPSRVAQADPALVVDALERFRVTTMFASPALLGPLAAHLAAAAPGRHPLPDLRCVVGGGAPVPDTTVAALRRALDPRARIHVTYGATEALPITSIEAEELLGPEDGGEGGGSGVGGAGSGGTAARAAEGAGTCVGRPVPGIGLAVLPVTDGPLTGSVPHLPTGRVGEIAVRGDCVSPRYHHSPDADRLHKVPDDTDPAGPAWHRTGDLGYLDDDGRLWFCGRSAQRVRTGTGDLHTVRCEGVFNAHPQVRRTALVGIPASPDSGWGRGGRTTTRSGTGSGGTGTARGATESSVAAGNGNTSTAAAPTTATDNGPAHSATPPCETTGNGTPRRPTPARVSAVSAPAHSATTVSGSSGRAAAVSGSAASAAPGSETVVGGSAGSTSAPGATTAGARAGSAAAGMAAEGSGTARSRTGGRGSAGDGTALGGSATAAPPGVAPGGVPADPRRNRLRPVVCVETVDEDLDEAAWQRLTAELRTLARTHAPTTDLQEFLHHPGFPVDIRHNAKIGREELARWAERRLTPPTPLTPRQRAARIVPLAGWAYLVGGAVWAAAFGVPEARLPRLLWWADAVLSTAGHAVQIPLALPRARTAGIGRPAAVGLTMLYGATWWRQL

SEQ ID NO: 21 - protein, 565 aa, Streptomyces aburaviensis
MMAASPRHPFEAEAGLADYLERHARTSPEKTAIIHPDGREADGGIRYRELSYGELQGRVEELAAGFSRIGITSGMRTILMPKPGPDLYILVFALLRIGAVPVVVDPGMGIKRMLNCYRAVGAEAFVGPSVAHAVRVLGRRTFSTVRIKVTLGRRWFWGGHTRDGLLGGSGSAPAGPVTGDDLMMIAFTTGSTGAAKGVESVHRMATATARQMHAAHGRDREDVSLVTVPIWGLFDLIYGSTMVLAPIAPAKVAQADPELLTAALTRFGVSTVFGSPALFRVLAAHLERERTPLPALRSVVSAGAPVPPDLVASLRRVLDERTGIHVAYGATEAMPISSIESAEILGETAARGALGDGTCVGRPVDGTDVRIVRVSDDPLPDWEAGLAVAPGEIGEIVVSGDVVSPRYHATADANAQYKIRERPAAGPERSWHRTGDLGYLDDAGRLWFCGRRAQRVRTAEGDLHTVRCEGVFNAHPLVRRSALVGIGAPGAQRPVVCVETEPGVGEEQWQELLTELRRLGAGRPLTAGLQEFLRHPGFPVDIRHNAKIGREELAGWAEQQTSART

SEQ ID NO: 22 - protein, 349 aa, Nocardia brasiliensis
MSKVLVTGASGFLGGALVRRLIRDGAHDVSILVRRTSNLADLGPDVDKVELVYGDLTDAASLVQATSGVDIVFHSAARVDERGTREQFWQENVRATELLLDAARRGGASAFVFISSPSALMDYDGGDQLDIDESVPYPRRYLNLYSETKAAAERAVLAADTTGFRTCALRPRAIWGAGDRSGPIVRLLGRTGTGKLPDISFGRDVYASLCHVDNIVDACVKAAANPATVGGKAYFIADAEKTNVWEFLGAVATRLGYEPPSRKPNPKVIDAVVGVIETIWRIPAVATRWSPPLSRYAVALMTRSATYDTGAAARDFGYQPVVDRETGLATFLAWLEKQGGAVELTRTLR

SEQ ID NO: 23 - protein, 342 aa, Thermobifida halotolerans
MRVLVTGASGFLGSHVAEACLRAGDEVRALVRPTSDPGHLRTLPGVEIVHDLGDTASLRAAAEGVDVVHHSAARVLDHGSRAQFWDTNVEGTRRLLEAARDGGARRFVFVSSPSAVMDGRDQVDVDESIPYPRRYLNLYSQTKAAAERLVLAADAPGFTTCALRPRAVWGPRDRHGFMPKLLGRLLAGRLPDLSGGRRVTAALCHCANAAHACVLAARADGVGGRAYFVTDAEPVDVWAFMAEVAEMFGAPPPRRRVPPVLRDALVEAVELAWRMPFLAHHHDPPLSRYSVALLTRSSTYDTAAARRDLGYRPLVDRSTGLEGLRSWVEEIGGPGVWTEGAR

SEQ ID NO: 24 - protein, 343 aa, Krasilnikovia cinnamomea
MKILVTGASGFLGGHIAEAAVAADHDVRALLRPTAALSMDAGADRVEPVRGDLTDPASLAVATAGVDVVIHSAARVTDHGSPAQFHDTNVAGTQRLLAAARANGVSRFVFVSSPSAVMDGTDQVGIDESTPYPAKYLNLYSETKAAAERLVLAANEPGFTTSALRPRGIWGPRDWHGFMPRLIAKLRAGRLPDLSGGRTVLASLCHATNAAHACLLAAGSDRVGGRAYFVADAEVSDVWALIAEVGAMFGAAPPTRRVPPAVRDALVATIETVWRVPYLRDRYSPPLSRYSVALLTRSSTYDTSAAARDFGYAPLLDQPTGLRQLREWVDGIGGVDAFTRYVR

SEQ ID NO: 25 - skipped

SEQ ID NO: 26 - protein, 288 aa, Streptomyces toxytricini
MGIVITASATATHTDPGTPASAVDLAGRAARRCLAHARVSPSGVGVLVNVGVYRENNTFEPALAALVQKETGINPDYLADPQPAAGFSFDLMDGACGVLSAVQAGQSLLSTGTTERLLITAADVHPGGDASRDPDYPYADLAGAFLLERDADPDTGFGPVRHYGGGDRPTDVAGYLDLDTMGSGGRSRITVHRTPGHEQRTGELAAAAVAAYTGEFGLDAGRTLVIGPDAPAGVGDGPGGGRPHTAAPVLGYLHALESARPEGVDTLLFVTAGAGPRAAVASYRPQGW

SEQ ID NO: 27 - protein, 557 aa, Nocardia brasiliensis
MSSATYWQAIDRFRAFARAEPDREAVIYPVGTDAAGLPAYRHISYRELDDWSETIAERLTASGVGSGTRTIVLVLPSPELYAILFALLKIGAVPVVIDPGMGLRKMVHCLRAVEAEAFIGIPPAHAVRVLFRRSFRKVRTTVTVGKRWFWRGAKLAAWGTTPSGGAVDRVPADPGDVLVIGFTTGSTGPAKAVELTHGNLASMIDQVHTARGEIAPETSLITLPLVGILDLLLGSRCVLPPLIPSKVGSTDPAHVAHAIETFGVRTMFASPALLIPLLRHLEQQPNELKTLASIYSGGAPVPDWCIAGLRAALTDDVQIFAGYGSTEALPMSLIESRELFDGLVERTHRGEGTCIGRPADRIDARIVAITDDPIPTWARAEELAGDLARSRGIGELVVAGPNVSTHYYWPDTANRQGKIVDGDRIWHRTGDLAWIDDAGRIWFCGRKSQRVVTADGPMFTVQVEQIFNTVAGVARTALVGVGAPGAQRPVLCIELKPDAEGAAVGAALRARGAEFDLSRPIADFLIHPGFPVDIRHNAKIGREQLAQWAGEQLGARA

(19) United States
(12) Patent Application Publication
(10) Pub. No.: US 2020/0048668 A1
(43) Pub. Date: Feb. 13, 2020

(54) BIOLOGICAL PRODUCTION OF ß-LACTONES

(71) Applicant: Regents of the University of Minnesota, Minneapolis, MN (US)

(72) Inventors: Serina L. Robinson, Wayzata, MN (US); James K. Christenson, Minneapolis, MN (US); Lawrence P. Wackett, St. Paul, MN (US)

(21) Appl. No.: 16/510,298

(22) Filed: Jul. 12, 2019

Related U.S. Application Data
(60) Provisional application No. 62/698,051, filed on Jul. 14, 2018.

(51) Int. Cl. C12P 17/02; C12Q 1/25

(52) U.S. Cl. CPC C12P 17/02; C12Q 1/25

(57) ABSTRACT

Methods of using β-lactone biosynthetic enzyme genes and host cells expressing one or more of those genes, e.g., heterologous expression, are provided.

### CROSS-REFERENCE TO RELATED APPLICATIONS

This application claims the benefit of the filing date of U.S. application Ser. No. 62/698,051, filed on Jul. 14, 2018. the disclosure of which is incorporated by reference herein.

### BACKGROUND

β-Lactones have been identified as important bacterial natural products over the last three decades, and include antibiotics, anti-cancer agents, and the only FDA-approved anti-obesity drug (tetrahydrolipstatin marketed as Orlistat, or Xenical). The tour-membered β-lactone rims is very reactive and can acylate active site nucleophiles of proteases, lipases and esterases. For example, the fatty acid-derived β-lactone natural product lipstatin from Streptomyces acts by inhibiting human pancreatic lipase thereby preventing the proper assimilation of fats from the diet. Another example is salinosporamide, a bicyclic β-lactone produced by Salinispora tropica that is known to inhibit human 20S protease function. Salinosporamide is now in phase III clinical trials for newly diagnosed glioblastoma and multiple myeloma and acts by inhibiting the tumor cells ability to degrade pro-apoptotic proteins. Synthetic β-lactones such as 3-benzyl, 4-propyl oxetanone are known to inhibit the ClpP protease of Mycobacterium tuberculosis. These results are especially exciting as proteases represent novel targets for antibiotics, suggesting β-lactones could provide an option for treating β-lactam resistant organisms.

However, only about 30 core scaffolds containing β-lactone moieties have been discovered in soil bacteria in the past 6 decades and a limited number have been synthesized by chemists through arduous procedures.

### SUMMARY

The disclosure provides methods of making β-lactones by employing a plurality of biosynthetic enzymes, e.g., OleA, OleB, OleC or OleD, or one of those enzymes, e.g., OleC or OleB, or homologs of those including but not limited to homologs of OleD such as NltD or LstD, and compounds prepared by the methods. The methods allow for synthesis of a large number of β-lactones. Also provided are computer methods to identify β-lactone producing genes in bacterial genomes. The use of the biosynthetic enzymes optionally in combination with other related enzymes, e.g., from heterologous sources, allows for a larger diversity of products, which may have anti-microbial, anti-cancer, anti-mosquito, or anti-obesity activity.

In one embodiment, a method to prepare β-lactones in vitro is provided. The method may employ isolated biosynthetic enzymes (a cell-free method) or host cells expressing one or more heterologous biosynthetic enzymes. In one embodiment, the method includes combining one or more distinct substrates with OleC but not OleD or OleB, or OleA or a homolog thereof, OleC or a homolog thereof and OleD or a homolog thereof but not OleB, so as to yield one or more distinct oxetan-2-ones, wherein the one or more distinct substrates include one or more distinct 3-hydroxy acids, one or more distinct acyl CoAs, one or more distinct carboxylic acids, or one or more distinct fatty acids. As used herein, “distinct” means that there is a difference in the chemical composition of substances. For instance, the method may employ two different acyl CoAs (R1-CoA and R2-CoA where R1 and R2 are distinct acyl groups) which may result in a mixture of oxetan-2-ones, one having two R1s, another having two R2s and yet another having R1 and R2. A mixture of otherwise identical cis and trans isomers of an oxetan-2-one (for example, oxetan-2-ones derived from combining enzymes with R1-CoA) is not distinct oxetan-2-ones. In one embodiment, the one or more distinct 3-hydroxy acids are combined with OleC but not OleD or OleB. In one embodiment, the one or more distinct acyl CoAs are combined with OleA, OleC and OleD but riot OleB. In one embodiment, the one or more distinct acyl CoAs are prepared by combining one or more distinct carboxylic acids, CoA and a ligase. In one embodiment, OleC, or OleA, OleD or OleC or any combination thereof, are expressed in a heterologous cell. In one embodiment, the heterologous cell is a bacterial cell, a fungal cell, or a yeast cell. In one embodiment, OleC or one or more of OleA, OleD or OleC is isolated OleA, OleD or OleC. In one embodiment, the combination of these enzymes yields a plurality of distinct oxetan-2-ones and olefins. In one embodiment, the oxetan-2-one has formula (I):

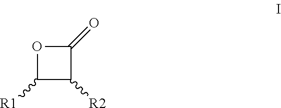

wherein each of R1 and R2 independently is an alkyl, alkenyl, alkynyl, or aryl, which is optionally substituted, e.g., with groups including hydroxyl. In one embodiment, OleA is combined with the one or more distinct acyl CoAs before combining with OleC and OleD so as to increase the relative ratio of trans β-lactones. In one embodiment, at least one of OleA, OleC and OleD is from a different organism. For example, OleA and OleD may be from Xanthomonas and OleC from Stentrophomonas, or OleA may be from Xanthomonas and OleC and OleD from Stentrophomonas, or OleC and OleD may be from Xanthomonas and OleA from Stentrophomonas. In one embodiment, an ATP regenerating system is combined with the enzyme(s) and substrate(s). In one embodiment, OleA, OleC and OleD are combined with fatty acids, CoA and a fatty acyl-CoA synthetase. In one embodiment, OleA, OleC and OleD are combined with fatty acyl-CoAs and isolated lipase, proteosomes, penicillin binding proteins, bacteria, fungi, yeast, or cancer cells, to detect whether the synthesized oxetan-2-one inhibits the lipase, proteosomes, penicillin binding proteins, bacteria, fungi, yeast, or cancer cells.

The biosynthetic enzymes useful in the methods include but are not limited to enzymes that are structurally or functionally related to OleA (encoded by SEQ ID NO:1), OleB (encoded by SEQ ID NO:2), OleC (encoded by SEQ ID NO:3 or having SEQ ID NO:5), and/or OleD (encoded by SEQ ID NO:4), e.g., enzymes having at least 70%, 75%, 80%, 85%, 90%, 92%, 94%, 95%, e.g., 96%, 97%, 98% or 99%, amino acid sequence identity to a polypeptide encoded by one of SEQ ID Nos. 1-4, or SEQ ID NO:5, or a homolog of those polypeptides, e.g., polypeptides having at least 70%, 75%, 80%, 85%, 90%, 92%, 94%, 95%, e.g., 96%, 97%, 98% or 99%, amino acid sequence identity to SEQ ID Nos. 15-21. As used herein, “OleA” includes an enzyme with the activity (an enzyme performing a Claisen condensation of two acyl-CoAs to form a β-keto acid) but not necessarily the specificity of the polypeptide encoded by SEQ NO:1 and having at least 70%, 75%, 80%, 85%, 90%, 92%, 94%, 95%, e.g., 96%, 97%, 98% or 99%, amino acid sequence identity to a polypeptide encoded by SEQ ID NO: l. An exemplary homolog of OleA is LstA (SEQ ID NO:15) including polypeptides at least 70%, 75%, 80%, 85%, 90%, 92%, 94%, 95%, e.g., 96%, 97%, 98% or 99%, amino acid sequence identity to SEQ ID NO:15. LstA and LstB form a heterodimer (LstB is a homolog of OleA not OleB). As used herein, “OleB” includes an enzyme with the activity (β-lactone decarboxylase) but not necessarily the specificity of a polypeptide encoded by SEQ ID NO:2 and having at least 70%, 75%, 80%, 85%, 90%, 92%, 94%, 95%, e.g., 96%, 97%, 98% or 99%, amino acid sequence identity to a polypeptide encoded by SEQ ID NO:2. An exemplary homolog of OleB includes polypeptides at least 70%, 75%, 80%, 85%, 90%, 92%, 94%, 95%, e.g., 96%, 97%, 98% or 99%, amino acid sequence identity to a polypeptide encoded by SEQ ID Nos. 13 or 14. As used herein, “OleC” includes an enzyme with the activity (β-lactone synthetase) but not necessarily the specificity of a polypeptide encoded by SEQ ID NO:3 or having SEQ ID NO:5 and having at least 70%, 75%, 80%, 85%, 90%, 92%, 94%, 95%, e.g., 96%, 97%, 98% or 99%, amino acid sequence identity to a polypeptide encoded by SEQ ID NO:3 or having SEQ ID NO:5. An exemplary homolog of OleC includes polypeptides at least 70%, 75%, 80%, 85%, 90%, 92%, 94%, 95%, e.g., 96%, 97%, 98% or 99%, amino acid sequence identity to one of SEQ ID Nos. 17-21 or encoded by SEQ ID NO: 11 or 12. As used herein, “OleD” includes an enzyme with the activity (catalyzing the NADPH-dependent reduction of a beta keto acid to produce a β-hydroxy acid) but not necessarily the specificity of a polypeptide encoded by SEQ ID NO:4 and having at least 70%, 75%, 80%, 85%, 90%, 92%, 94%, 95%, e.g., 96%, 97%, 98% or 99%, amino acid sequence identity to a polypeptide encoded by SEQ ID NO:4. An exemplary homolog of OleD is LstD (SEQ ID NO:16) including polypeptides at least 70%, 75%, 80%, 85%, 90%, 92%, 94%, 95%, e.g., 96%, 97%, 98% or 99%, amino acid sequence identity to SEQ ID NO:16.

In one embodiment, one or more Ole enzymes are employed to prepare β-lactones using, for example, synthetic substrates. In one embodiment, the disclosure provides a method to produce β-lactones from corresponding 3-hydroxy acid precursors using enzymes in vitro. In one embodiment, the disclosure provides a method for making β-lactones, e.g., lipstatin or ebelactone, with a β-lactone synthetase, e.g., OleC, from 3-hydroxy acid precursors. In one embodiment, the disclosure provides a method for making β-lactones with OleC, OleA and OleD from acyl-CoA precursors. In one embodiment, the disclosure provides a method for making β-lactones with OleC, OleA, OleD and fatty acyl-CoA synthetase from fatty acid precursors. In one embodiment, the disclosure provides a method for making β-lactones as described above but allowing racemization of the OleA product to occur so as to increase the preponderance of trans-β-lactones, in one embodiment, the disclosure provides a method for using LstD or NltD to produce trans-β-lactones.

In one embodiment, the disclosure provides the use of an OleABCD system, in vitro or in vivo, in which OleB (a β-lactone decarboxylase that destroys β-lactones) is mutated, e.g., by site-directed methods in vitro or in vivo using for instance CRISPR-Cas9 or TALEN technology, such that OleB activity is blocked, or OleB is otherwise blocked in vivo, and β-lactones accumulate.

In one embodiment, the disclosure provides a combinatorial method using mixtures of enzymes from different sources to make large numbers of β-lactones in one reaction vessel for large scale combinatorial screening.

In one embodiment, the disclosure provides a method for scaling up enzymatic production such that desirable β-lactones can be made in vitro in microgram, milligram, gram, and kilogram quantities.

Further provided are host cells that recombinantly express one of more Ole enzymes, and uses thereof.

In one embodiment, the disclosure provides kits having at least two distinct substrates, one or more Ole enzymes, or at least one substrate and at least one Ole enzyme.

In one embodiment, the disclosure provides an assay that can be used to identify β-lactone synthetases in vitro and in vivo. The assay may be employed for screening and in a high-throughput manner. The method includes combining at room temperature, e.g., from about 19° C. to about 27° C., and a pH of about 6 to about 8, a sample suspected of having β-lactone synthetase and a dialkene or dialkyne so as to yield a mixture; and detecting in the mixture a change in UV absorbance over time, wherein a change in UV absorbance is indicative of the presence or amount of a β-lactone synthetase. In one embodiment, the assay employs a β-lactone synthetase substrate with two C═C bonds conjugated with the produced β-lactone or subsequent alkene (see FIG. 6). The β-lactone is unstable and so spontaneously decarboxylates at room temperature and pH 7, thus forming a triene with a very high extinction coefficient that can readily be detected spectrophotametrically in a cuvette or in a micro-titer well plate. Another comparable substrate with two conjugated triple bonds reacts similarly.

### BRIEF DESCRIPTION OF FIGURES

FIG. 1. Ole enzymes make β-lactones.

FIG. 2. Homologous OleC enzymes encoded in β-lactone biosynthesis gene clusters. Percent identity is based on amino acid sequences. The E-values for OleC to LstC and Orf1 are 2×10⁻⁷² and 1×10⁻¹⁴³, respectively. The bit scores for OleC to LstC and Orf1 are 340 and 435, respectively. Lipstatin is the precursor to the anti-obesity drug Orlistat. Ebelactone A is a commercially available general esterase inhibitor.

FIG. 3A. Generic structures and precursors.

FIG. 3B. Enzyme strategies that employ different precursors (substrates) and OleC, OleB, or a combination of OleA, OleD and OleC, and optionally a ligase.

FIG. 4. Exemplary substrates and products produced by OleC.

FIG. 5. Exemplary alkane substrates for OleC in C8-C10 range.

FIG. 6. Exemplary assay to detect OleC activity.

FIGS. 7A-C. OleB decarboxylation of cis-β-lactones to cis-olefins followed by ¹H nuclear magnetic resonance spectroscopy (NMR). (A) ¹H-NMR showing synthetic standards of cis- and trans-3-octyl-4-nonyloxetan-2-one and cis-9-nonadecene. B) ¹H-NMR for reaction of OleB with synthetic trans-β-lactone minor cis-β-lactone centered at 4.55 shown) showing no reaction towards this enantiomeric pair. The small peak for cis-olefin is believed to originate from the minor cis-β-lactone. contaminant. C) OleB+cis-β-lactone showing approximately half of the starting material has been converted to cis-olefin suggesting that only one of the cis-enantiomers reacted with OleB.

FIGS. 8A-C. Sequence analysis and structural modeling of OleB and haloalkane dehalogenase proteins. A) Phylogenetic tree of OleB and OIeBC sequences aligned with characterized members of the HLD family separated into classes I, II and III (Chovancova et al., 2007). OleB and OleBC sequences cluster with the HLD class III Rhodopirellula baltica sequence. There were a total of 13 sequences included in the final alignment. Unrooted maximum-likelihood tree was estimated using the Jones Taylor Thornton model of amino acid evolution. Bootstrap values are displayed at each node (100 data resamplings). Scale bar represents 0.1 changes per amino-acid position. B) Multiple sequence alignment revealed a putative catalytic triad of amino acids. Nunibering of the proposed catalytic triad at the top is based on the amino acid position in the Xanthomonas campestris OleB sequence (WP_012437021.1) studied here. C) Specific structures for the HLD I and HLD II.

FIG. 9. OleB forms a stable acyl-enzyme intermediate when reacted with 7-(bromomethyl)pentadecane. OleB show a mass shift about 222 m/z consistent the nucleophilic attack and displacement of bromine with the substrate. The mass shift expected with the loss of bromide is 225 m/z. OleB_{D114A} did not show any mass shift when reacted with the bromo-alkane substrate. This data is consistent with a haloalkane dehalogenase like mechanism. The two major peaks and one minor peak appear in all OleB and OleB mutant MALDI-TOF experiments and are presumably the result of an ion with a m/z of 180.

FIGS. 10A-B. Natural OleBC fusion from Micrococcus luteus when reacted with β-hydroxy acids. A) Wild-type OleBC fusion accumulates trans-β-lactone as well as equivalent amounts of cis-olefin and cis-β-lactone constant with only one of the cis-β-lactone enantiomers reacted with by the OleB domain. B) The functional OleC domain of OleB_{D114A}C can convert syn- and anti-β-hydroxy acids to cis- and trans-β-lactones respectively, but the mutant OleB domain does not generate cis-olefin.

FIG. 11. Alignment of OleB and OleBC fusion proteins within bacterial α/β-hydrolase enzymes. FIG. 12. OleB proteins are encoded in oleABCD gene clusters; however, many were annotated as haloalkane dehalogenases in subfamily III (HLD-III). The OleB domain of OleBC fusion proteins like Micrococcus luteus were not included in the alignments, but clusters within the HLD III subgroup were included.

FIGS. 13A-E. A) Orlistat inhibition of lipase. B) Cis-lactone inhibition of lipase. C) Trans-lactone inhibition of lipase. D) Inhibitor comparison. E) Lipase inhibition by p-lactone products of 96-well reactions with OleC and β-hydroxy acid precursors.

FIG. 14. OleC-like homologs are widely distributed in the tree of life.

FIG. 15. Pipeline for bioinformatic analysis of oleABCD gene clusters.

FIG. 16. Exemplary homologs of OleC (SEQ ID Nos.17-21). See also SEQ ID NO:25.

FIG. 17. A) Synteny between olefin, nocardiolactone, and lipstatin biosynthetic gene clusters. B) Olefin and nocardiolactone pathways are similar but differ in β-lactone stereochemistry and the presence of a β-lactone decarboxylase, OleB. R₁50 C₉H₁₉, R2═C₈H₁₇.

FIG. 18. OleD and MD can be swapped in one-pot enzyme reactions to control stereochemistry and produce exclusively cis- (black) and/or trans-β-lactones (gray), respectively. An equimolar mixture of the two enzymes yields a mixture of cis- and trans-β-lactone products, with higher production of cis- likely due to higher enzyme efficiency. Data are represented as average peak area±SEM. R₁═C₉H₁₉, R₂═C₈H₁₇.

FIG. 19. Summary of reactions catalyzed.

FIG. 20. OleA and esters used to make libraries.

FIG. 21. Exemplary substrate to prepare a β-lactone (formula (I)) and anexemplary substrate to detect beta-lactone synthetases (formula (II)).

FIG. 22. Two exemplary assays to detect β-lactone synthetase.

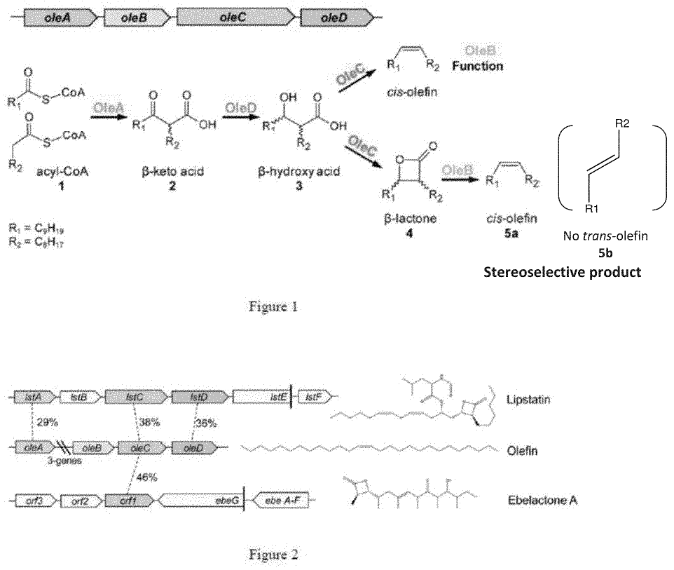

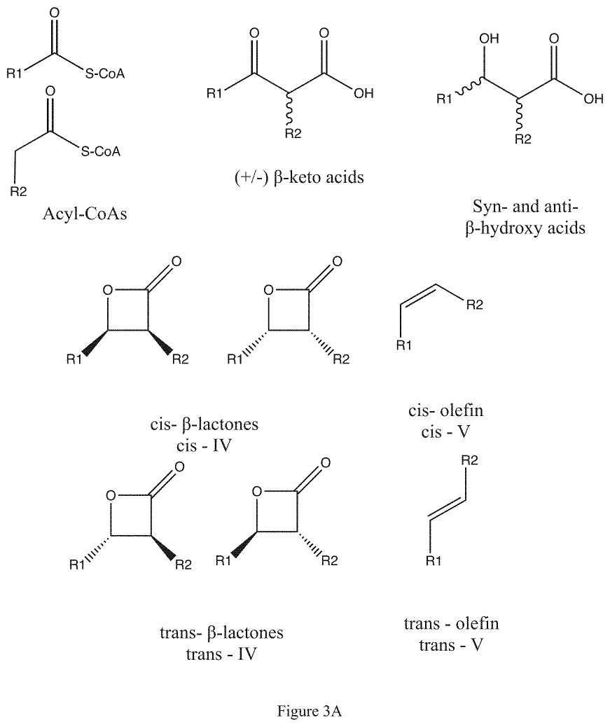

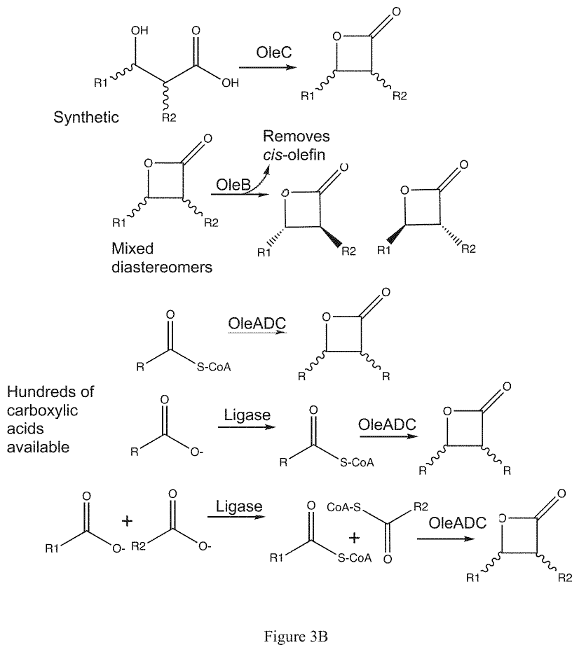

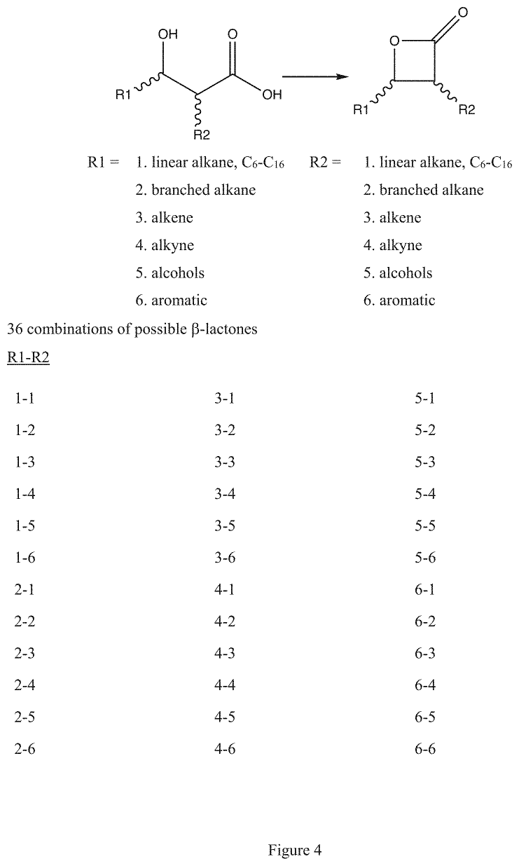

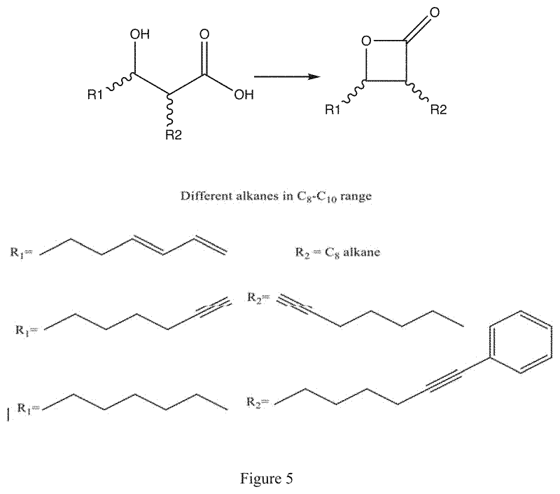

The patent or application file contains at least one drawing executed in color. Copies of this patent or patent application publication with color drawing(s) will be provided by the Office upon request and payment of the necessary fee.

### DETAILED DESCRIPTION

### Definitions

An “expression vector” is a vector comprising a region which encodes a polypeptide of interest, and is used for effecting the expression of the protein in an intended target cell. An expression vector also comprises control elements operatively linked to the encoding region to facilitate expression of the protein in the target. The combination of control elements and a gene or genes to which they are operably linked for expression is sometimes referred to as an “expression cassette,” a large number of which are known and available in the art or can be readily constructed from components that are available in the art. “Gene delivery,” “gene transfer,” and the like as used herein, are terms referring to the introduction of an exogenous polynucleotide (sometimes referred to as a “transgene”) into a host cell, irrespective of the method used for the introduction. Such methods include a variety of well-known techniques such as vector-mediated gene transfer (by, e.g., viral infection/transfection, or various other protein-based or lipid-based gene delivery complexes) as well as techniques facilitating the delivery of “naked” polynucleotides (such as electroporation, “gene gun” delivery and various other techniques used for the introduction of polynucleotides). The introduced polynucleotide may be stably or transiently maintained in the host cell. Stable maintenance typically requires that the introduced polynucleotide either contains an origin of replication compatible with the host cell or integrates into a replicon of the host cell such as an extrachromosomal replicon (e.g., a plasmid) or a nuclear or mitochondrial chromosome. A number of vectors are known to be capable of mediating transfer of genes to mammalian cells, as is known in the art.

“Heterologous” means derived from a genotypically distinct entity from that of the rest of the entity to which it is compared. For example, a polynucleotide introduced by genetic engineering techniques into a different cell type is a heterologous polynucleotide (and, when expressed, can encode a heterologous polypeptide). Similarly, a TRS or promoter that is removed from its native coding sequence and operably linked to a different coding sequence is a heterologous TRS or promoter.

The term “heterologous” as it relates to nucleic acid sequences such as gene sequences and control sequences, denotes sequences that are not normally joined together, and/or are not normally associated with a particular cell. Thus, a “heterologous” region of a nucleic acid construct or a vector is a segment of nucleic acid within or attached to another nucleic acid molecule that is not found in association with the other molecule in nature. For example, a heterologous region of a nucleic acid construct could include a coding sequence flanked by sequences not found in association with the coding sequence in nature, i.e., a heterologous promoter. Another example of a heterologous coding sequence is a construct where the coding sequence itself is not found in nature (e.g., synthetic sequences having codons different from the native gene). Similarly, a cell transformed with a construct which is not normally present in the cell would be considered heterologous for purposes of this invention.

The term “exogenous,” when used in relation to a protein, gene, nucleic acid, or polynucleotide in a cell or organism refers to a protein, gene, nucleic acid, or polynucleotide which has been introduced into the cell or organism by artificial or natural means, or in relation a cell refers to a cell which was isolated and subsequently introduced to other cells or to an organism by artificial or natural means. An exogenous nucleic acid may be from a different organism or cell, or it may be one or more additional copies of a nucleic acid which occurs naturally within the organism or cell. An exogenous cell may be from a different organism, or it may be from the same organism. By way of a non-limiting example, an exogenous nucleic acid is in a chromosomal location different from that of natural cells, or is otherwise flanked by a different nucleic acid sequence than that found in nature.

The term“isolated” when used in relation nucleic acid, peptide, or polypeptide refers to a nucleic acid sequence, peptide, or polypeptide that is identified and separated from at least one contaminant nucleic acid, polypeptide or other biological component with which it is ordinarily associated in its natural source. Isolated nucleic acid, peptide, or polypeptide is present in a form or setting that is different from that in which it is found in nature. For example, a given DNA sequence (e.g., a gene) is found on the host cell chromosome in proximity to neighboring genes; RNA sequences, such as a specific snRNA sequence encoding a specific protein, are found in the cell as a mixture with numerous other mRNAs that encode a multitude of proteins. The isolated nucleic acid molecule may be present in single-stranded or double-stranded form. When an isolated nucleic acid molecule is to be utilized to express a protein, the molecule will contain at a minimum the sense or coding strand (i.e., the molecule may single-stranded), but may contain both the sense and anti-sense strands (i.e., the molecule may be double-stranded). For example, an isolated substance may be prepared by using a purification technique to enrich it from a source mixture. Enrichment can be measured on an absolute basis, such as weight per volume of solution, or it can be measured in relation to a second, potentially interfering substance present in the source mixture.

As used herein, “substantially pure” means an object species is the predominant species present (i.e., on a molar basis it is more abundant than any other individual species in the composition), and preferably a substantially purified fraction is a composition wherein the object species comprises at least about 50 percent (on a molar basis) of all macromolecular species present. Generally, a substantially pure composition will comprise more than about 80 percent of all macromolecular species present in the composition, more preferably more than about 85%, about 90%, about 95%, and about 99%. Most preferably, the object species is purified to essential homogeneity (contaminant species cannot be detected in the composition h conventional detection methods) wherein the composition consists essentially of a single macromolecular species.

The term “polynucleotide” refers to a polymeric form of nucleotides of any length, including deoxyribonucleotides or ribonucleotides, or analogs thereof. A polynucleotide may comprise modified nucleotides, such as methylated or capped nucleotides and nucleotide analogs, and may be interrupted by non-nucleotide components. If present, modifications to the nucleotide structure may be imparted before or after assembly of the polymer. The term polynucleotide, as used herein, refers interchangeably to double- and single-stranded molecules. Unless otherwise specified or required, any embodiment of the invention described herein that is a polynucleotide encompasses both the double-stranded form and each of two complementary single-stranded forms known or predicted to make up the double-stranded form.

In general, “substituted” refers to an organic group as defined herein in which one or more bonds to a hydrogen atom contained therein are replaced by one or more bonds to a non-hydrogen atom such as, but not limited to, a halogen F, Cl, Br, and I); an oxygen atom in groups such as hydroxyl groups, alkoxy groups, aryloxy groups, aralkyloxy groups, oxo(carbonyl) groups, carboxyl groups including carboxylic acids, carboxylates, and carboxylate esters; a sulfur atom in groups such as thiol groups, alkyl and aryl sulfide groups, sulfoxide groups, sulfone groups, sulfonyl groups, and sulfonamide groups; a nitrogen atom in groups such as amines, hydroxylamines, nitriles, nitro groups, N-oxides, hydrazides, azides, and enamines; and other heteroatoms in various other groups. Non-limiting examples of substituents that can be bonded to a substituted carbon (or other) atom include F, Cl, Br, I, OR′, OC(O)N(R′)₂, CN, NO, NO₂, ONO₂, azido, CF₃, OCF₃, R′, O (oxo), S (thiono), methylenedioxy, ethylenedioxy, N(R′)₂, SR′, SOR′, SO₂R′, SO₂N(R′)₂, SO₃R′, C(O)R′, C(O)C(O)R′, C(O)CH₂C(O)R′, C(S)R′, C(O)OR′, OC(O)R′, C(O)N(R′)₂, OC(O)N(R′)₂, C(S)N(R′)₂, (CH₂)₀₋₂N(R′)C(O)R′, (CH₂)₀₋₂N(R′)N(R′)₂, N(R′)N(R′)C(O)R′, N(R)N(R)C(O)OR′, N(R′)N(R′)CON(R′)₂, N(R′)SO₂R′, N(R′)SO₂N(R′)₂, N(R′)C(O)OR′, N(R′)C(O)R′, N(R′)C(S)R′, N(R′)C(O)N(R)₂, N(R′)C(S)N(R′)₂, N(COR′)COR′, N(OR′)R′, C(═NH)N(R′)₂, C(O)N(OR′)R′, or C(═NOR′)R′ wherein R′ can be hydrogen or a carbon-based moiety, and wherein the carbon-based moiety can itself be further substituted.

When a substituent is monovalent, such as, for example, F or Cl, it is bonded to the atom it is substituting by a single bond. When a substituent is more than monovalent, such as O, which is divalent, it can be bonded to the atom it is substituting by more than one bond, i.e., a divalent substituent is bonded by a double bond; for example, a C substituted with 0 forms a carbonyl group, C═O, which can also be written as “CO”, “C(O)”, or “C(═O)”, wherein the C and the O are double bonded. When a carbon atom is substituted with a double-bonded oxygen (═O) group, the oxygen substituent is termed an “oxo” group. When a divalent substituent such as NR is double-bonded to a carbon atom, the resulting C(═NR) group is termed an “imino” group. When a divalent substituent such as S is double-bonded to a carbon atom, the results C(═S) group is termed a “thiocarbonyl” group.

Alternatively, a divalent substituent such as O or S can be connected by two single bonds to two different carbon atoms. For example, O, a divalent substituent, can be bonded to each of two adjacent carbon atoms to provide an epoxide group, or the O can form a bridging ether group, termed an “oxy” group, between adjacent or non-adjacent carbon atoms, for example bridging the 1,4-carbons of a cyclohexyl group to form a [2.2.1]-oxabicyclo system. Further, any substituent can be bonded to a carbon or other atom by a linker, such as (CH₂), or (CR′₂)ₙ wherein n is 1, 2, 3, or more, and each R′ is independently selected. Similarly, a methylenedioxy group can be a substituent when bonded to two adjacent carbon atoms, such as in a phenyl ring.

C(O) and S(O)₂ groups can be bound to one or two heteroatoms, such as nitrogen, rather than to a carbon atom. For example, when a C(O) group is bound to one carbon and one nitrogen atom, the resulting group is called an “amide” or “carboxamide.” When a C(O) group is bound to two nitrogen atoms, the functional group is termed a urea. When a S(O)₂ group is bound to one carbon and one nitrogen atom, the resulting unit is termed a “sulfonamide.” When a S(O)₂ group is bound to two nitrogen atoms, the resulting unit is termed a “sulfamate.”

Substituted alkyl, alkenyl, alkynyl, cycloalkyl, and cycloalkenyl groups as well as other substituted groups also include groups in which one or more bonds to a hydrogen atom are replaced by one or more bonds, including double or triple bonds, to a carbon atom, or to a heteroatom such as, but not limited to, oxygen in carbonyl (oxo), carboxyl, ester, amide, halide., urethane, and urea groups; and nitrogen in imines, hydroxyimines, oximes, hydrazones, amidines, guanidines, and nitriles.

Substituted ring groups such as substituted cycloalkyl, aryl, heterocyclyl and heteroaryl groups also include rings and fused ring systems in which a bond to a hydrogen atom is replaced with a bond to a carbon atom. Therefore, substituted cycloalkyl, aryl, heterocyclyl and heteroaryl groups can also be substituted with alkyl alkenyl, and alkynyl groups as defined herein.

By a “ring system” as the term is used herein is meant a moiety comprising one, two, three or more rings, which can be substituted with non-ring groups or with other ring systems, or both, which can be fully saturated, partially unsaturated, fully unsaturated, or aromatic, and when the ring system includes more than a single ring, the rings can be fused, bridging, or spirocyclic. By “spirocyclic” is meant the class of structures wherein two rings are fused at a single tetrahedral carbon atom, as is well known in the art.

As to any of the groups described herein, which contain one or more substituents, it is understood, of course, that such groups do not contain any substitution or substitution patterns which are sterically impractical and/or synthetically non-feasible. In addition, the compounds of this disclosed subject matter include all stereochemical isomers arising from the substitution of these compounds.

Alkyl groups include straight chain and branched alkyl groups and cycloalkyl groups having from 1 to about 20 carbon atoms, and typically from 1 to 12 carbons or, in some embodiments, from 1 to 8 carbon atoms. Examples of straight chain alkyl groups include those with from 1 to 8 carbon atoms such as methyl, ethyl, n-propyl, n-butyl, n-pentyl, n-hexyl, n-heptyl, and n-octyl groups. Examples of branched alkyl groups include, but are not limited to, isopropyl, iso-butyl, sec-butyl, t-butyl, neopentyl, isopentyl, and 2,2-dimethylpropyl groups. Representative substituted alkyl groups can be substituted one or more times with any of the groups listed above, for example, amino, hydroxy, cyano, carboxy, nitro, thio, alkoxy, and halogen groups.

Cycloalkyl groups are cyclic alkyl groups such as, but not limited to, cyclopropyl, cyclobutyl, cyclopentyl, cyclohexyl, cycloheptyl, and cyclooctyl groups. In some embodiments, the cycloalkyl group can have 3 to about 8-12 ring members, whereas in other embodiments the number of ring carbon atoms range from 3 to 4, 5, 6, or 7. Cycloalkyl groups further include polycyclic cycloalkyl groups such as, but not limited to, norbornyl, adamantyl, bornyl, camphenyl, isocamphenyl, and carenyl groups, and fused rings such as, but not limited to, decalinyl, and the like. Cycloalkyl groups also include rings that are substituted with straight or branched chain alkyl groups as defined above. Representative substituted cycloalkyl groups can be mono-substituted or substituted more than once, such as, but not limited to, 2,2-, 2,3-, 2,4-2,5- or 2,6-disubstituted cyclohexyl groups or mono-, di- or tri-substituted norbornyl or cycloheptyl groups, which can be substituted with, for example, amino, hydroxy, cyano, carboxy, nitro, thio, alkoxy, and halogen groups. The term “cycloalkenyl” alone or in combination denotes a cyclic alkenyl group.

The terms “carbocyclic,” “carbocyclyl,” and “carbocycle” denote a ring structure wherein the atoms of the ring are carbon, such as a cycloalkyl group or an aryl group. In sonic embodiments, the carbocycle has 3 to 8 ring members, whereas in other embodiments the number of ring carbon atoms is 4, 5, 6, or 7. Unless specifically indicated to the contrary, the carbocyclic ring can be substituted with as many as N-1 substituents wherein N is the size of the carbocyclic ring with, for example, alkyl, alkenyl, alkynyl, amino, aryl, hydroxy, cyano, carboxy, heteroaryl, heterocyclyl, nitro, thio, alkoxy, and halogen groups, or other groups as are listed above. A carbocyclyl ring can be a cycloalkyl ring, a cycloalkenyl ring, or an aryl ring. A carbocyclyl can be monocyclic or polycyclic, and if polycyclic each ring can be independently be a cycloalkyl ring, a cycloalkenyl ring, or an aryl ring.

(Cycloalkyl)alkyl groups, also denoted cycloalkylalkyl, are alkyl groups as defined above in which a hydrogen or carbon bond of the alkyl group is replaced with a bond to a cycloalkyl group as defined above.

Alkenyl groups include straight and branched chain and cyclic alkyl groups as defined above, except that at least one double bond exists between two carbon atoms. Thus, alkenyl groups have from 2 to about 20 carbon atoms, and typically from 2 to 12 carbons or, in some embodiments, from 2 to 8 carbon atoms. Examples include, but are not limited to vinyl, —CH═CH(CH₃), —CH═C(CH₃)₂, —C(CH₃)═CH₂, —C(CH₃)═CH(CH₃), —C(CH₂CH₃)═CH₂, cyclohexenyl, cyclopentenyl, cyclohexadienyl, butadienyl, pentadienyl, and hexadienyl among others.

Cycloalkenyl groups include cycloalkyl groups having at least one double bond between 2 carbons. Thus for example, cycloalkenyl groups include but are not limited to cyclohexenyl, cyclopentenyl, and cyclohexadienyl groups. Cycloalkenyl groups can have from 3 to about 8-12 ring members, whereas in other embodiments the number of ring carbon atoms range from 3 to 5, 6, or 7. Cycloalkyl groups further include polycyclic cycloalkyl groups such as, but not limited to, norbornyl, adamantyl, bornyl, camphenyl, isocamphenyl, and carenyl groups, and fused rings such as, but not limited to, decalinyl, and the like, provided they include at least one double bond within a ring. Cycloalkenyl groups also include rings that are substituted with straight or branched chain alkyl groups as defined above.

(Cycloalkenyl)alkyl groups are alkyl groups as defined above in which a hydrogen or carbon bond of the alkyl group is replaced with a bond to a cycloalkenyl group as defined above.

Alkynyl groups include straight and branched chain alkyl groups, except that at least one triple bond exists between two carbon atoms. Thus, alkynyl groups have from 2 to about 20 carbon atoms, and typically from 2 to 12 carbons or, in some embodiments, from 2 to 8 carbon atoms. Examples include, but are not limited to —CH≡CH, —C≡C(CH₃), —C≡C(CH₂CH₃), —CH₂C≡CH, —CH₂C═C(CH₃), and —CH₂C≡C(CH₂CH₃) among others.

The term “heteroalkyl” by itself or in combination with another term means, unless otherwise stated, a stable straight or branched chain alkyl group consisting of the stated number of carbon atoms and one or two heteroatoms selected from the group consisting of O, N, and S, and wherein the nitrogen and sulfur atoms may be optionally oxidized and the nitrogen heteroatom may be optionally quaternized. The heteroatom(s) may be placed at any position of the heteroalkyl group, including between the rest of the heteroalkyl group and the fragment to which it is attached, as well as attached to the most distal carbon atom in the heteroalkyl group. Examples include: —O—CH₂—CH₂—CH₃, —CH₂—CH₂CH₂—OH, —CH₂—CH₂—NH—CH₃, —CH₂—S—CH₂—CH₃, —CH₂S(═O)—CH₃, and —CH₂CH₂O—CH₂CH₂—O—CH₃. Up to two heteroatoms may be consecutive, such as, for example, —CH₂—NH—OCH₃, or —CH₂—CH₂—S—S—CH₃.

A “cycloheteroalkyl” ring is a cycloalkyl ring containing at least one heteroatom. A cycloheteroalkyl ring can also be termed a “heterocyclyl,” described below.

The term “heteroalkenyl” by itself or in combination with another term means, unless otherwise stated, a stable straight or branched chain monounsaturated or di-unsaturated hydrocarbon group consisting of the stated number of carbon atoms and one or two heteroatoms selected from the group consisting of O, N, and S, and wherein the nitrogen and sulfur atoms may optionally be oxidized and the nitrogen heteroatom may optionally be quaternized. Up to two heteroatoms may be placed consecutively. Examples include —CH═CH—O—CH₃, —CH═CH—CH₂—OH, —CH₂—CH═N—OCH₃, —CH═CH—N(CH₃)—CH₃, —CH₂—CH═CH—CH₂—SH , and —CH═CH—O—CH₂CH₂—O—CH₃.

Aryl groups are cyclic aromatic hydrocarbons that do not contain heteroatoms in the ring. Thus aryl groups include, but are not limited to, phenyl, azulenyl, heptalenyl, biphenyl, indacenyl, fluorenyl, phenanthrenyl, triphenylenyl, pyrenyl, naphthacenyl, chrysenyl, biphenylenyl, anthracenyl, and naphthyl groups. In some embodiments, aryl groups contain about 6 to about 14 carbons in the ring portions of the groups. Aryl groups can be unsubstituted or substituted, as defined above. Representative substituted aryl groups can be mono-substituted or substituted more than once, such as, but not limited to, 2-, 3-, 4-, 5-, or 6-substituted phenyl or 2-8 substituted naphthyl groups, which can be substituted with carbon or non-carbon groups such as those listed above.

Aralkyl groups are alkyl groups as defined above in which a hydrogen or carbon bond of an alkyl group is replaced with a bond to an aryl group as defined above. Representative aralkyl groups include benzyl and phenylethyl groups and fused (cycloalkylaryl)alkyl groups such as 4-ethyl-indanyl. Aralkenyl groups are alkenyl groups as defined above in which a hydrogen or carbon bond of an alkyl group is replaced with a bond to an aryl group as defined above. Aralkynyl groups are alkynl groups as defined above in which a hydrogen or carbon bond of an alkynl group is replaced with a bond to an aryl group as defined above.

Heterocyclyl groups or the term “heterocyclyl” includes aromatic and non-aromatic ring compounds containing 3 or more ring members, of which, one or more is a heteroatom such as, but not limited to, N, O, and S. Thus a heterocyclyl can be a cycloheteroalkyl, or a heteroaryl, or if polycyclic, any combination thereof. In some embodiments, heterocyclyl groups include 3 to about 20 ring members, whereas other such groups have 3 to about 15 ring members. A heterocyclyl group designated as a C₂-heterocyclyl can be a 5-ring with two carbon atoms and three heteroatoms, a 6-ring with two carbon atoms and four heteroatoms and so forth. Likewise a C₄-heterocyclyl can be a 5-ring with one heteroatom, a 6-ring with two heteroatoms, and so forth. The number of carbon atoms plus the number of heteroatoms sums up to equal the total number of ring atoms. A heterocyclyl ring can also include one or more double bonds. A heteroaryl ring is an embodiment of a heterocyclyl group. The phrase “heterocyclyl group” includes fused ring species including those comprising fused aromatic and non-aromatic groups. For example, a dioxolanyl ring and a benzdioxolanyl ring system (methylenedioxyphenyl ring system) are both heterocyclyl groups within the meaning herein. The phrase also includes polycyclic ring systems containing a heteroatom such as, but not limited to, quinuclidyl. Heterocyclyl groups can be unsubstituted, or can be substituted as discussed above. Heterocyclyl groups include, but are not limited to, pyrrolidinyl, piperidinyl, piperazinyl, morpholinyl, pyrrolyl, pyrazolyl, triazolyl, tetrazolyl, oxazolyl, isoxazolyl, thiazolyl, pyridinyl, thiophenyl, benzothiophenyl, benzofuranyl, dihydrobenzofuranyl, indolyl, dihydroindolyl, azaindolyl, indazolyl, benzimidazolyl, azabenzimidazolyl, benzoxazolyl, benzothiazolyl, benzothiadiazolyl, imidazopyridinyl, isoxazolopyridinyl, thianaphthalenyl, purinyl, xanthinyl, adeninyl, guaninyl, quinolinyl, tetrahydroquinolinyl, quinoxalinyl, and quinazolinyl groups. Representative substituted heterocyclyl groups can be mono-substituted or substituted more than once, such as, but not limited to, piperidinyl or quinolinyl groups, which are 2-, 3-, 4-, 5-, or 6-substituted, or disubstituted with groups such as those listed above. Heteroaryl groups are aromatic ring compounds containing 5 or more ring members, of which, one or more is a heteroatom such as, but not limited to, N, O, and S; for if instance, heteroaryl rings can have 5 to about 8-12 ring members. A heteroaryl group is a variety of a heterocyclyl group that possesses an aromatic electronic structure. A heteroaryl group designated as a C₂-heteroaryl can be a 5-ring with two carbon atoms and three heteroatoms, a 6-ring with two carbon atoms and four heteroatoms and so forth. Likewise, a C₄-heteroaryl can be a 5-ring with one heteroatom, a 6-ring with two heteroatoms, and so forth. The number of carbon atoms plus the number of heteroatoms sums up to equal the total number of ring atoms. Heteroaryl groups include, but are not limited to, groups such as pyrrolyl, pyrazolyl, triazolyl, tetrazolyl, oxazolyl, isoxazolyl, thiazolyl, pyridinyl, thiophenyl, benzothiophenyl, benzofuranyl, indolyl, azaindolyl, indazolyl, benzimidazolyl, azabenzimidazolyl, benzoxazolyl, benzothiazolyl, benzothiadiazolyl, imidazopyridinyl, isoxazolopyridinyl, thianaphthalenyl, purinyl, xanthinyl, adeninyl, guaninyl, quinolinyl, isoquinolinyl, tetrahydroquinolinyl, quinoxalinyl, and quinazolinyl groups. Heteroaryl groups can be unsubstituted, or can be substituted with groups as is discussed above. Representative substituted heteroaryl groups can be substituted one or more times with groups such as those listed above.

Additional examples of aryl and heteroaryl groups include but are not limited to phenyl, biphenyl, indenyl, naphthyl (1-naphthyl, 2-naphthyl), N-hydroxytetrazolyl, N-hydroxytriazolyl, N-hydroxyimidazolyl, anthracenyl anthracenyl, 2-anthracenyl, 3-anthracenyl), thiophenyl (2-thienyl, 3-thienyl), furyl (2-furyl, 3-furyl), indolyl, oxadiazolyl, isoxazolyl, quinazolinyl, thorenyl, xanthenyl, isoindanyl, benzhydryl, acridinyl, thiazolyl, pyrrolyl (2-pyrrolyl), pyrazolyl (3-pyrazolyl), imidazolyl (1-imidazolyl , 2-imidazolyl, 4-imidazolyl, 5-imidazolyl), triazolyl(1,2,3-triazol-1-yl, 1,2,3-triazol-2-yl 1,2,3-triazol-4-yl, 1,2,4-triazol-3-yl), oxazolyl(2-oxazolyl, 4-oxazolyl, 5-oxazolyl), thiazolyl (2-thiazolyl, 4-thiazolyl, 5-thiazolyl), pyridyl (2-pyridyl, 3-pyridyl, 4-pyridyl), pyrimidinyl (2-pyrimidinyl, 4-pyrimidinyl, 5-pyrimidinyl, 6-pyrimidinyl), pyrazinyl, pyridazinyl (3-pyridazinyl, 4-pyridazinyl, 5-pyridazinyl), quinolyl(2-quinolyl, 3-quinolyl, 4-quinolyl, 5-quinolyl, 6-quinolyl, 7-quinolyl, 8-quinolyl), isoquinolyl (1-isoquinolyl, 3-isoquinolyl, 6-isoquinolyl, 7-isoquinolyl, 8-isoquinolyl), benzo[b]furanyl (2-benzo[b]furanyl, 3-benzo[b]furanyl, 4-benzo[b]furanyl, 5-benzo[b]furanyl, 6-benzo[b]furanyl, 7-benzo[b]furanyl), 2,3-dihydro-benzo[b]furanyl (2-(2,3-dihydro-benzo[b]furanyl), 3-(2,3-dihydro-benzo[b]furanyl), 4-(2,3-dihydro-benzo[b]furanyl), 5-(2,3-dihydro-benzo[b]furanyl), 6-(2,3-dihydro-benzo[b]furanyl), 7-(2,3-dihydro-benzo[b]furanyl), benzo[b]thiophenyl (2-benzo[b]thiophenyl, 3-benzo[b]thiophenyl, 4-benzo[b]thiophenyl, 5-benzo[b]thiophenyl, 6-benzo[b]thiophenyl, 7-benzo[b]thiophenyl), 2,3-dihydro-benzo[b]thiophenyl, (2-(2,3-dihydro-benzo[b]thiophenyl), 3-(2,3-dihydro-benzo[b]thiophenyl), 4-(2,3-dihydro-benzo[b]thiophenyl), 5-(2,3-dihydro-benzo[b]thiophenyl), 6-(2,3-dihydro-benzo[b]thiophenyl), 7-(2,3-dihydro-benzo[]thiophenyl), indolyl (1-indolyl, 2-indolyl, 3-indolyl, 4-indolyl, 5-indolyl, 6-indolyl, 7-indolyl), indazole (1-indazolyl, 3 indazolyl, 4-indazolyl, 5-indazolyl, 6 indazolyl, 7-indazolyl), benzimidazolyl(1-benzimidazolyl, 2-benzimidazolyl, 4-benzimidazolyl, 5-benzimidazolyl, 6-benzimidazolyl, 7-benzimidazolyl, 8-benzimidazolyl), benzoxazolyl (1-benzoxazolyl, 2-benzoxazolyl), benzothiazolyl (1-benzothiazolyl, 2-benzothiazolyl, 4-benzothiazolyl, 5-benzothiazolyl, 6-benzothiazolyl, 7-benzothiazolyl), carbazolyl (1-carbazolyl, 2-carbazolyl, 3-carbazolyl, 4-carbazolyl), 5H-dibenz[b,f]azepine (5H-dibenz[b,f]azepin-1-yl, 5H-dibenz[b,f]azepine-2-yl, 5H-dibenz[b,f]azepine-3-yl, 5H-dibenz[b,f]azepine-4-yl, 5H-dibenz[b,f]azepine-5-yl), 10,11-dihydro-5H-dibenz[b,f]azepine (10,11-dihydro-5H-dibenz[b,f]azepine-1-yl, 10,11-dihydro-5H-1-dibenz[b,f]azepine-2-yl, 10,11-dihydro-5H-dibenz[b,f]azepine-3-yl, 10,11-dihydro-5H-dibenz[b,f]azepine-4-yl, 10,11-dihydro-5H-dibenz[b,f]azepine-5-yl), and the like.

Heterocyclylalkyl groups are alkyl groups as defined above in which a hydrogen or carbon bond of an alkyl group as defined above is replaced with a bond to a heterocyclyl group as defined above. Representative heterocyclyl alkyl groups include, but are not limited to, furan-2-yl methyl, furan-3-yl methyl, pyridine-3-yl methyl, tetrahydrofuran-2-yl ethyl, and indo1-2-ylpropyl.

Heteroarylalkyl groups are alkyl groups as defined above in which a hydrogen or carbon bond of an alkyl group is replaced with a bond to a heteroaryl group as defined above.

The term “alkoxy” refers to an oxygen atom connected to an alkyl group, including a cycloalkyl group, as are defined above. Examples of linear alkoxy groups include but are not limited to methoxy, ethoxy, propoxy, butoxy, pentyloxy, hexyloxy, and the like. Examples of branched alkoxy include but are not limited to isopropoxy, sec-butoxy, tert-butoxy, isopentyloxy, isohexyloxy, and the like. Examples of cyclic alkoxy include but are not limited to cyclopropyloxy, cyclobutyloxy, cyclopentyloxy, cyclohexyloxy, and the like. An alkoxy group can include one to about 12-20 carbon atoms bonded to the oxygen atom, and can further include double or triple bonds, and can also include heteroatoms. For example, an allyloxy group is an alkoxy group within the meaning herein. A methoxyethoxy group is also an alkoxy group within the meaning herein, as is a methylenedioxy group in a context where two adjacent atoms of a structures are substituted therewith.

The terms “halo” or “halogen” or “halide” by themselves or as part of another substituent mean, unless otherwise stated, a fluorine, chlorine, bromine, or iodine atom, e.g., fluorine, chlorine, or bromine.

A “haloalkyl” group includes mono-halo alkyl groups, poly-halo alkyl groups wherein all halo atoms can be the same or different, and per-halo alkyl groups, wherein all hydrogen atoms are replaced by halogen atoms, such as fluoro. Examples of haloalkyl include trifluoromethyl, 1,1-dichloroethyl, 1,2-dichloroethyl, 1,3-dibromo-3,3-difluoropropyl, perfluorobutyl, and the like.

A “haloalkoxy” group includes mono-halo alkoxy groups, poly-halo alkoxy groups wherein all halo atoms can be the same or different, and per-halo alkoxy groups, wherein all hydrogen atoms are replaced by halogen atoms, such as fluoro. Examples of haloalkoxy include trifluoromethoxy, 1,1-dichloroethoxy, 1,2-dichloroethoxy, 1,3-dibromo-3,3-difluoropropoxy, perfluorobutoxy, and the like. The terms “aryloxy” and “arylalkoxy” refer to, respectively, an aryl group bonded to an oxygen atom and an aralkyl group bonded to the oxygen atom at the alkyl moiety. Examples include but are not limited to phenoxy, naphthyloxy, and benzyloxy.

An “acyl” group as the term is used herein refers to a group containing a carbonyl moiety wherein the group is bonded via the carbonyl carbon atom. The carbonyl carbon atom is also bonded to another carbon atom, which can be part of an alkyl, aryl, aralkyl cycloalkyl, cycloalkylalkyl, heterocyclyl, heterocyclyialkyl, heteroaryl, heteroarylalkyl group or the like. In the special case wherein the carbonyl carbon atom is bonded to a hydrogen, the group is a “formyl” group, an acyl group as the term is defined herein. An acyl group can include 0 to about 12-20 additional carbon atoms bonded to the carbonyl group. An acyl group can include double or triple bonds within the meaning herein. An acryloyl group is an example of an acyl group. An acyl group can also include heteroatoms within the meaning here. A nicotinoyl group (pyridyl-3-carbonyl) group is an example of an acyl group within the meaning herein. Other examples include acetyl, benzoyl, phenylacetyl, pyridylacetyl, cinnamoyl, and acryloyl groups and the like. When the group containing the carbon atom that is bonded to the carbonyl carbon atom contains a halogen, the group is termed a “haloacyl” group. An example is a trifluoroacetyl group.

The term “amine” includes primary, secondary, and tertiary amines having, e.g., the formula N(group)₃ wherein each group can independently be H or non-H, such as alkyl, aryl, and the like. Amines include but are not limited to R—NH₂, for example, alkylamines, arylamines, alkylarylamines; R₂NH wherein each R is independently selected, such as dialkylamines, diarylamines, aralkylamines, heterocyclylamines and the like; and R₃N wherein each R is independently selected, such as trialkylamines, dialkylarylamines, alkyldiarylamines, triarylamines, and the like. The term “amine” also includes ammonium ions as used herein.

An “amino” group is a substituent of the form —NH₂, —NHR, —NR₂, —NR₃⁺, wherein each R is independently selected, and protonated forms of each, except for NR₃⁺, which cannot be protonated. Accordingly, any compound substituted with an amino group can be viewed as an amine. An “amino group” within the meaning herein can be a primary, secondary, tertiary or quaternary amino group. An “alkylamino” group includes a monoalkylamino, dialkylamino, and trialkylamino group.

The term “amide” (or “amino”) includes C- and N-amide groups, i.e., —C(O)NR₁, and —NRC(O)R groups, respectively. Amide groups therefore include but are not limited to primary carboxamide groups (—C(O)NH₂) and formamide groups (—NHC(O)H). A “carboxamido” group is a group of the formula C(O)NR₂, wherein R can be H, alkyl, aryl, etc.

### Ole Enzymes

The α/β-hydrolase enzyme scaffold is a very common fold, used to catalyze a wide array of chemical reactions (Kazlauskas et al, 2015). The vast majority of α/β-hydrolases that have been studied initiate catalysis via attack of a catalytic nucleophile to form an acyl-enzyme intermediate that is hydrolyzed by a water molecule that is activated by a conserved histidine residue, with subsequent release of the product and a return of resting enzyme (Kazlauskas et al., 2015). Despite their biological pervasiveness, approximately 35% of enzymes annotated as α/β-hydrolases do not have a known substrate, thus their cellular function remains unknown (Kazlauskas et al., 2015).

One such α/β-hydrolase is encoded by the genes denoted as oleB, that are found in the ole (olefin) operon responsible for the biosynthesis of long-chain olefins (Sukovich, et al, 2000a; Sukovich et al., 2000b). Early studies demonstrated that long-chain olefins are generated following the head-to-head Claisen condensation of two fatty acyl-CoA molecules (Frias et al., 2011). The olefins can be 19-31 carbons in length and contain a central double bond at the site of C—C bond formation (Albro & Dittmer, 1969; Sukovich et al, 2000b; Frias et al., 2011). Genetic work in Shewanella oneidensis concretely linked the four-gene cluster, oleABCD, to hydrocarbon production (Sukovich et al., 2000a), and the ole-genes have now been identified in over 300 divergent bacteria. The α/β-hydrolase, is encoded as a stand-alone gene or as part of a gene fusion with oleC. Recently, the OleB, OleC, and OleD proteins from Xanthomonas campestris were found to associate in vivo to form an active, multi-enzyme complex when recombinantly expressed and purified from Escherichia coil, further suggesting an important function for OleB (Christenson et al., 2017b).

Until recently, only OleACD were thought to be required for the generation of long-chain olefins, leaving no apparent function for OleB (Kancharla et al., 2016). The roles of OleA and OleD as the first two pathway steps had been previously established, with OleA preforming the Claisen condensation of two acyl-CoAs to form a β-ketoacid (Frias et al., 2011; Goblirsch et al., 2016) and OleD catalyzing the NADPH-dependent reduction of the keto acid to produce a β-hydroxy acid (Bonnett, 2012). The third enzyme, OleC, was initially thought to react with the β-hydroxy acid in the presence of ATP to produce the long-chain olefin that is the endpoint of the metabolic pathway. As described herein, OleC forms a stable β-lactone under physiological conditions (see example below). In the earlier work, the OleC reaction product, the β-lactone, had been analyzed using gas chromatography at high temperature, resulting in a spontaneous decarboxylation reaction to make the observed olefin. Moreover, as described herein, while defining the chemistry of a well-known olefinic hydrocarbon biosynthesis pathway, a β-lactone synthetase was identified whose presence extends into natural product biosynthesis.

The olefin biosynthesis pathway is encoded by a four-gene cluster, oleABCD, and is found in more than 250 divergent bacteria (Sukovich et al., 2010). Ole enzymes produce long-chain hydrocarbon cis alkenes from activated fatty acids. OleA, the first enzyme of the pathway, has been studied in Xanthomonas campestris (Xc) and found to catalyze the head-to-head Claisen condensation of CoA-activated fatty acids (1) to unstable β-keto acids (2) (Frias et al., 2011). The second enzyme, OleD, couples the reduction of 2 with NADPH oxidation to yield stable β-hydroxy acids (3) as defined in Stentrophomonas maitophilia (Sm) (Bonnett et al., 2011). Finally, using gas chromatography (GC) detection methods, there are reports that Sm OleC catalyzes an apparent decarboxylative dehydration reaction to generate the final cis-olefin product (Kancharla et al., 2016).

### Exemplary Embodiments

β-Lactone Synthesis from Fatty Acyl Chains or Beta (β)-Hydroxy Acids

β-lactones may be prepared from substrates including fatty acyl chains and acyl CoA substrates using OleA, OleC and OleD. Exemplary products are shown below.

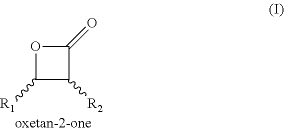

β-lactones created using OleA, OleD, and OleC include but are not limited to those where R₁ is an alkane, e.g., heptyl, nonyl, undecyl, tridecyl, or pentadecyl; unsaturated carbon chain, e.g., 10-pentadecenyl, or pentadeca-3,6,9,12-tetraenyl; methyl branched carbon chain, e.g., 14-methylpentadecyl or 13-methylpentadecyl, or a carbon chain with a hydroxy group, e.g., 2-hydroxy-4,7-dodecadienyl, and where R₂ is an alkane, e.g., hexyl, octyl, decyl, dodecyl, or tetradecyl; unsaturated carbon chain e.g., 9-tetradecenyl, or tetradec-all cis-2,5,8,11-tetraenyl, methyl branched carbon chain, e.g., 13-methyltetradecyl or 12-methyltetradecanyl, or any combination thereof. Other precursors include aikynyl, aryl, or other functional groups, which are optionally substituted. In one embodiment, a carbon atoms in a carbon may be substituted. In one embodiment, R1 or R2 independently are an alkyl or alkenyl chain that is optionally substituted, e.g., with methyl, ethyl or butyl, or a hydroxyl.

An example of the use of CoA derivative with the OleA, OleD and OleC enzymes to prepare a lactone is combing the enzymes with CoA derivatives of decanoic acid and tetradecanoic acid (myristic acid), resulting in the production of a cis-β-lactone, which can then be heated to make the cis (or Z) olefin, Z-9-tricosene.

In another embodiment, the use of CoA derivatives with OleA, NltD, and OleC enzymes to prepare trans-beta-lactones includes combining of decanoic acid and tetradcanoic acid (myristic acid) resulting in the production of a trans-beta-lactone which can be heated to make the trans (or E) olefin, E-9-tricosene.

β-lactones may be also prepared from beta-hydroxy acids, e.g., synthetically prepared beta-hydroxy acids, using OleC. In one embodiment, the β-hydroxy acid syn- and anti-diastereomers of 3-hydroxy-2-octyldecanoic acid were prepared in 50% yield following the procedure of Mulze et al. (1981). The four diastereomers were separated into syn- and anti-racemic enantiomeric mixtures by high pressure liquid chromatography. The corresponding β-lactone, 3-octyl-4-nonyloxetane-2-one, was produced trans-3-Octyl-4-nonyloxetane-2-one was isolated and purified from a mixture containing an equal mixture of the cis- and trans-β-lactones.

Exemplary , β-lactones that may be prepared from β-hydroxy acids include but are not limited to:
(+/−)-cis/trans-3-octyl-4-nonyl-oxetan-2-one,
(+/−)-cis/trans-3-octyl-4-(trans,trans-hepta-1,3-dieneyl)-oxetan-2-one,
(+/−)-cis/trans-3-(1-octynyl)-4-(1-heptynyl)-oxetan-2-one,
(+/−)-cis/trans-3-hexyl4-(2-hydroxy-cis,cis-dodeca-4,7-cis-dienyl)-oxetan-2-one, and 3-(8-phenyl-6-octynyl)- 4-heptyl-oxetan-2-one.

The drug Orlistat can be treated with aqueous NaOH which causes the β-lactone ring to open and hydrolyzes and hydrolyzes the N-formyl-L-leucine ester linkage. Treatment with OleC closes the β-lactone ring again. This suggests that OleC could be used to enzymatically treat degraded. Orlistat precursors in which β-lactone ring is opened to restore full potency. This can work with other medically-relevant β-lactones. This property can be used in manufacture (e.g., in fermentation broths or extracts), in storage, or in clinical use.

Well-known methods can be used for the synthesis of thousands of CoA derivatives from carboxylic acids (Peter et al., 2016). Since there are thousands of carboxylic acids that are commercially available, thousands of CoA esters may be combined with OleA, OleC and OleD, or OleA, OleC and LstD/NltD, to produce β-lactones.

As OleC can accept alkanes, cis-alkenes, trans-alkenes, alkynes, hydroxy alkanes, and branched alkanes as well as other substrates, a wide variety of β-lactones may be synthesized.

### Exemplary Sources for OleC, OleA and OleD

OleA, OleC, and OleD proteins can be isolated from or expressed in different sources. Host cells that may be used to express one or more of OleA, OleC or OleD, include but not limited to: Escherichia coli, Bacillus subtilis, Lactobaccillus species, Sacccharomyces cerevisiae, and many others including species in the genera Streptomyces, Kitasatospora, Saalinospora, and Nocardia. Exemplary proteins may be expressed from codon-optimized genes, e.g., for E. coli and expressed without inclusion body formation. Proteins may have a tag, e.g., His-tag to facilitate isolation. Proteins in tens of milligram quantities can be obtained from recombinant E. coli expression hosts, and purified to homogeneity in standard buffers with or without detergents. For example, standard nickel affinity chromatography may be used as described in the published papers (Christenson et al., 2017). Although other affinity chromatography techniques may be used cation exchange, anion exchange, size exclusion, affinity tag, etc). Exemplary proteins, their biological source, vectors and buffer additives are listed in Table 1 below.

TABLE 1

| | | | | Buffer |
|---|---|---|---|---|
| Protein | Organism | Accession # | Vector | Additive |
| OleA | Xanthomonas | WP_011035468.1 | pET28b⁺ | — |
| | campestris | | | |
| OleB | Xanthomonas | WP_011035472.1 | pET28b⁺ | 0.05% Triton |
| | campestris | | | X-100 |
| OleC | Xanthomonas | WP_011035474.1 | pET30b⁺ | — |
| | campestris | | | |
| OleD | Xanthomonas | WP_011035475.1 | pET28b⁺ | 0.025% |
| | campestris | | | Tween 20 |
| OleB_{D114A} | Xanthomonas | WP_011035472.1 | pET28b⁺ | 0.05% Triton |
| | campestris | | | X-100 |
| OleC | Stenotrophomonas | AFC01244.1 | pET30b⁺ | — |
| | maltophilia | | | |
| OleC | Arenimonas | WP_043804215.1 | pET30b⁺ | — |
| | malthae | | | |
| OleC | Lysobacter | WP_027070484.1 | pET30b⁺ | — |
| | Dokdonensis | | | |
| OleB-C | Micrococcus | WP_010078536.1 | pET30b⁺ | — |
| | luteus | | | |
| OleB_{D163A}-C | Micrococcus | WP_010078536.1 | pET30b⁺ | — |
| | luteus | | | |
| NltC | Nocardia | WP_042260945.1 | pET30b+ | — |
| | brasilinesis | | | |
| NltD | Nocardia | WP_04220949.1 | pET28b+ | 0.025% |
| | brasilinesis | | | Tween 20 |

OleB-C is a natural fusion of OleB and OleC in MI

OleACD genes were expressed in Escherichin coli. These enzymes are known to take fatty acyl groups from Coenzyme A or from Acyl Carrier Proteins (ACPs) and convert those into β-lactones. The β-lactones produced are known to be unstable to the heat applied in gas chromatography and decarboxylate spontaneously to the corresponding olefins. E. coli cells containing oleACD genes and the same strain lacking those genes, as a control, were extracted with an organic solvent and the extract was subjected to gas chromatography. The extract from the control strain did not show any olefins. The extract from the E coli containing oIeACD genes showed olefins of the type known to derive from β-lactones. Since the OleACD proteins are known to make those β-lactones, the E. coli likely produced those same β-lactones in vivo. The E. coli cell produced 10 different olefins, separated by gas chromatography. A recombinant, heterologously expressing cell may be engineered to produce one specific β-lactone, or like this E. coli produce a plurality, e.g., 10 or more, β-lactones that could be screened for a medically-useful activity, and then separated with chromatography. For example, E. coli recombinantly expressing OleA, OleD, and OleC employed the endogenous fatty acid pool to generate at least 10 different β-lactones including but not limited to 3-dodecyl-4-tridecyl-oxetan-2-one (mono-, di-, and likely tri-unsaturated), 3-myristoyl-4-tridecyl-oxetan-2-one (mono-, di-, and likely tri-unsaturated), 3-dodecyl4-pentadecyl-oxetan-2-one (mono, di-, and likely tri-unsaturated), and 3-myristoyl-4-pentadecyl-oxetan-2-one.

### Codon Optimized DNA Sequences for Exemplary Ole Proteins

X. campestris oleA:
(SEQ ID NO: 1)
ATGTTATTCCAAAACGTTTCTATCGCTGGTTTAGCTCACATCGATGCTCC
ACACACTTTAACTTCTAAAGAAATCAACGAACGTTTACAACCAACTTAC
GATCGTTTAGGTATCAAAACTGATGTTTTAGGTGATGTTGCTGGTATCCA
CGCTCGTCGTTTATGGGATCAAGATGTTCAAGCTTCTGATGCTGCTACTC
AAGCTGCTCGTAAAGCTTTAATCGATGCTAACATCGGTATCGAAAAAAT
CGGTTTATTAATCAACACTTCTGTTTCTCGTGATTACTTAGAACCATCTA
CTGCTTCTATCGTTTCTGGTAACTTAGGTGTTTCTGATCACTGTATGACT
TTCGATGTTGCTAACGCTTGTTTAGCTTTCATCAACGGTATGGATATCGC
TGCTCGTATGTTAGAACGTGGTGAAATCGATTACGCTTTAGTTGTTGATG
GTGAAACTGCTAACTTAGTTTACGAAAAAACTTTAGAACGTATGACTTCT
CCAGATGTTACTGAAGAAGAATTCCGTAACGAATTAGCTGCTTTAACTTT
AGGTTGTGGTGCTGCTGCTATGGTTATGGCTCGTTCTGAATTAGTTCCAG
ATGCTCCACGTTACAAAGGTGGTGTTACTCGTTCTGCTACTGAATGGAAC
AAATTATGTCGTGGTAACTTAGATCGTATGGTTACTGATACTCGTTTATT
ATTAATCGAAGGTATCAAATTAGCTCAAAAAACTTTCGTTGCTGCTAAAC
AAGTTTTAGGTTGGGCTGTTGAAGAATTAGATCAATTCGTTATCCACCAA
GTTTCTCGTCCACACACTGCTGCTTTCGTTAAATCTTTCGGTATCGATCC
AGCTAAAGTTATGACTATCTTCGGTGAACACGGTAACATCGGTCCAGCTT
CTGTTCCAATCGTTTTATCTAAATTAAAAGAATTAGGTCGTTTAAAAAAA
GGTGATCGTATCGCTTTATTAGGTATCGGTTCTGGTTTAAACTGTTCTAT
GGCTGAAGTTGTTTGG
X. campestris oleB:
(SEQ ID NO: 2)
ATGACCTACCCGGGTTATAGCTTTACGCCGAAACGCCTGGACGTCCGTC
CGGGTATTGCGATGAGCTACCTGGACGAAGGTCCGAGCGATGGCGAGGT
GGTCGTCATGCTGCACGGCAACCCGTCTTGGGGCTATCTGTGGCGTCATC
TGGTGAGCGGTCTGTCCGATCGCTACCGTTGTATCGTACCGGACCACATC
GGTATGGGTCTGTCTGACAAACCGGACGATGCGCCGGACGCACAACCAC
GTTACGATTATACTCTGCAGAGCCGTGTGGACGACCTGGACCGTCTGTTG
CAACATTTGGGCATTACCGGTCCGATTACCTTGGCAGTCCACGACTGGG
GTGGTATGATTGGCTTCGGCTGGGCCCTGAGCCATCACGCCCAAGTTAA
GCGTCTGGTTATCACCAACACGGCAGCTTTCCCGCTGCCGCCAGAGAAA
CCTATGCCGTGGCAGATTGCGATGGGTCGCCATTGGCGTTTGGGCGAGT
GGTTTATCCGCACCTTCAACGCTTTCAGCTCGGGTGCGTCTTGGCTGGGC
GTCAGCCGTCGTATGCCTGCGGCAGTGCGCCGTGCGTATGTTGCCCCATA
CGATAATTGGAAGAATCGTATTAGCACGATCCGCTTTATGCAGGATATC
CCGCTGTCCCCGGCAGATCAGGCGTGGAGCCTGCTGGAGCGTAGCGCGC
AAGCCCTGCCGTCCTTTGCAGATCGTCCGGCATTCATCGCTTGGGGTCTG
CGCGATATTTGCTTTGACAAGCATTTCCTGGCGGGTTTCCGTCGTGCGTT
GCCGCAGGCCGAAGTGATGGCGTTTGACGATGCGAACCATTACGTTCTG
GAAGATAAACATGAAGTTCTGGTTCCGGCCATCCGCGCGTTCCTGGAGC
GCAATCCGCTGTAG
X. campestris oleC:
(SEQ ID NO: 3)
ATGACTACCCTGTGCAACATCGCCGCTTCCCTGCCTCGTTTGGCCCGTGA
ACGCCCAGATCAGATTGCGATCCGTTGTCCGGGTGGCCGTGGCGCGAAC
GGCATGGCCGCATACGATGTTACCCTGAGCTACGCGGAACTGGACGCAC
GTTCTGATGCCATTGCAGCCGGTTTGGCGCTGCATGGTATTGGTCGTGGC
GTTCGCGCGGTCGTCATGGTGCGCCCGTCCCCGGAGTTCTTCCTGTTGAT
GTTCGCACTGTTCAAAGCGGGTGCGGTACCGGTTCTGGTCGATCCGGGT
ATCGACAAGCGTGCCCTGAAACAATGTCTGGACGAGGCACAGCCTCAGG
CGTTCATTGGCATTCCGCTGGCGCAGCTGGCTCGTCGTCTGCTGCGCTGG
GCTCCGTCTGCGACCCAAATTGTGACGGTCGGTGGTCGTTATTGTTGGGG
TGGTGTTACGCTGGCACGTGTCGAGCGCGATGGTGCAGGTGCAGGCAGC
CAACTGGCCGACACGGCAGCGGACGACGTGGCTGCGATTCTGTTCACGT
CGGGCAGCACCGGTGTGCCGAAAGGCGTGGTTTACCGTCACCGCCACTT
TGTTGGCCAAATCGAGCTGCTGCGTAATGCCTTCGACATGCAGCCGGGT
GGCGTAGACTTGCCGACGTTTCCTCCGTTCGCGTTGTTTGATCCGGCGCT
GGGTCTGACCAGCGTCATTCCGGACATGGATCCGACCCGTCCGGCTACC
GCAGACCCGCGTAAGCTGCATGATGCGATGACGCGCTTCGGTTTGACCC
AATTGTTCGGTAGCCCGGCACTGATGCGCGTTCTGGCGGACTACGGCCA
ACCACTGCCGAATGTTCGCCTGGCGACGAGCGCTGGTGCGCCGGTGCCG
CCAGACGTTGTCGCCAAAATTCGTGCACTGCTGCCGGCTGATGCGCAGT
TCTGGACGCCGTATGGCGCTACCGAATGCCTGCCGGTTGCGGCGATCGA
GGGTCGTACCCTGGATGCGACTCGCACCGCAACCGAAGCTGGTGCGGGT
ACCTGCGTGGGCCAGGTGGTTGCACCGAATGAGGTCCGTATCATTGCGA
TTGACGACGCGGCGATCCCGGAATGGAGCGGCGTGCGTGTGCTGGCGGC
AGGTGAGGTCGGTGAGATCACGGTGGCGGGTCCGACCACCACGGATACC
TACTTCAACCGTGATGCGGCGACCCGTAACGCTAAGATCCGTGAGCGTT
GCAGCGATGGTAGCGAACGTGTTGTGCACCGCATGGGTGACGTGGGCTA
TTTTGACGCGGAAGGTCGTCTGTGGTTTTGTGGCCGTAAGACCCATCGCG
TTGAAACTGCAACCGGTCCGCTGTATACGGAGCAGGTCGAGCCGATCTT
TAACGTGCACCCGCAGGTCCGCCGTACCGCACTGGTTGGCGTGGGCACG
CCTGGTCAGCAACAGCCGGTCCTGTGCGTTGAGTTGCAACCGGGCGTTG
CCGCGAGCGCATTTGCTGAGGTTGAAACGGCGTTGCGTGCAGTCGGTGC
AGCCCATCCACACACCGCGGGTATTGCCCGTTTTCTGCGCCACAGCGGCT
TTCCGGTGGATATCCGCCACAATGCCAAGATCGGTCGCGAAAAACTGGC
GATCTGGGCCGCACAACAACGTGTC
X. campestris oleD:
(SEQ ID NO: 4)
ATGAAAATCCTGGTTACCGGTGGTGGTGGTTTTCTGGGCCAAGCCCTGTG
TCGTGGTTTGGTCGCACGTGGTCACGAGGTTGTCAGCTTTCAGCGCGGTG
ACTACCCGGTCCTGCACACGTTGGGCGTGGGCCAAATCCGTGGTGACCT
GGCAGACCCTCAGGCGGTCCGTCACGCTTTGGCAGGTATTGATGCCGTTT
TTCACAATGCCGCCAAAGCGGGTGCATGGGGCAGCTATGATTCTTATCA
TCAAGCGAATGTCGTTGGTACTCAAAATGTCCTGGATGCGTGTCGCGCG
AACGGCGTCCCGCGTTTGATCTACACCTCCACCCCGTCGGTGACGCATCG
TGCGACGAATCCGGTTGAGGGTTTGGGTGCGGATGAAGTTCCGTACGGT
GAGGACTTGCGTGCGCCGTACGCTGCGACCAAGGCTATCGCGGAGCGTG
CGGTCCTGGCAGCCAACGACGCGCAATTGGCAACCGTTGCGCTGCGCCC
ACGCCTGATTTGGGGTCCGGGTGACAATCACCTGCTGCCGCGTCTGGCA
GCGCGTGCCCGTGCCGGTCGCCTGCGTATGGTCGGTGATGGCAGCAACC
TGGTGGACTCTACCTATATCGATAATGCAGCCCAGGCCCACTTCGATGC
GTTTGCGCACCTGGCGCCTGGTGCAGCTTGCGCGGGTAAGGCATACTTC
ATTAGCAACGGCGAACCGCTGCCGATGCGTGAGCTGCTGAACCGTCTGC
TGGCAGCGGTGGATGCCCCAGCGGTGACCCGTAGCCTGAGCTTCAAAAC
CGCGTACCGCATCGGCGCTGTGTGCGAAACCCTGTGGCCGCTGCTGCGC
CTGCCGGGTGAGGTTCCGCTGACGCGTTTCTTGGTTGAACAGCTGTGCAC
TCCGCACTGGTACAGCATGGAACCAGCACGTCGCGACTTCGGCTATGTT
CCGCAGATTTCTATCGAGGAAGGCCTGCAGCGTTTGCGTTCCAGCAGCA
GCCGCGACATTAGCATTACGCGC
X. campestris OleC
(SEQ ID NO: 5)
MTTLCNIAASLPRLARERPDQIAIRCPGGRGANGMAAYDVTLSYAELDAR
SDAIAAGLALHGIGRGVRAVVMVRPSPEFFLLMFALFKAGAVPVLVDPGI
DKRALKQCLDEAQPQAFIGIPLAQLARRLLRWARSATQIVTVGGRYGWGG
VTLARVERDGAGAGSQLADTAADDVAAILFTSGSTGVPKGVVYRHRHFVG
QIELLRNAFDMQPGGVDLPTFPPFALFDPALGLTSVIPDMDPTRPATADP
RKLHDAMTRFGVTQLFGSPALMRVLADYGQPLPNVRLATSAGAPVPPDVV
AKIRALLPADAQFWTPYGATECLPVAAIEGRTLDATRTATEAGAGTCVGQ
VVAPNEVRIIAIDDAAIPEWSGVRVLAAGEVGEITVAGPTTTDTYFNRDA
ATRNAKIRERCSDGSERVVHRMGDVGYFDAEGRLWFCGRKTHRVETATGP
LYTEQVEPIFNVHPQVRRAALVGVGTPGQQQPVLCVELQPGVAASAFAEV
ETALRAVGAAHPHTAGIARFLRHSGFPVDIRHNAKIGREKLAIWAAQQPR
X. campestris OleB D₁₁₄A:
(SEQ ID NO: 9)
ATGACCTACCCGGGTTATAGCTTTACGCCGAAACGCCTGGACGTCCGTC
CGGGTATTGCGATGAGCTACCTGGACGAAGGTCCGAGCGATGGCGAGGT
GGTCGTCATGCTGCACGGCAACCCGTCTTGGGGCTATCTGTGGCGTCATC
TGGTGAGCGGTCTGTCCGATCGCTACCGTTGTATCGTACCGGACCACATC
GGTATGGGTCTGTCTGACAAACCGGACGATGCGCCGGACGCACAACCAC
GTTACGATTATACTCTGCAGAGCCGTGTGGACGACCTGGACCGTCTGTTG
CAACATTTGGGCATTACCGGTCCGATTACCTTGGCAGTCCACGCGTGGG
GTGGTATGATTGGCTTCGGCTGGGCCCTGAGCCATCACGCCCAAGTTAA
GCGTCTGGTTATCACCAACACGGCAGCTTTCCCGCTGCCGCCAGAGAAA
CCTATGCCGTGGCAGATTGCGATGGGTCGCCATTGGCGTTTGGGCGAGT
GGTTTATCCGCACCTTCAACGCTTTCAGCTCGGGTGCGTCTTGGCTGGGC
GTCAGCCGTCGTATGCCTGCGGCAGTGCGCCGTGCGTATGTTGCCCCATA
CGATAATTGGAAGAATCGTATTAGCACGATCCGCTTTATGCAGGATATC
CCGCTGTCCCCGGCAGATCAGGCGTGGAGCCTGCTGGAGCGTAGCGCGC
AAGCCCTGCCGTCCTTTGCAGATCGTCCGGCATTCATCGCTTGGGGTCTG
CGCGATATTTGCTTTGACAAGCATTTCCTGGCGGGTTTCCGTCGTGCGTT
GCCGCAGGCCGAAGTGATGGCGTTTGACGATGCGAACCATTACGTTCTG
GAAGATAAACATGAAGTTCTGGTTCCGGCCATCCGCGCGTTCCTGGAGC
GCAATCCGCTGTAG
S. maltophilia oleC
(SEQ ID NO: 10)
ATGAATCGTCCCTGCAATATTGCGGCTCGCCTTCCCGAGCTTGCTCGCGA
ACGCCCTGACCAGATCGCGATCCGTTGCCCCGGACGTCGCGGTGCCGGA
AACGGCATGGCAGCTTATGATGTGACCTTGGATTACCGTCAATTGGACG
CGCGTAGCGACGCGATGGCAGCAGGCCTGGCTGGATACGGAATTGGGC
GTGGCGTCCGTACTGTTGTCATGGTTCGTCCCAGCCCCGAATTTTTCCTG
TTGATGTTCGCCTTGTTTAAATTAGGAGCAGTTCCTGTTCTGGTCGATCC
TGGGATTGATCGCCGCGCACTGAAGCAATGTTTGGACGAGGCTCAGCCT
GAAGCGTTTATCGGAATTCCACTGGCGCACGTAGCCCGTCTTGTTTTACG
TTGGGCGCCATCTGCGGCCCGTTTAGTTACAGTAGGGCGTCGTTTGGGCT
GGGGCGGCACTACGTTGGCTGCACTTGAGCGCGCTGGGGCGAAGGGCG
GTCCAATGCTTGCAGCAACCGACGGCGAGGATATGGCTGCCATTTTATTT
ACCTCTGGGTCAACAGGAGTACCGAAGGGGGTTGTGTATCGTCATCGCC
ACTTTGTGGGTCAAATTCAGCTTTTAGGTTCTGCGTTCGGGATGGAGGCT
GGAGGAGTCGACTTGCCTACATTTCCCCCCTTCGCTTTATTCGATCCTGC
TCTGGGGCTGACCTCGGTAATTCCCGATATGGACCCAACGCGTCCTGCTC
AGGCAGACCCTGTCCGCCTGCATGACGCTATTCAACGCTTCGGAGTCAC
ACAGCTTTTCGGTTCCCCTGCATTAATGCGTGTACTGGCTAAACATGGTC
GTCCGTTACCGACAGTGACACGTGTAACGTCAGCCGGAGCACCTGTACC
TCCCGATGTAGTAGCCACGATTCGCTCGTTGTTACCGGCGGATGCCCAGT
TTTGGACTCCGTACGGGGCTACAGAGTGTTTGCCCGTTGCAGTTGTTGAA
GGGCGTGAACTGGAGCGTACTCGCGCTGCAACTGAGGCAGGAGCGGGG
ACATGCGTTGGAAGTGTCGTAGCACCGAACGAGGTACGCATCATCGCGA
TTGACGATGCGCCTTTAGCAGACTGGTCCCAAGCCCGCGTTCTGGCTGTT
GGCGAAGTTGGGGAGATTACCGTAGCAGGCCCAACTGCTACCGATAGCT
ATTTTAATCGCCCGCAAGCAACTGCAGCCGCAAAAATCCGCGAGACCCT
TGCAGATGGTTCGACGCGCGTTGTTCATCGTATGGGCGATGTGGGGTAC
TTTGACGCTCAGGGACGCTTATGGTTCTGCGGTCGTAAAACCCAGCGCG
TTGAGACGGCGCGTGGGCCGCTGTATACAGAGCAAGTGGAGCCAGTTTT
CAATACTGTAGCAGGAGTTGCGCGTACGGCACTGGTAGGAGTTGGCGCA
GCTGGAGCCCAAGTACCAGTGTTATGTGTGGAGTTGTTGCGTGGGCAAA
GCGATAGTCCAGCCTTGCAAGAAGCGTTACGCGCGCATGCCGCAGCACG
CACCCCGGAGGCGGGTCTTCAACATTTTCTGGTCCATCCAGCGTTCCCCG
TCGACATCCGTCACAACGCCAAGATTGGGCGTGAAAAATTAGCCGTCTG
GGCGTCGGCCGAGTTAGAGAAACGTGCC
A. malthae oleC:
(SEQ ID NO: 11)
ATG TCG GAG CGC TGT AAC ATT GCG GCG GCT CTG CCA
CGC TTG GCG GCA GAA GCA CCG GAT CGC GTT GCC ATG
CGT TGT CCT GGA ACG CAT GGG GCC AAT GGC CTG GCC
CGC TAT GAC GTT GCC TTA ACG TAT GCT GGG CTT GAT
CGT CGT TCA GAT GCC ATT GCC GCA GGC CTT GCC AAA
CAC GGG GTC GCA CGT GGA CAA CGT GTT GTC GTT ATG
GTC CGT CCC TCC CCG GAA TTC TTC CTG TTA ATG TTC
GCG TTA TTT AAG GCT GGA GCC GTG CCC GTC CTT GTC
GAC CCC GGC ATT GAT AAG CGT GCC TTA AAG CAG TGT
TTA GAT GAG GCT CAG CCA CAC GCC TTT GTG GGA ATT
CCA CTT GCG ATG TTT GCG CGC AAG CTT TTA GGC TGG
GCG CGT GGA GCG AAG GTT GCG GTT ACG GTC GGT CGC
CGT TGG GCG TGG GGA GGT CCA ACT CTG GCA CAA GTC
GAG CGT GAC GGC ACT GGA GCA GGG CCG CAG CTT GCC
GAT ACA GCA CCA GAC GAA GTG GCG GCC ATC CTT TTC
ACC TCT GGC TCA ACA GGA GTG CCT AAG GGG GTT GTA
TAT CGC CAC CGT CAC TTT GTG GCA CAA ATC GAT ATG
CTT CGT GAC GCT TTT GGG CTG CAA CCA GGC GGC GTA
GAC CTG CCG ACT TTT CCA CCA TTT GCC CTT TTT GAC
CCT GCA CTG GGG TTG TCG TCG ATT ATC CCT GAC ATG
GAC CCG ACA CGC CCA GCC AAA GCC GAC CCC CGC AAG
CTG CAC GAC GCG ATT GCT CGC TTC GGA GTA GAC CAA
TTG TTT GGT TCA CCC GCT CTG ATG CGC GTG TTG GCT
GAG TAC GGT CAG CCA CTT CCG ACT TTG CGC CGT GTA
ACT AGC GCG GGA GCG CCC GTT CCG GCA GAT GTT GTT
GCT AAG ATG CGT GGG TTG TTA CCC CCC GAG GCA CAA
TTC TGG ACC CCC TAC GGG GCC ACG GAA TGC CTT CCA
GTC GCC GTG ATC GAG GCA CGC GAA CTG CAA AGC ACC
CGC GAA GCT ACA GAA CAA GGC GCT GGA ACT TGC GTA
GGA CGC CCA GTC CCC CCG AAC GAG GTA CGT ATT ATT
GCA ATC ACC GAT GCC CCG ATT GCA GAT TGG AGT CAA
GCG CAG CTG TTG GGT GCT GAA GCG ATT GGT GAA ATT
ACC GTC GCA GGC CCC AGT GCG ACG GAC GAG TAT TTT
GCT CGT CCA CAG GCG ACT GCT TTA GCT AAG ATC CGC
GAG ACG CTG CCC GAC GGC CGC CAG CGC ATC GTT CAC
CGT ATG GGA GAC CTT GGC CGT TTC GAT GCT CAA GGG
CGC TTG TGG TTC TGC GGG CGT AAA AGC CAT CGC GTT
CGC ACC CCA TTG GGT AAC CTT TAT ACG GAG CAA GTA
GAA CCT GTT TTC AAC ACA CAT CCG GAG GTT GCA CGC
ACG GCC TTG GTC GGC GTT GGA GAA GGC GCG GCG CAA
GAG CCG GTG CTG TGT GTC GAA ATG GCT CCG CAC CTG
CCT CAA TAC GAA CAC GAA CGT GTA TTA GCA GAA CTG
CGC CGC ATG TCC GAA GGA TTC GTA CAT ACT GCG CGC
ATC CGC CAT TTC CTT GTT CAT GAT GGG TTC CCT GTG
GAC ATT CGC CAT AAC GCG AAA ATT GGG CGC GAG CAA
TTG GCA GCT TGG GCC GCT AAA GAG TTG CGC TGG CGT
CGT
L. dokdonensis oleC:
(SEQ ID NO: 12)
ATG ACT GCG GCG TGT AAC ATT GCC GCA AGT CTG CCT
GCA CTG GCG CGT GCG CGC GGT GAA CAG GTA GCG ATG
CGC TGC CCG GGA CGC GAC GGT CGT TAC GAT GTG GCG
ATC ACT TAT GCT GAT TTA GAT CGT CGT TCA GAT GCG
ATT GCA GCG GGT TTG GGT AAG CGT GGT ATT GTA CGC
GGG ACT CGC ACC GTG GTT ATG GTC CGC CCC ACA CCT
GAG TTT TTT CTT TTG ATG TTT GCT CTG TTT AAA GCA
GGA GCT GTT CCT GTG TTA GTA GAC CCC GGG ATC GAC
AAA CGC GCC TTA AAG CGT TGC TTA GAC GAG GCC GAA
CCG GAT GCT TTC ATT GGG ATT CCC CTG GCC CAT TTT
GCG CGC ACG TTG CTG GGT TGG GCT CGC TCC GCA CGC
ATT CGT GTG ACT ACA GGG CGT CGC GCA CTT TTA AGC
GAC GCT ACG CTT GCC GAT GTT GAG CGT GAT GGT GCA
AAC GCC GGT CCT CAA TTA GCG GAT ACG CAG CCA GAT
GAC ATC GCG GCC ATT TTA TTC ACC TCT GGT AGC ACC
GGG GTC CCT AAA GGA GTC GTC TAC CGC CAC CGC CAT
TTC GTT GCG CAG GTA GAA ATG CTG CGC GAC GCG TTC
GGG CTG GCC CCA GGA GGC GTA GAC TTA CCG ACT TTT
CCG CCC TTC GCT CTT TTC GAT CCG GCA TTG GGA GTG
ACC AGT ATT ATC CCA GAT ATG GAT CCA ACA CGC CCA
GCG CAG GCC GAT CCA CGT CGC TTG CTT CAG GCG ATT
GAG CGT TTT GGA GTA ACC CAA TTA TTT GGT TCA CCC
GCG TTA GTG GGT GTG TTA GCA CGC CAT GGG GCA CAC
TTA CCC ACG GTA AAA CGC GTG CTG AGT GCT GGG GCT
CCC GTT CCG GCA GAC GTA GTG GCA CGT ATG CGC GAT
TTG CTT CCT GGT GAT GCT CAA TTG TGG ACG CCG TAT
GGA GCG ACC GAA TGC CTG CCT GTG TCA GTG ATT GAG
GGT CGC GAA TTG CAA TCC ACC CGT GAG GCG ACC GAG
CGT GGA GCA GGA ACG TGC GTC GGT CTG CCG GTA GCT
CCA AAT GAA GTC CGC ATC ATT CGC ATT GAC GAT GAT
GCT ATC GCT CAG TGG TCA GAT GCA CTT TTG GTC AAG
CAA GGA CAA ATT GGA GAA ATC ACG GTG GCC GGG CCC
ACT GCA ACT GAC GCG TAC TTT CGT CGT GAT GAC GCC
ACC CGC CTG GCT AAG ATT CGT GAA GCG ACT CCC GAC
GGG GAG CGT ATT GTG CAC CGC ATG GGC GAT TTG GGG
TGG ATC GAC GGC GAA GGA CGC CTG TGG TTC TGC GGC
CGT AAG ACT CAC CGC GTA GTC ATG GCA GAC GGG ACC
ACA CTT TAC ACT GAA CAG GTG GAA CCA ATT TTT AAC
GCT GCA TTC CGC GGT ATG CGT ACC GCT TTG GTT GGA
CTG GGT CCG AAA GGT GCT CAG CGT CCA GTT TTA TGT
TAC GAG GTG CCT AAA GAC GTC GGA CAC AAT GCT GCT
GAT CTG CCT GGG GAA TTG CGC CAT TTT GCC GAA GGA
CGC GTG CAC ACT GCG AAA ATT CAC CAT TTT TTG CCC
CAC CCT GGG TTC CCG GTA GAC ATC CGT CAT AAC GCG
AAA ATT GGG CGC GAG AAA TTA GCA GCG TGG GCG ACG
CGC CAA TTA GAA AAA CGC GCA
M. luteus oleBC fusion:
(SEQ ID NO: 13)
ATGCCGCAGATTCCAGCCGCTCCAGCCGCCCTTCCACCTGCCGATCGTCT
GCCGGGTTGGGACCCAGCTTGGAGCCGTCTGGTCGAAATCCGTTCCGCA
GCGGATCCGGAAGGTACCGTCCGTACGCTGCATGTCGCCGATACCGGTC
CGGTCCTGGCGGCAGCGGGTGCAGAGATTGTTGGTACGATCGTTGCAGT
TCATGGTAATCCGACGTGGTCTTGGCTGTGGCGCAGCCTGCTGGCAGAG
ACTGTCCGTCGTGCGCGTCGTGGTATGGCGGCTTGGCGTGTCGTTGCGCC
GGATCAGCTGGACATGGGTTTCTCCGAACGTCTGGCGCACGCTGGTAGC
CCTAGCGCAGCATCGATGGGCCGTGCGGGTGACACGTATCGTACCCTGG
GTGGCCGCATCGCAGATCTGGACGCACTGCTGACTGCCCTGGGTCTGCG
CGATCTGGCCGCGACCGGTCATCCACTGATCACCCTGGGCCACGACTGG
GGCGGTGTTGTTAGCCTGGGTTGGGCAGCTCGTCATCCGGAGCTGGTCG
CGGGTGTGGCGACGCTGAACACCGCGGTCCACCAACCGGAAGGTGCGCC
AATTCCGGCACCGCTGCAACCAGCGTTGGCGGGTCCTGTGCTGCCGGCA
TCCACGGTTACCACCGACGCATTTCTGTCCGTCACCACCTCGCTGGCCAC
CCCGGCTTTGGACCGTGAAACCCGTGCCGCTTACCATCTGCCGTACGAC
ACGGCGGCACGTCGTGGCGGCGTTGGTGGTTTTGTCGCAGACATTCCGG
CGGACCCTGGCCACGGTAGCCACCCGGAGCTGCAGCGCGTTGGTGAAGA
TCTGGCGGCACTGGGTCGTACCGACGTTCCAGCGCTGATTCTGTGGGGT
GCTGACGACCCCGTTTTTCTGGACCGCTACTTGGACGATCTGCGTGATCG
CCTGCCGCATGCCCGTGTCCACCGTTATGAGCGCGCAGGCCATCTGCTG
GTTGACGACCGCGATATCACCGCTCCGCTGCTGCAATGGGCGCAGTTGC
TGCGCGGTGGTCAATTGTCTGACCCAGCATCGGGTTTGCCCGGTCCGGT
GCCTCACGCGACTGCCGATGCAGCCGCAGATCCGGGTCTGGAAGTGGAC
CTGGGCGAGGACCCGGGTGCCCGTGAGCCGGGTGTTGTTCGTTTGTGGG
ATCACTTGCGTGATTGGGGTGCGCCAGGCAGCGATCACCGTGAGTATAC
GGCGCTGGTGGATATGGCGGGTGCGCAGGCTGGCCGCAGCTTGGTCGGC
ACCGCACGCCGTCCGGTAGCGGTCACGTGGGGTGAGCTGCAAGAAATGG
TTTCCGCGATTCCAACCGGCCTGTGGGCTGCTGCTATGCGTCCGGGCGA
CCGTGTGGCTATGCTGGTTCCGCCTGGTCGTGATCTGAGCGCGGCATTGT
ACGCAGTGCTGCGCGTTGGCGCCGTCGCTGTTGTTGCGGATCAAGGTCT
GGGTGTGAAAGGTATGACCCGTGCGATGAAGAGCGCACGTCCTCGCTGG
ATTATTGGTCGCACGCCGGGTCTGACGCTGGCTCGTGCGCAATCGTGGC
CTGGCACGCGTATCAGCGTGACCGAGCCAGGTGCGGCGCAGCGCCGTCT
GCTGGACGTGAGCGACAGCCTGTATGCAATGGTTGACCGTCATCGCGAT
CCGGCAGCAGGCGATGCGGTCGACGAGCATGGTACGGTCCTGCCTGAGC
CGGCACTGGATGCAGATGCGGCAGTCCTGTTCACGAGCGGTTCTACGGG
TCCGGCCAAGGGTGTGGTGTACACTCACGAGCGTTTGGGCCGCTTGGTT
GCACTGATCAGCCGCACCCTGGGTATCCGTCCGGGTGGTAGCCTGCTGG
CCGGTTTCGCACCGTTCGCGCTGTTGGGCCCAGCACTGGGTGCCGCGTCC
GTTAGCCCGGACATGGATGTGACCCAACCGGCAACCCTGACGGCCCAAA
AGCTGGCCGACGCGGCCATTGCGGGTCAAAGCAGCGTGCTGTTTGCTAG
CCCGGCAGCGCTGGCAAACGTGGTGGCAACTGCAGACGGTCTGGATGCA
CCGCAGCGTGAGGCGTTGGACGCGGTGCGTCTGGTGCTGAGCGCCGGTG
CACCGGTTCACCCGCAGCTGATGCGCCAAGTTAGCGACCTGATGCCGAA
CGCGCGTGTCCACACCCCGTGGGGCATGACCGAAGGTCTGCTGCTGACC
GATATCGATGGTGATGAAGTCCAGCGCCTGCGTACGGCCGATGATGCGG
GCGTCTGCGTGGGTAGCGCGCTGCCGACGGTGTCTCTGGCGATCGCACC
GCTGTTGGAAGATGGTAGCGCGGAAGATGTCATTCTGGATCCGGCACGC
GGTCACGGCGTCTTGGGCGAGATTGTCGTTAGCGCACCGCACCTGAAGG
ACCGTTACGACGCGCTGTGGCATACGGACCAGCAGAGCAAGCGTGACG
GTCTGTGGCGCCGTGATGGCCGTGTGTGGCACCGTACGGCGGATGTTGG
TCATTTCGATGCCGAAGGTCGTGTTTGGCTGGAAGGTCGCCTGCAGCAC
GTGATCACCACGCCGGAAGGTCCTGTCGGTCCTGGTGGTCCGGAGAAAA
CCGTTGATGCGCTGGGTCCGGTTCGTCGTAGCGCCGTTGTCGGTGTTGGC
CCTCGCGGTACCCAAGCGGTTGTTGTCGTTGTTGAAGCAGCAGTTCCGGC
TACCCGTCCGGCTCGTCGTCCTGGTCACCATCGCGATGGCCGTCCGAAAC
AGGGCTTGGCGCCGACCGCCTTGGCATCGGCGGTGCGTGCTGCGCTGGA
GCCGCTGCCGGTCGCTGCGGTTTTGGTTGCTGACGAGATTCCGACCGAC
ATTCGTCACAATTCTAAAATCGACCGTGCCCGTGTTGCAGATTGGGCCG
AAGCGGTTCTGGCCGGTGGCAAAGTTGGTGCGCTGCA
M. luteus oleBC fusion D₁₆₃A (in OleB domain):
(SEQ ID NO: 14)
ATGCCGCAGATTCCAGCCGCTCCAGCCGCCCTTCCACCTGCCGATCGTCT
GCCGGGTTGGGACCCAGCTTGGAGCCGTCTGGTCGAAATCCGTTCCGCA
GCGGATCCGGAAGGTACCGTCCGTACGCTGCATGTCGCCGATACCGGTC
CGGTCCTGGCGGCAGCGGGTGCAGAGATTGTTGGTACGATCGTTGCAGT
TCATGGTAATCCGACGTGGTCTTGGCTGTGGCGCAGCCTGCTGGCAGAG
ACTGTCCGTCGTGCGCGTCGTGGTATGGCGGCTTGGCGTGTCGTTGCGCC
GGATCAGCTGGACATGGGTTTCTCCGAACGTCTGGCGCACGCTGGTAGC
CCTAGCGCAGCATCGATGGGCCGTGCGGGTGACACGTATCGTACCCTGG
GTGGCCGCATCGCAGATCTGGACGCACTGCTGACTGCCCTGGGTCTGCG
CGATCTGGCCGCGACCGGTCATCCACTGATCACCCTGGGCCACGCGTGG
GGCGGTGTTGTTAGCCTGGGTTGGGCAGCTCGTCATCCGGAGCTGGTCG
CGGGTGTGGCGACGCTGAACACCGCGGTCCACCAACCGGAAGGTGCGCC
AATTCCGGCACCGCTGCAAGCAGCGTTGGCGGGTCCTGTGCTGCCGGCA
TCCACGGTTACCACCGACGCATTTCTGTCCGTCACCACCTCGCTGGCCAC
CCCGGCTTTGGACCGTGAAACCCGTGCCGCTTACCATCTGCCGTACGAC
ACGGCGGCACGTCGTGGCGGCGTTGGTGGTTTTGTCGCAGACATTCCGG
CGGACCCTGGCCACGGTAGCCACCCGGAGCTGCAGCGCGTTGGTGAAGA
TCTGGCGGCACTGGGTCGTACCGACGTTCCAGCGCTGATTCTGTGGGGT
GCTGACGACCCGGTTTTTCTGGACCGCTACTTGGACGATCTGCGTGATCG
CCTGCCGCATGCCCGTGTCCACCGTTATGAGCGCGCAGGCCATCTGCTG
GTTGACGACCGCGATATCACCGCTCCGCTGCTGCAATGGGCGCAGTTGC
TGCGCGGTGGTCAATTGTCTGACCCAGCATCGGGTTTGCCGGGTCCGGT
GCCTCACGCGACTGCCGATGCAGCCGCAGATCCGGGTCTGGAAGTGGAC
CTGGGCGAGGACCCGGGTGCCCGTGAGCCGGGTGTTGTTCGTTTGTGGG
ATCACTTGCGTGATTGGGGTGCGCCAGGCAGCGATCACCGTGAGTATAC
GGCGCTGGTGGATATGGCGGGTGCGCAGGCTGGCCGCAGCTTGGTCGGC
ACCGCACGCCGTCCGGTAGCGGTCACGTGGGGTGAGCTGCAAGAAATGG
TTTCCGCGATTGCAACCGGCCTGTGGGCTGCTGGTATGCGTCCGGGCGA
CCGTGTGGCTATGCTGGTTCCGCCTGGTCGTGATCTGAGCGCGGCATTGT
ACGCAGTGCTGCGCGTTGGCGCCGTCGCTGTTGTTGCGGATCAAGGTCT
GGGTGTGAAAGGTATGACCCGTGCGATGAAGAGCGCACGTCCTCGCTGG
ATTATTGGTCGCACGCCGGGTCTGACGCTGGCTCGTGCGCAATCGTGGC
CTGGCACGCGTATCAGCGTGACCGAGCCAGGTGCGGCGCAGCGCCGTCT
GCTGGACGTGAGCGACAGCCTGTATGCAATGGTTGACCGTCATCGCGAT
CCGGCAGCAGGCGATGCGGTCGACGAGCATGGTACGGTCCTGCCTGAGC
CGGCACTGGATGCAGATGCGGCAGTCCTGTTCACGAGCGGTTCTACGGG
TCCGGCCAAGGGTGTGGTGTACACTCACGAGCGTTTGGGCCGCTTGGTT
GCACTGATCAGCCGCACCCTGGGTATCCGTCCGGGTGGTAGCCTGCTGG
CCGGTTTCGCACCGTTCGCGCTGTTGGGCCCAGCACTGGGTGCCGCGTCC
GTTAGCCCGGACATGGATGTGACCCAACCGGCAACCCTGACGGCCCAAA
AGCTGGCCGACGCGGCCATTGCGGGTCAAAGCAGCGTGCTGTTTGCTAG
CCCGGCAGCGCTGGCAAACGTGGTGGCAACTGCAGACGGTCTGGATGCA
CCGCAGCGTGAGGCGTTGGACGCGGTGCGTCTGGTGCTGAGCGCCGGTG
CACCGGTTCACCCGCAGCTGATGCGCCAAGTTAGCGACCTGATGCCGAA
CGCGCGTGTCCACACCCCGTGGGGCATGACCGAAGGTCTGCTGCTGACC
GATATCGATGGTGATGAAGTCCAGCGCCTGCGTACGGCCGATGATGCGG
GCGTCTGCGTGGGTAGCGCGCTGCCGACGGTGTCTCTGGCGATCGCACC
GCTGTTGGAAGATGGTAGCGCGGAAGATGTCATTCTGGATCCGGCACGC
GGTCACGGCGTCTTGGGCGAGATTGTCGTTAGCGCACCGCACCTGAAGG
ACCGTTACGACGCGCTGTGGCATACGGACCAGCAGAGCAAGCGTGACG
GTCTGTGGCGCCGTGATGGCCGTGTGTGGCACCGTACGGCGGATGTTGG
TCATTTCGATGCCGAAGGTCGTGTTTGGCTGGAAGGTCGCCTGCAGCAC
GTGATCACCACGCCGGAAGGTCCTGTCGGTCCTGGTGGTCCGGAGAAAA
CCGTTGATGCGCTGGGTCCGGTTCGTCGTAGCGCCGTTGTCGGTGTTGGC
CCTCGCGGTACCCAAGCGGTTGTTGTCGTTGTTGAAGCAGCAGTTCCGGC
TACCCGTCCGGCTCGTCGTCCTGGTCACCATCGCGATGGCCGTCCGAAAC
AGGGCTTGGCGCCGACCGCCTTGGCATCGGCGGTGCGTGCTGCGCTGGA
GCCGCTGCCGGTCGCTGCGGTTTTGGTTGCTGACGAGATTCCGACCGAC
ATTCGTCACAATTCTAAAATCGACCGTGCCCGTGTTGCAGATTGGGCCG.
AAGCGGTTCTGGCCGGTGGCAAAGTTGGTGCGCTGCA

### An Assay to Identify β-Lactone Synthetases In Vitro and In Vivo

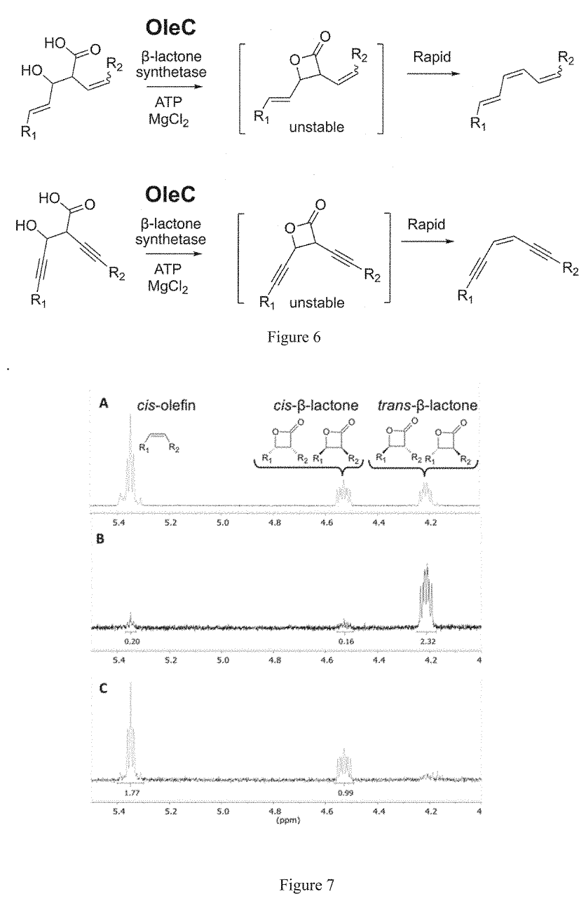

The assay employs a β-lactone synthetase substrate having two conjugated C═C bonds, or two acetylenic groups, conjugated with the produced β-lactone. The β-lactone is so unstable as to spontaneously decarboxylate at room temperature and pH 7, thus forming a triene with a very high extinction coefficient that can readily be detected spectrophotometricaily in a cuvette or in a micro-titer well plate. Another comparable substrate with two triple bonds (see below) in resonance reacts similarly. See FIG. 6.

The above reactions can he used to identify and measure the activity of β-lactone synthetases. The substrates (on the left) are not observable in the UV spectrum regions indicated, however the product shows a very strong absorbance. This is useful to screen enzymatic activity in vitro and in vivo and is amenable to a high-throughput screening method in microtiter well plates.

Assays were run in 20 mM NaPO₄, 200 mM NaCl, 2% ethanol (for substrate solubility) at pH 7.4 at room temperature (see Robinson et al., Chem Bio Chem (2019), the disclosure of which is incorporated by reference herein).

### Assay Method 1

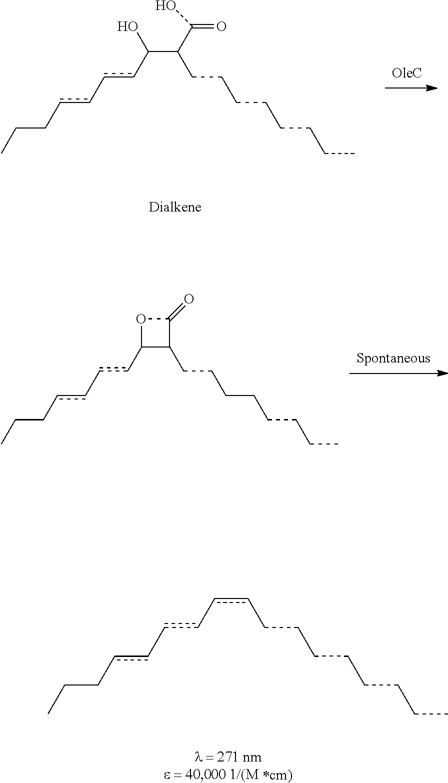

Assay Method 2

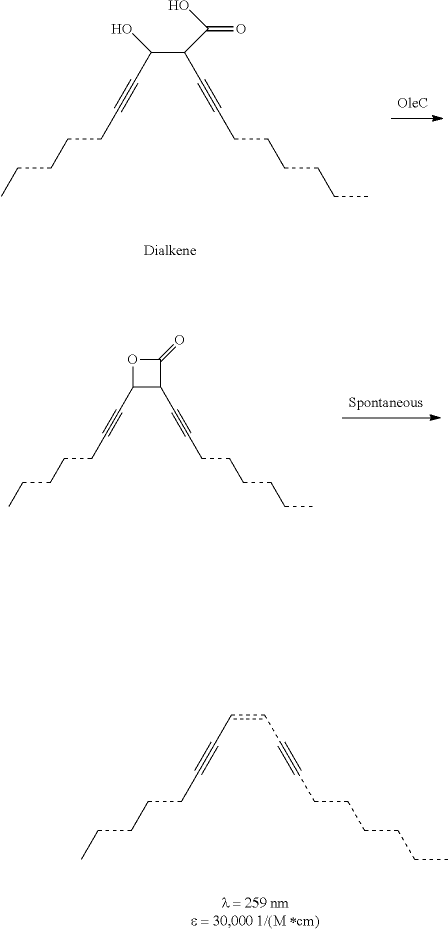

Assay Method 3

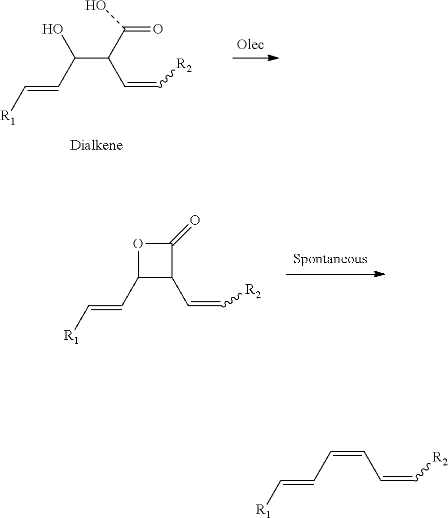

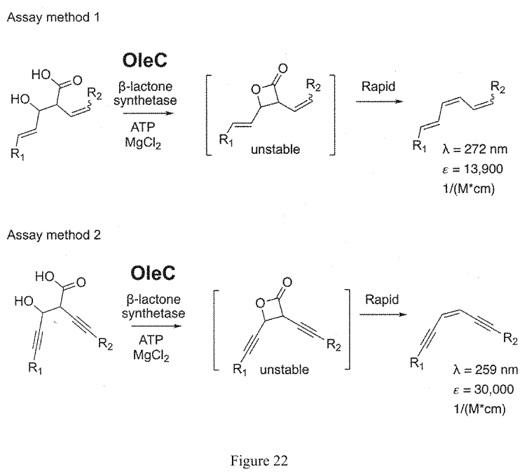

See also FIG. 22.

In one embodiment, R₁ and/or R₂ have tails with more than one alkenyl or alkynyl group. In one embodiment, R₁ and R₂ independently is alkyl, alkenyl, alkynyl, or aryl which is optionally substituted, e.g., with groups including hydroxyl. In one embodiment, R₁ and R₂ independently are C6-C14. In one embodiment, R1 and R2 independently are C2-C6. In one embodiment, R₁ and R₂ independently are C6-C10. In one embodiment, R₁ and R₂ independently are C8-C12. In one embodiment, R₁ and R2 independently are C10-C14.

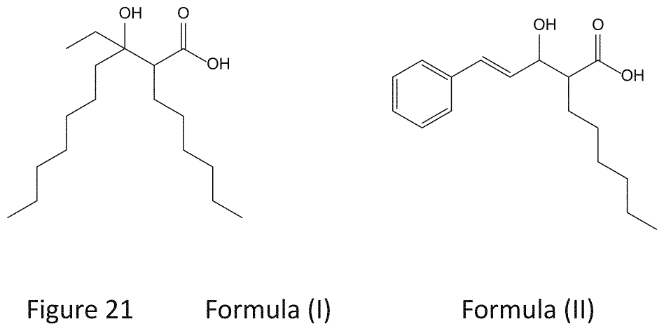

In one embodiment, the substrate comprises a substrate with three, four, or more double bonds in conjugation. The more double bonds the longer the wavelength and so the more readily detectable the compound becomes. In one embodiment, the substrate is formula (II) in FIG. 21.

### Use of Ole Enzymes in Bioremediation

Bioremediation typically removes a waste product from a given environment and is a net cost to industry because it does not contribute to making more saleable product. For example, fast food restaurants in the U.S. generate 4,793,137 gallons of grease waste weekly, Waste grease is a problem for these restaurants, and bioremediation by bacteria and enzymes are used to clear their clogged drains.

Greases are triacylglycerides that are biodegraded to glycerol, which is readily metabolized by most bacteria for energy, and fatty acids. The fatty acids are metabolized to β-lactones. Since waste greases consist of complex mixtures of fatty acyl chains that come together in different combinations, it is possible to generate wmore than 1,000 different β-lactones using Ole enzymes since the enzymes have very broad specificity. Increasing the fatty acid pool increases the number of β-lactones produced. A broad specificity triacylglycerol hydrolase that releases many fatty acids from gwwreases may be used in combination with Ole enzymes to produce hundreds, or even thousands of fβ-lactones.

A Method for Making Cis- or Trans-β-Lactones using β-Lactone Synthetase and Purified Diastereomers of β-Hydroxy Acid Precursors that Selectively give Cis- or Trans-β-Lactones

Most therapeutic β-lactones that are approved or in trials are trans-β lactones. OleC (β lactone synthetase) can make cis or trans lactones. The configuration OleC forms depends on the stereochemistry of the β-hydroxy acid substrate. Since OleA and OleD proteins feed in hydroxy acids that result in cis-βlactones, to make trans-lactones, synthetic β-hydroxy acids may be employed.

Alternatively, OleA and NltD proteins can be combined to make trans-beta lactones.

High-performance liquid chromatography (HPLC) can e used to separate syn and anti diastereomer pairs. A synthesized mixture of syn- and anti-2-octyl-3-hydroxydodecanoic acid was separated into the syn and anti diastereomer pairs by HPLC (Hewlett Packard) using a reverse phase C18 column (Agilent eclipse plus 4.6×250 mm). Sample was dissolved at 2.0 mg/mL in acetonitrile (ACN, Sigma) containing 4 mM HCl. Column was prewashed with 100% acetonitrile and 40% methyl tert-butyl ether (MTBE, Sigma) prior to 100 μL sample injection. The program was as follows: hold 100% ACN for 2 min; ramp MTBE to 40% by 10 min; hold MTBE 40% to 15 min; back to 100% ACN until 18 min. Detection wavelength was set to 220 nm. Fractions were manually collected from 10 runs to accumulate approximately 1.0 mg of each diastereomer pair.

Moreover, LstA (as a heterodimer with LstB) in the lipstatin pathway, and NltA and NltB, may be used to replace OleA to form 2R-β-keto acid of the opposite configuration of the 2S-β-keto acid produced by the Ole pathway. Additionally, LstD in the lipstatin biosynthetic pathway or NltD in the nocardiolactone biosynthetic pathway may catalyze the same reaction as OleD but may act to produce β-hydroxy acids that form trans-β-lactones, because the natural product lipstatin has a trans configuration in the β-lactone ring. Thus, LstA and LstB or NltA and NltB may replace OleA, and LstD or NltD may replace OleD.

In another embodiment, acyl-CoA substrates are employed with LstA, LstD and OleC to prepare trans-β-lactones. Exemplary LstA and LstD polypeptides are:

LstA
(SEQ ID NO: 15)
MSTTERRSRIEALGAFLPAGRETNDELRAKVPNLGDADVRRITGIAERRV
HDPDPAAGEDSFGMALAAARDCLAVSRHRAADLDVVISASITRVKDGSRF
HFEPSFAGMLAKELGARPAISFDVSNACAGMMTGVWLLDRMIRSGAVRSG
MVVSGEQATRVARTAARELRDSYDPQFASLSVGDSAAAVVLDESTDPADR
IHYIELMTCAAYSHLCLGMPSDRSQGIGLYTDNKKMHDRERLKLWPRFHE
DFLAKNGRRFEDEEFDHIIQHQVGTRFIEYANRTAEAEFAAPMPPSLQVV
EQYGNTATTSHFLTLRDHLRRTRGAGATGTGTGPGSGPGAGPAREAAGAK
YLLVPAASGLVTGALSATVTHAGA
LstB
(SEQ ID NO: 26)
AIT38299.1 LstB [Streptomyces toxytricini]
MGIVITASATATHTDPGTPASAVDLAGRAARRCLAHARVSPSGVGVLVNV
GVYRENNTFEPALAALVQKETGINPDYLADPQPAAGFSFDLMDGACGVLS
AVQAGQSLLSTGTTERLLITAADVHPGGDASRDPDYPYADLAGAFLLERD
ADPDTGFGPVRHYGGGDRPTDVAGYLDLDTMGSGGRSRITVHRTPGHEQR
TGELAAAAYAAYTGEFGLDAGRTLVIGPDAPAGVGDGPGGGRPHTAAPVL
GYLHALESARPEGVDTLLFVTAGAGPRAAVASYRPQGW
LstD
(SEQ ID NO: 16)
MKILITGATGFLGGHLADACLRSGHGVRALVRPGSNTDRLRALPGVELVT
GDLTRPDSLRRAADGCEAVLHSAARVVDHGTRAQFTEANVTGTLRLMDAA
RAAGVRRFVFVSSPSALMHLREGDRLGIDETTPYPTRWFNDYCATKAVAE
QHVLAADTAGFTTCALRPRGIWGPRDHAGFLPRLIGALHAGRLPDLSGGK
HVLVSLCHVDNAVDACLRAAVSAPAERIGGRAYFVADAETTDLWPFLADV
AARLGCPPPAPRIPLPAGRALAAAVETAWRLRPDAAARARSSPPLSRYMM
ALLTRSSTYDTTAARRDLGYTPVRTQEDGLRDLVRWVASQGGVASWTAPR
PHPAHTHTPDATPHAPARAPHPPMPEPPAAATPAPPPKAEHRPALPRPRS
SPEADSTEQPFPHPADATDTPPVSGPAPGPVSVPAPDRTPAPSGSSRTAG
DAPACRAGQASGPAPAPVRGPADARSAATGRGPRPVRGSAEQREHRDPSL
RASGKPGSDGSGAPADTRPNHDPTRAEAARPGDAGRGMAPEGDTARRGST
DPAGPAGREDTSR

See below for a discussion of Nlt enzymes and uses therefor.

### ATP Regenerating Systems

Since OleC requires ATP, ATP may be supplied as an ATP-regenerating system to continuously recycle ATP→ADP→ATP (Zhao and van der Donk, 2003). A very common method of generating ATP is the use of a high energy phosphate compound such as phosphoenoyl pyruvate (PEP), ADP and an enzyme to transfer the phosphate group to ADP to generate ATP. The regeneration of AMP to ATP is commonly done in two ways. The first method requires two enzymes. The first enzyme, such as adenosine-5′-monophosphate kinase, converts an ATP and AMP into two ADPs (di-). A second enzyme, such as acetate kinase, uses commercially available acetyl phosphate as an energy source to convert the ADP to ATP releasing acetate. The second method converts AMP to ATP directly by providing commercially available polyphosphate to a polyphosphate kinase 2 class III enzyme. See also Andex and Richter, Chem Bio Chem., 16:380 (2015), the disclosure of which is incorporated by reference herein.

A Method for Making β-Lactones with Ole Enzymes Allowing for Racemization of the OleA Product to Occur so as to Increase the Preponderance of Trans-β-Lactones

A previous study assayed OleD by measuring NADP+ reduction and the oxidation of β-hydroxy acids, the opposite direction of the physiologically relevant reaction (Bonnett, et al, 2011). All four diastereomers were oxidized, but the kinetically favored diastereomer was the 2S,3R-β-hydroxy acid, suggesting that OleA initially forms a 2S-β-keto acid product. However, even if OleA shows complete enantiospecificity, a β-keto acid might undergo keto-enol tautomerization between the C-2 and C-3 carbon atoms such that one might expect to see racemization of the stereochemistry.

The syn- and anti-β-hydroxy acid intermediates give rise to cis- and trans-β-lactones, respectively, by OleC. The OleA-catalyzed reaction was employed to produce the β-keto acid and then a protein mixture composed of OleD and OleC was added at different time intervals. With OleACD co-incubated from time zero, there was evidence for only a minor amount of trans-β-lactone formation in the major cis-β-lactone product mix. However, at short time intervals of several minutes, and with increasing time, significant and increasing levels of E-olefins were observed by gas chromatography analysis of the product mix. This is consistent with the scrambling of the stereochemistry at C-2 and the formation of 2S,3R (syn) and 2R,3R (anti) as the major diastereomers formed, based on the previous reports of the stereochemical preferences of OleD. These diasteromers give rise to cis- and trans-β-lactones.

To more directly demonstrate keto-enol tautomerization, reactions producing the β-keto acid in deuterated water, D₂O, were conducted. Deuterium would only be expected in the β-hydroxy acid from keto-enol tautomerization because hydride transfer from NADPH by OleD would not introduce deuterium during the reduction step. The reactions were run with OleA, OleD, NADPH and D₂O, and reactions were quenched by organic solvent with methylation of the carboxyl group using diazomethane. The methylated β-hydroxy acid products were analyzed by GC-MS.

Significant and increasing deuterium incorporation was observed over time.

Thus, the prevalence of trans-β-lactones can be increased by incubating an acyl-CoA with OleA in the absence of other enzymes, allowing spontaneous stereochemical scrambling to occur, and then adding OleD and OleC to make a mixture of cis- and trans-β-lactones. These reactions may be conducted in the same reaction vessel e.g. a well in a 96-well microplate. The reaction of OleA is very thermodynamically favorable and is therefore irreversible. The OleA reaction does not have to be complete. As soon as the first molecules of OleA product are created (β-ketoacid) OleD and OleC can convert the product to a β-lactone while there is still substrate present for OleA. If OleD and OleC are not added to the reaction vessel for a time, different ratios of the R and S configurations of the β-lactone are obtained by keto-enol tautomerization.

A Method for Enriching Trans-β-Lactones, which are more Frequently Found in Medicinal Natural Products, from a 1:1 Mixture of Cis- and Trans-β-Lactones.

This method employs the enzyme OleB, β-lactone decarboxylase, that acts on a cis-β-lactones and has no detectable activity with trans-β-lactones, thus leaving behind trans-β-lactones when contacting a cis- and trans-mixture. Note that this method does not leave pure trans-β-lactones as only one of two diastereomers of a cis-β-lactone mixture is decarboxylated by a β-lactone decarboxylase.

To determine if OleB catalyzes the terminal reaction in long-chain olefin biosynthesis, it was necessary to synthesize β-lactones containing two hydrocarbons tails in the range of C₈-C₁₄. Those chain lengths were previously shown to be in the biologically relevant range. Both cis- and trans-3-octyl-4-nonyl-2-oxetanone (cis- and trans-β-lactone) were chemically synthesized here and used to determine if OleB catalyzes a decarboxylation reaction. ¹H-NMR demonstrated that both the cis- and trans-β-lactones enantiomeric pairs contained <10% of the opposite configuration. ¹H-NMR analysis of OleB reactions showed that 47% of the cis- β-lactone underwent decarboxylation to the cis-olefin in a long-term reaction that went to completion. These results suggest that OleB selectively acts on only one of the cis-β-lactone enantiomers. It is presumed that OleC maintains the 2R,3S stereo-centers confirmed in the product of OleD, and therefore is likely that OleB acts on the 2R,3S cis-β-lactone.

An OleB reaction mixture showed only 4% of the trans-β-lactone underwent decarboxylation, and the product was a cis-olefin. This 4% product is likely caused by the small contamination of cis-β-lactone in the trans-β-lactone sample. Olefin was undetectable in control reactions lacking enzyme. A synthetic trans-olefin standard was prepared to aid in analytical methods, but there was no evidence for this compound being produced in OleB reaction mixtures. This observation agrees with multiple literature reports that the bacteria examined produce cis-olefins exclusively (Albro & Dittmer, 1969; Sukovich et al., 2000b). Taken together, these data supported the idea that OleB acts physiologically to catalyze decarboxylation of cis-β-lactones to yield cis-olefins that complete the olefin biosynthetic pathway.

Assays for Lactones that Modulate Lipases, Proteosomes, Penicillin-Binding Proteins, Bacterial or Cancer Cells

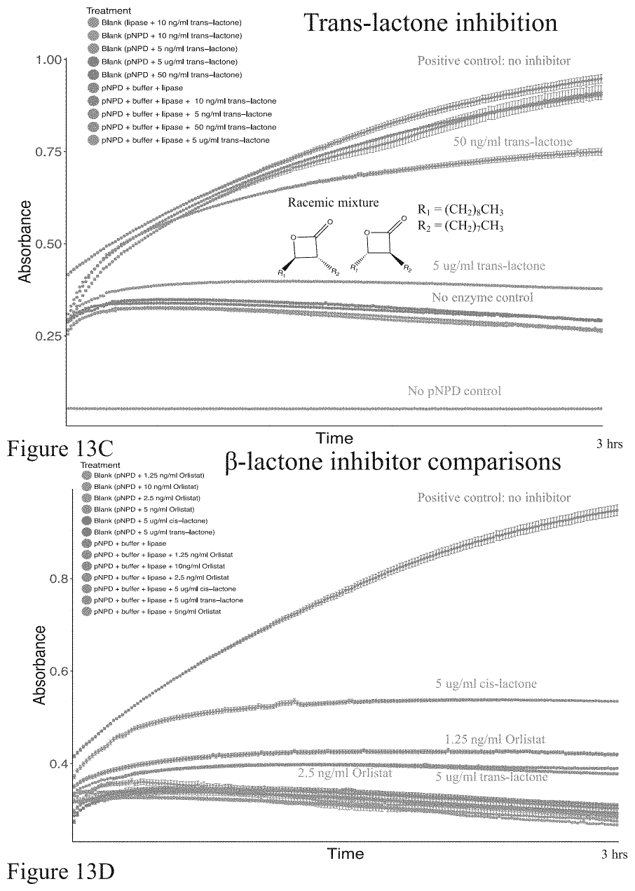

Orlistat, an anti-obesity drug with a β-lactone moiety, inhibits pancreatic lipase at nanogram levels (see FIG. 13A). Cis and trans-isomers of β-lactones were synthesized and tested separately. As expected, the trans-isomer had higher lipase inhibitory activity than cis- (FIGS. 13B-D), e.g., at microgram levels. A β-hydroxy acid (synthetic) was combined with and OleC and lipase and comparable inhibition was observed (FIG. 13E). These reactions were conducted in microtiter well plates so the inhibition of the lipase can be measured in a high-throughput manner such that hundreds or thousands of β-lactones can be rapidly screened. In addition, fatty acyl-CoA substrates can be combined with OleADC and lipase and lipase inhibition can be detected. Moreover, lipase may be substituted with proteasomes, penicillin binding proteins, pathogenic bacteria, or cancer cells and inhibitory activity of one or a plurality of β-lactones detected.

Use of an OleABCD System, In Vitro or In Vivo, in which OleB (a β-Lactone Decarboxylase that Destroys β-Lactones) is Mutated

OleB is the final enzyme of the biosynthesis pathway to olefins, and decarboxylates cis-β-lactones to cis-olefins. OleBCD forms a complex that may help create olefins efficiently in a native organism. However, mutating OleB to prevent function leads to the accumulation of β-lactones. By mutating aspartate-114 in OleB to an alanine-114 using site-directed methodologies, OleABCD may be expressed together to make β-lactones instead of cis-olefins. Aspartate-114 is essential for catalysis so its mutation to alanine renders the resultant OleB completely inactive. This OleA+-OleB₍ₘᵤₜ₎CD complex may be more efficient than OIeA+OIeD+OIeC.

A Combinatorial Method using Mixtures of Enzymes from Different Sources to make Large Numbers of β-Lactones in One-Pot for Large Scale Combinatorial Screening

While many of the experiments described herein use the Ole proteins from Xanthomonas campestris, there are at least 300 likely more than 600 different organisms that contain OleC homologs. Besides Xanthomonas campestris, OleC proteins from Stentrophomonas maltophilia, Arenimonous malthae, Lysobacter dokdonensis and Micrococcus luteus have been tested. While the sequence identity extends to as low as 35%, all homologs tested were found to make β-lactones. Preliminary evidence suggests that different enzymes have different substrate specificities. This indicates that many structurally different β-lactones can be made.

TABLE 2

| Organism | Accession # | % ID^{a} |
|---|---|---|
| Xanthomonas campestris | WP_011035474.1 | 100 |
| Stenotrophomonas | AFC01244.1 | 77 |
| maltophilia | | |
| Arenimonas malthae | WP_043804215.1 | 73 |
| Lysobacter dokdonensis | WP_036166093.1 | 70 |
| Micrococcus luteus | WP_010078536.1 | 35 |

^{a}% identity based on amino acid sequence

Since there are minimally 300 each of OleA, OleC, and OleD proteins known, there are 300×300×300=27 million different combinations of proteins that can allow for a broad array of potential β-lactones to be produced.

Bionformatic Methods for Identifying oleC Genes and β-Lactone Biosynthetic Gene Clusters in Genomic and Metagenomic DNA Sequences or in Gene Repositories

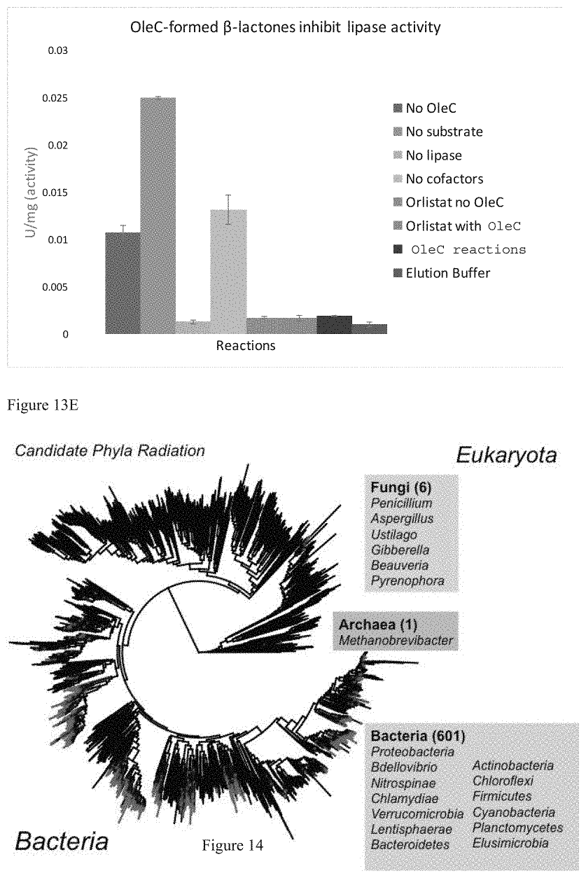

A bioinformatics pipeline was developed to mine genomic and metagenomic sequences and detect oleABCD biosynthetic gene clusters. To construct the pipeline an alignment of 68 OleC sequences in confirmed oleABCD gene clusters was asserribled (Sukovich et al. 2010) and a profile Hidden Markov Model (HMM) was built. Profile HMMs are probabilistic models used to detect remote sequence homologs (Durbin et al. 1998). A ‘profile’ is a consensus sequence, of a multiple sequence alignment which can be used to construct a position-specific scoring system for insertions, deletions, and substitutions. Profile HMMs are often more accurate, powerful, and sensitive than BLAST and other database search tools. The OleC Profile HMM was built using the open-source tool HMMR3 (Eddy 2011). The Profile HMM was used to query the UniProtKB database and extract the top 2500 hits (e-value<e⁻¹²¹). To visualize the taxonomic distribution of OleC homologs, a recently published “tree of life” was used as a template (Hug et al. 2016). There were OleC homologs in 608 different genera as displayed in the tree of life (OleC homologs are in red; FIG. 14). Interestingly, 6 genera with OleC homologs were detected in the Fungi and 1 in the Archaea in addition to 601 Bacterial genera spanning most major phyla in the tree of life with the exception of Candidate Phyla Radiation.

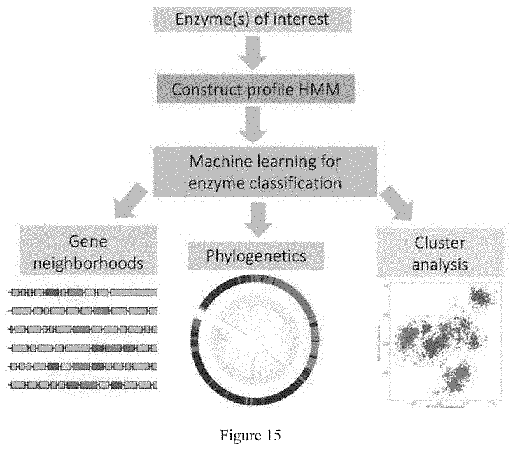

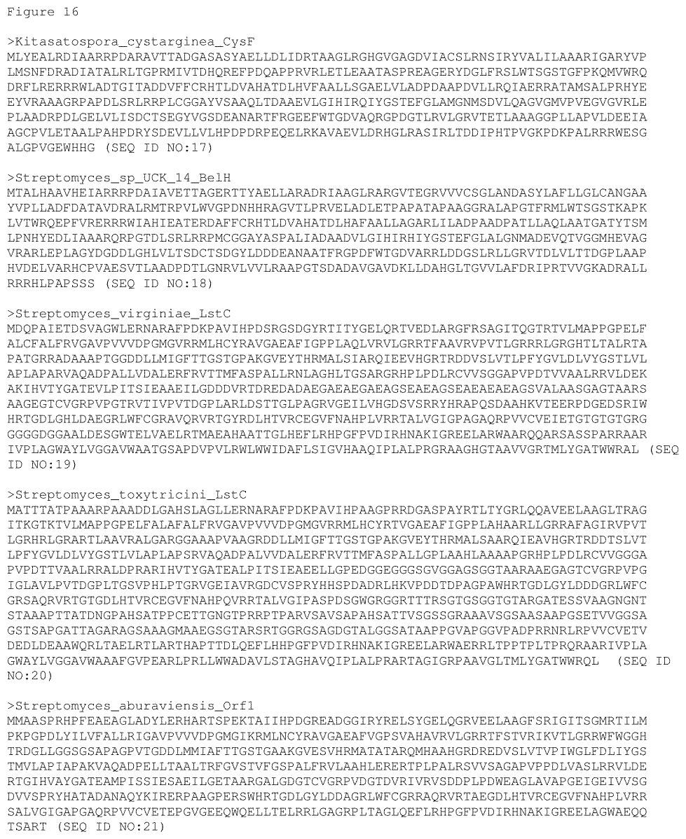

The purpose of the HMM search was to cast a wide net in order to encompass the potential diversity and abundance of species producing β-lactone compounds. However, homology does not necessarily imply similar enzyme function. In order to differentiate between ‘true’ OleC homologs and homologous enzymes likely catalyzing different reactions, we used a machine learning technique called Elastic Net. Elastic Net models use regularization to constrain the size of regression coefficients and perform variable selection to identify the most important features (e.g., amino acid residues) for classification (Lou & Elastic, 2005). Curated testing and training datasets for OleC and OleB sequences in the same enzyme superfamilies were assembled (see FIG. 15). The Elastic Net model was trained, tuned, and tested using the R package Unmet to differentiate ‘true Ole’ genes from non-Ole enzymes (Friedman et al., 2010). Following classification, 5 flanking genes on each side of the predicted ‘true OleC’ genes to identify their gene neighborhoods. The genomic context of ‘true OleB’ genes was used to predict whether the products of each biosynthetic gene cluster were likely to be olefins or-lactones. If an OleB homolog (β-lactone decarboxylase enzyme) was present in the gene neighborhood, we predicted the final product was likely to be olefin. If an OleB homolog was absent, it is predicted the final product would be a β-lactone natural product. This pipeline can be applied to mine genomic and metagenomics datasets for discovery of novel β-lactone biosynthetic gene clusters and natural products.

The disclosure also provides a computational method for identifying gene clusters encoding OleA, OleB, OleC and OleD enzymes in genomic and metagenomic DNA sequences or in gene repositories such as GenBank. In one embodiment, the disclosure provides a computational method known as “recommender systems”. The recommender system is trained with substrate specificity for known -lactone synthetase enzymes and their respective protein sequences. That is used to computationally predict substrate specificity by other lactone synthetase enzymes (there may be as many as 1000 sequences).

In another embodiment, the disclosure provides a homology model for the major broad-specificity β-lactone synthetase. This allows docking of potential substrates into the active site to determine the potential to make β-lactones.

### Other Embodiments

In one embodiment, a method to prepare β-lactones in vitro is provided one embodiment of the method one or more 3-hydroxy acid substrates and OleC or a homolog thereof but not OleB or OleD are combined under conditions that yield one or more oxetan-2-ones, in another embodiment of the method one or more acyl CoA substrates, one or more acyl substrate, one or more carboxylic acid substrates, or one or more fatty acid substrates and. OleA or a homolog thereof, OleC or a homolog thereof, and OleD or a homolog thereof but not OleB are combined under conditions that yield one or more oxetan-2-ones. In one embodiment, one or more 3-hydroxy acid substrates are combined with OleC or a homolo2 thereof, but not OleD or a homolog thereof or OleB or a homolog thereof that is enzymatically active in the decarboxylation of oxetan-2-ones. In one embodiment, one or more acyl CoA substrates, one or more carboxylic acid substrates, or one or more fatty acid substrates are combined with OleA or a homolog thereof, OleC or a homolog thereof and OleD or a homolog thereof but not OleB or a homolog thereof that is enzymatically active in the decarboxylation of oxetan-2-ones. In one embodiment, the one or more acyl CoA substrates are prepared by combining one or more carboxylic acids, CoA and a ligase. In one embodiment, the OleA or homolog thereof, the OleD or homolog thereof or the OleC or the homolog thereof or any combination thereof, are expressed in a heterologous cell. In one embodiment, the heterologous cell is a bacterial cell, a fungal cell, or a yeast cell. In one embodiment, the OleC or homolog thereof is isolated OleC or the homolog thereof, the OleA or homolog thereof is isolated OleA or the homolog thereof, or the OleD or the homolog thereof is isolated OleD or the homolog thereof. In one embodiment, the combining yields a plurality of distinct oxetan-2-ones. In one embodiment, the combining yields an oxetan-2-one. In one embodiment, the combining yields a plurality of distinct oxetan-2-ones and olefins. In one embodiment, the one or more oxetan-2-ones are isolated. In one embodiment, the oxetan-2-one has formula (I):

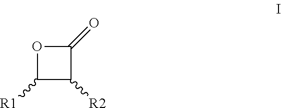

wherein each of R₁ and R₂ independently is a linear or branched alkyl, alkenyl, alkynyl, or aryl which is optionally substituted. In one emibodiment, the OleA or homolog thereof is combined with the one or more distinct ad CoAs before combining with the OleC or homolog thereof and the OleD or homolog thereof so as to increase the relative ratio of trans-β-lactones. In one embodiment, the OleA has at least 70%, 75%, 80%, 85%, 90%, 92%, 94%, 95%, e.g., 96%, 97%, 98% or 99%, amino acid sequence identity to a polypeptide encoded by SEQ ID NO:1; OleC has at least 70%, 75%, 80%, 85%, 90%, 92%, 94%, 95%, e.g., 96%, 97%, 98% or 99%, amino acid sequence identity to a polypeptide encoded by SEQ ID NO:3; or OleD has at least 70%, 75%, 80%, 85%, 90%, 92%, 94%, 95%, e.g., 96%, 97%, 98% or 99%, amino acid sequence identity to a polypeptide encoded by SEQ ID NO:4. In one embodiment, the OleA homolog comprises a polypeptide having at least 70%, 75%, 80%, 85%, 90%, 92%, 94%, 95%, e.g., 96%, 97%, 98% or 99%, amino acid sequence identity to SEQ ID NO:15; wherein the OleC homolog comprises a polypeptide having at least 70%, 75%, 80%, 85%, 90%, 92%, 94%, 95%, e.g., 96%, 97%, 98% or 99%, amino acid sequence identity to one of SEQ ID Nos. 17-21 or 25; or wherein the OleD homolog comprises a polypeptide having at least 70%, 75%, 80%, 85%, 90%, 92%, 94%, 95%, e.g., 96%, 97%, 98% or 99%, amino acid sequence identity to SEQ ID NO:16, 22, 23 or 24.In one embodiment, a LstA, LstB, LstD, NtlD, or NtlC is employed, e.g., one that comprises a polypeptide having at least 70%, 75%, 80%, 85%, 90%, 92%, 94%, 95%, e.g., 96%, 97%, 98% or 99%, amino acid sequence identity to one of SEQ ID Nos, 15, 26, 16, 22, 23 or 26.

In one embodiment, at least one of the OleA, the OleC and the OleD is from a different organism. In one embodiment, the method includes the use of an ATP regenerating system. In one embodiment, the OleA or homolog thereof, the OleC or homolog thereof and OleD or a homolog thereof are combined with fatty acids, CoA and a fatty acyl-CoA synthetase. In one embodiment, the OleA or homolog thereof, the OleC or homolog thereof and the OleD or homolog thereof are combined with decanoic-CoA and tetradecanoic-CoA.

Further provided is a method for increasing the ratio of trans lactones in a mixture of lactones, comprising: combining mixed diastereomers of an oxetan-2-one with OleB or a homolog thereof but not OleA or OleC, so as to yield a mixture with an increased amount of trans-β-lactones. In one embodiment, the OleB has at least 70%, 75%, 80%, 85%, 90%, 92%, 94%, 95%, e.g., 96%, 97%, 98% or 99%, amino acid sequence identity to a polypeptide encoded by SEQ ID NO:2 or a homolog thereof encoded by one of SEQ ID Nos. 13-14.

Also provided is a method to identify β-lactone synthetase activity, comprising: combining at room temperature and a pH of about 6 to about 8, a sample suspected of having β-lactone synthetase and a dialkene, a dialkyne or a compound with an alkene and alkyne group, so as to yield a mixture; and detecting in the mixture a change in UV absorbance over time, wherein a change in UV absorbance is indicative of the presence or amount of a β-lactone synthetase.

In one embodiment, a host cell is provided comprising a genome augmented with a nucleic acid encoding OleA or a homolog thereof, a nucleic acid encoding OleC or a homolog thereof and a nucleic acid encoding OleD or a homolog thereof, but which lacks OleB activity, wherein the host cell is heterologous to one or more of the OleA, or homolog thereof, the OleC or homolog therof, the OleD or the homolog thereof. In one embodiment, the host cell is a bacterial cell, a fungal cell or a yeast cell, in one embodiment, the nucleic acid encoding OleA or a homolog thereof, a nucleic acid encoding OleC or a homolog thereof, and a nucleic acid encoding OleD or a homolog thereof are linked. In one embodiment, the host cell has a mutated OleB gene. In one embodiment, at least one of the OleA, the OleC, or the OleD, is heterologous to the host cell. In one embodiment, the OleA is heterologous to the OleC or the OleD, the OleC is heterologous to the OleA or the OleD, the OleD is heterologous to the OleC or the OleA, the OleA is heterologous to the OleC and the OleD, the OleC is heterologous to the OleA and the OleD, or the OleD is heterologous to the OleC and the OleA. In one embodiment, the OleA has at least 70%, 75%, 80%, 85%, 90%, 92%, 94%, 95%, e.g., 96%, 97%, 98% or 99%, amino acid sequence identity to a polypeptide encoded by SEQ ID NO:1; OleC has at least 70%, 75%, 80%, 85%, 90%, 92%, 94%, 95%, e.g., 96%, 97%, 98% or 99%, amino acid sequence identity to a polypeptide encoded by SEQ ID NO:3; or OleD has at least 70%, 75%, 80%, 85%, 90%, 92%, 94%, 95%, e.g., 96%, 97%, 98% or 99%, amino acid sequence identity to a polypeptide encoded by SEQ ID NO:4. In one embodiment, the OleA homolog comprises a polypeptide having at least 70%, 75%, 80%, 85%, 90%, 92%, 94%, 95%, e.g., 96%, 97%, 98% or 99%, amino acid sequence identity to SEQ ID NO:15; wherein the OleC homolog comprises a polypeptide having at least 70%, 75%, 80%, 85%, 90%, 92%, 94%, 95%, e.g., 96%, 97%, 98% or 99%, amino acid sequence identity to one of SEQ Nos. 17-21; or wherein the OleD homolog comprises a polypeptide having at least 70%, 75%%, 80%, 85%, 90%, 92%, 94%, 95%, e.g., 96%, 97%, 98% or 99%, amino acid sequence identity to SEQ ID NO:16, 22, 23 or 24.

In one embodiment, a host cell comprising a genome expressing a heterologous OleC is provided. In one embodiment, the host cell is a bacterial cell, a fungal cell or a yeast cell. In one embodiment, the OleC has at least 70%, 75%, 80%, 85%, 90%, 92%, 94%, 95%, e.g., 96%, 97%, 98% or 99%, amino acid sequence identity to a polypeptide encoded by SEQ ID NO:3. The host cell may be employed with one or more 3-hydroxy acid substrates, one or more acyl substrates, one or more acyl CoA substrates, one or more distinct carboxylic acid substrates, or one or more distinct fatty acid substrates, so as to yield one or more oxetan-2-ones. in one embodiment, the one or more oxetan-2-ones are not expressed by a corresponding host cell that is not combined with the one or more substrates. In one embodiment, the substrates are exogenously added to the host cell.

### OleB Example

### Material and Methods

### Chemical Synthesis of β-Hydroxy Acids, β-Lactone, and Olefin.

All compounds, cis- and trans-β-octyl-4-nonyloxetan-2-one (β-lactones), 3-hydroxy-2-octyldodecanoic acid (13-hydroxy acids), cis- and trans-9-nonadecene (olefins) where chemically synthesized as described in Christenson et al.A (2017) Briefly, β-hydroxy acids were synthesized from decanoic acid and decanal and recrystallization yielded a 1:1:1:1 ratio of racemic diastereomers (Mulzer et al.,1981) The cis-β-lactone was synthesized from decanoic acid via a ketene dimer that was subsequently hydrogenated to yield a cis-β-lactone (Lee et al., 2005). Trans-β-lactone was separated from a cis- and trans-β-lactone mixture generated from the precursor β-hydroxy acid with sulfonyl chloride (Crossland et al., 1970). The cis-olefin was generated from the coupling of 1-decyne with 1-bromononane precursors followed by hydrogenation with Lindlar catalyst (Lindlar et al., 1952; Buck et al., 2001). Photoisomerization of the cis-olefin generated the trans-olefin standard (Thalmann et al., 1985).

Generating mutants of OleB and OleBC.

Site-directed mutations of OleB derived from the wild-type protein sequences from Xanthomonas campestris ATCC 33913 (WP_011437021.1) and Micrococcus luteus OleBC (WP_010078536.1) were made with New England Biolabs Q5 quick change site directed mutagenesis kit following manufacturer's instructions. All primers were ordered from Integrated DNA Technologies (IDT). To confirm each mutant, single colonies were grown in 5 mL, cultures at 37° C. overnight under kanamycin selection. Plasmids were isolated using a QIAGEN Miniprep kit and sent to ACGT Inc for sequencing.

Purification of OleB and OleBC fusion

The buffer for OleB purification contained 200 mM NaCl, 20 mM NaPO₄, 10% glycerol, and 0.5% PEG 400 (Hampton Research) at pH 7.4. E. coli BL-21 DE3 cells containing OleB with a 6× Histidine tag on the N-terminal were, grown, sonicated, and crude protein was purified using a Ni²⁺ column. Protein concentrations of purified OleB solutions were measured by Bradford assay. Purified OleB solutions were routinely stored at −80° C. OleBC and OleB_{D163A}C fusion proteins from Ml were generated with a 6× Histidine tag and purified as described previouslv.¹³

OleB Reactions with β-Lactone Followed by ¹H-NMR

For enzyme reactions, the appropriate substrate cis- or trans-β-lactone was first dissolved in ethanol at 0.17 mg/mL. Reactions were carried out in reparatory funnels containing 1.0 rug X. campestris OleB (or M. luteus OleBC fusion), 3.0 mL of the β-lactone substrate, 10 μl of 10% 1-bromonaphthalene as an internal standard, and 100 mL buffer (200 mM NaCl, 20 mM NaPO4, pH 7.4), and incubated at room temperature overnight. Reactions were extracted twice, with 10 ml and 5 ml methylene chloride, consecutively. The organic extracts were pooled and back-extracted with 15 ml double-distilled H₂O. The organic fraction was dried, dissolved in CDCl₃, and placed in 5 mm NMR tubes with tetramethylsilane (TMS) as a reference. A Varian Inova 400 MHz NMR spectrometer using a 5 mm Auto-X Dual Broadband probe at 20° C. was used for all spectral acquisitions. Spectra were typically acquired using 1,024 pulses with a 3 second pulse delay.

OleB Reactions with Haloalkanes

The following haloalkane substrates were dissolved in ethanol to a concentration of 5 mM for testing with Xc OleB: 1-iodobutane, 1,3-diiodobutane,1-chlorobutane, 1-bramopentane, 1-chlorohexane, 1-bromooctane, 1-iodoundecane, and 7-(bromomethyl)pentadecane. Reactions were carried out in glass GC vials contained purified OleB (40 μg) and 10 μL of substrate in 500 μL, of 200 mM NaCl, 20 mM NaPO₄ at pH 7.4. Reactions were incubated at room temperature overnight, followed by extraction with tert-butyl methylether (MTBE). The MTBE extract was transferred to a clean GC vial and analyzed by gas chromatography/mass spectrometry (GC/MS Agilent 7890a & 5975c with an Agilent J&W bd-ms1 column 30 m length, 0.25 mm diameter, 0.25 μm film).

### Bioinformatic Analysis of OleB and Haloalkane Dehalogenases

Sequences for representative merribers of the α/β-hydrolase protein superfamily were retrieved from the Protein Data Bank (PDB) using the SCOP classification for α/β-hydrolases and filtered to include only representative bacterial sequences for each protein family (FIG. 14). To obtain a higher resolution phylogeny for the relationship of OleB/OleBC sequences with haloalkane dehalogenases, accession numbers for characterized HLD-I, -II, and -III accession numbers were pulled from Nagata et al. (2015). Five experimentally characterized OleB and OleBC sequences were obtained from Sukovich et al. (2010). Protein sequences were aligned and curated using the DECIPHER package in R (Wright et al., 2015). Due to the length of OleBC fusion sequences interfering with proper alignment, the last 550 residues were trimmed to eliminate the OleC region. Maximum-likelihood phylogenies with 100 bootstrap replicates were inferred from alignment using the JTT method using the phangorn R package (scliep et al., 2011). A structural homology model for the Xanthomonas campestris OleB sequence (WP_012437021.1) was built using default parameters in Phyre2 (Kelley et al., 2015).

Mass Spectroscopy of Acyl-Enzyme Intermediate with Haloalkanes

To identify an acyl-enzyme intermediate, Matrix Assisted Laser Desorption Ionization (MALDI) was carried out on wild type OleB and OleB_{D114A} proteins that had been reacted with 7-(bromornethyl)pentadecane (TCI). These two substrates contain the reactive bromomethyl group in the middle of a long alkyl chain, thereby mimicking the β-lactone substrate of OleB. Reactions contained 500 μM substrate and 40 μg of OleB in 100 μL of buffer (20 mM NaCl, 5 mM NaPO₄, pH 7.4). Reaction were prepared for MALDI using standard C₄ ZipTip (Millipore) procedures and spotted on a plate with sinapinic acid. Samples were analyzed on a Bruker Autoflex Speed MALDI-TOF.

### Results

Purification of Monomeric OleB without Detergents.

The OleB protein from X. campestris had been purified previously in a study showing that OleB, OleC, and OleD combine to form large enzyme assemblies on the order of 2 MDa molecular weight (Christenson et al., 2017). The individual activity of the OleB protein was not demonstrated in that study. Moreover, in that previous report, OleB purification required the presence of 0.05% Triton X-100 to maintain the protein in a soluble form. Despite that, the purified OleB protein formed large, non-homogeneous aggregates when not in admixture with OleC and OleD. In the present study, it was discovered that the addition polyethylene glycol (PEG 400) to purification buffers stabilized OleB, making it more amenable to purification and concentration. Purification yields increased to 19 mg OleB/L of culture compared to 2 mg/L (Christenson et al., 2017). Unlike OleB purified in Triton X-100, the protein purified with PEG 400 migrated largely as a monomer as observed by gel filtration. This monomeric protein form was used in these studies, although the Triton-purified OleB was shown to catalyze the same reaction.

OleB Utilizes only Cis-β-Lactones

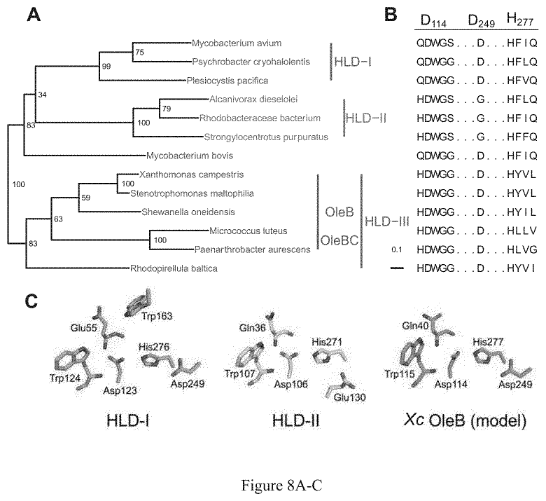

Previous studies had shown that OleA, OleD, and. OleC act sequentially to condense two fatty acyl-CoA molecules and produce a β-lactone ring with two C₉-C₁₄ chains appended. The final biologically-relevant product is a cis-olefin and indirect evidence was obtained previously that OleB might catalyze final step in the biosynthetic pathway. β-Lactones are known to undergo thermal decarboxylation to the corresponding olefins, but dialkyl β-lactones are stable at room temperature and neutral pH. To determine if OleB might catalyze a decarboxylation reaction, cis- and trans-β-octyl-4-nonyl-2-oxetanone (cis- and trans-β-lactone) were chemically synthesized. ¹H-NMR demonstrated that both the cis- and trans-β-lactones enantiomeric pairs contained <10% of the opposite configuration. ¹H-NMR analysis of OleB reactions showed that 47% of the cis-β-lactone underwent decarboxylation to the cis-olefin when allowed to react overnight to go to completion, indicating that OleB selectively acts on only one of the cis-β-lactone enantiomers (FIG. 7). An OleB reaction mixture with trans-β-lactone showed only 4% underwent decarboxylation to a cis-olefin. This 4% product is consistent with the small contamination of cis-β-lactone in the trans-β-lactone sample. Olefin was undetectable in control reactions lacking enzyme. A synthetic trans-olefin standard was prepared to aid in analytical methods, but there was no evidence for this compound in OleB reactions. This observation agrees with multiple literature reports that bacteria exclusively produce cis-olefins (Albro et al., 1969; Frias et al., 2009) To help determine if OleB has a preference for one cis-β-lactone enantiomer, reactions were run with double and quadruple the amount of OleB. Reactions with 2 mg and 4 mg of OleB converted 54% and 63% of the cis-β-lactone starting material to cis-olefin, suggesting that OleB preferentially acts on one of the cis-enantiomers. Taken together, these data supported the idea that OleB acts physiologically to catalyze decarboxylation of cis-β-lactones to yield cis-olefins that complete the olefin biosynthetic pathway.

OleB Clusters with Type-III Haloalkane Dehalogenases.

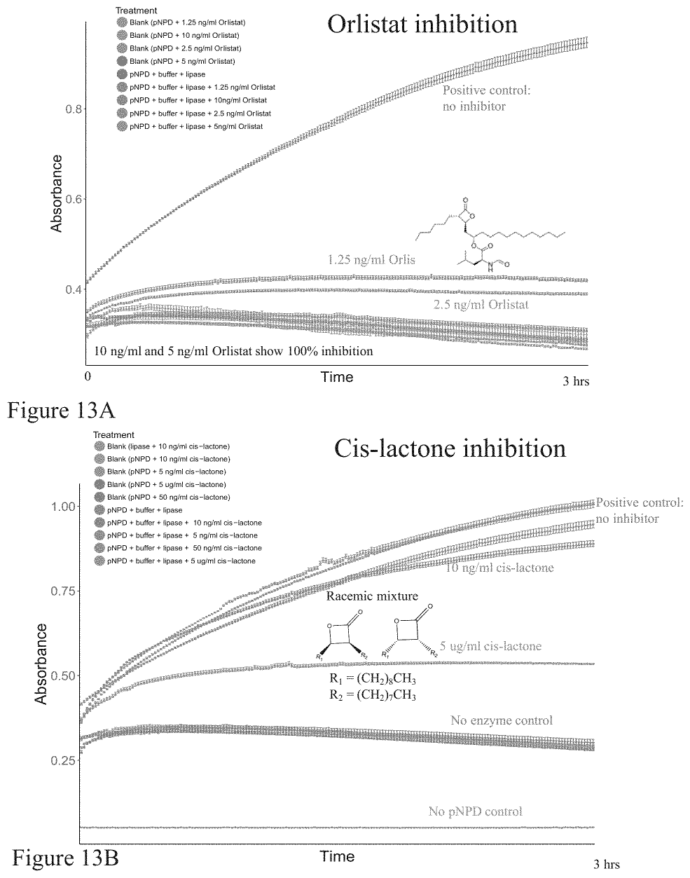

OleB had previously been demonstrated to be a member of the α/β-hydrolase superfamily (Sukovich et al., 2010b), but a deeper analysis of the nearest evolutionary relationships was not undertaken at that time. Here, a sequence was considered to be an OleB protein if it was derived from organisms shown to produce olefins or when the oleB gene homolog could be identified within 3 open reading frames of the oleACD genes. OleB protein sequences clustered most closely with haloalkane dehalogenases (HLDs) (FIG. 11). Within the OleB sequences, there were separate clusters for the OleB proteins found within most bacteria and the OleB domain of the OleBC fusion proteins found in Actinobacteria.

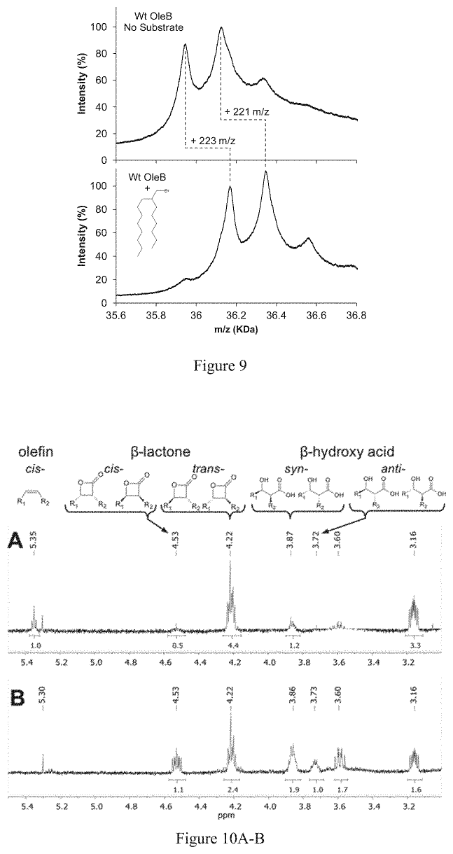

Because the α/β-hydrolase superfamily consists of highly divergent proteins, it was most insightful to conduct a phylogenetic analysis using only haloalkane dehalogenase (HLD) and OleB sequences. Phylogenetic analysis (FIG. 8) for a subset of HLDs and OleB proteins recovered the classification of HLDs into three subgroups: HLD-I, -II, and -III (Chovancova et al., 2007). Unexpectedly, OleB sequences were not a separate cluster, but were interspersed within the HLD-III subgroup. Closer analysis of the genomic regions for those 36 putative sequences suggested in Chovancova et al. (2007) found that 72% were part of an oleABCD gene cluster (FIG. 12), suggesting that those proteins function as β-lactone decarboxylases. Additionally, the only two HLD-III proteins characterized to date are reported to have “very low activities with typical substrates of haloalkane dehalogenases” (Jesenska et al., 2009). In light of this, at least a portion of the HLD of subgroup III may have been misannotated, and should instead be considered β-lactone decarboxylases.

### OleB and HLD Alignments and Site-Directed Mutagenesis Suggest Catalytic Residues

Sequence alignments of OleB proteins and well-studied HLDs were examined to identify residues that might be directly involved in catalysis. X-ray crystal structures and mutagenesis studies have delineated the catalytic residues and mechanistic features of class I and class II HLDs. By contrast, much less is known about class III HLD proteins and no structures are available.

Alignment of OleB from X. campestris with HLD suggested the presence of a catalytic triad in OleB represented by Asp₁₁₄, His₂₇₇ and Asp₂₄₉. These residues are completely conserved in all OleB sequences and align perfectly with HLD-I. The HLD-II enzymes are known to utilize a glutamate derived from the end of the β-sheet 6 in place of D249. Despite that, a comparison of X-ray structures from the HLD-I and -II classes with a homology model of the X. campestris OleB protein suggested that the catalytic triad of D114, D249, and H277 may be isostructural and isofunctional between OleB and all HLD proteins. The comparable D114 residue has been identified to serve as a nucleophile for halide displacement in HLD reactions. The backbone nitrogen of Trp124 and Glu55 from HLD-I and the equivalent Trp107 and Gln36 in HLD-II are known stabilize the oxyanion intermediate of haloalkane dehalogenation (Hesseler et al., 2011; Novak et al., 2014). The sidechain nitrogens of Trp124 and Trp163 in HLD-I and Trp107 and Gln26 in HLD-II are known to stabilize the displaced halide atom during the catalytic cycle(Hesseler et al., 2011; Novak. et al., 2014). The equivalent residues are completely conserved in OleB proteins.

Site-directed mutagenesis of the X. campestris OleB protein was conducted to test the hypothesis that the residues identified might comprise a catalytic triad. Three mutants were made and tested for activity: D114A, H277A, and D249A. OleB_{D114A} and OleB_{H277A} showed no detectable activity towards cis-β-lactones when monitored by ¹H-NMR. OleB_{D249A}, however, showed decarboxylation activity lower than wild-type OleB.

The ¹H-NMR assay used here did not allow us to measuring steady-state kinetic parameters. Moreover, the OleB substrates lack UV/Vis absorbance, have very poor solubility in water, and are thermally unstable making assays difficult. However, alternate assay methods are currently under investigation and may allow the determination of kinetic parameters in future studies.

### OleB Shows no Detectable Dehalogenase Activity Towards Haloalkane Substrates

OleB from Xc was tested with haloalkane substrates to assess potential dehalogenase activity. Jesenska et al. (2009) tested 30 haloalkane substrates with two purified HLD-III proteins and found very limited activity with a select nurriber of compounds. Substrates showing the highest activity in those studies and substrates containing long alkyl chains similar to native OleB substrates were tested here. No detectable activity was observed against the following haloalkane substrates: 1-iodobutane, 1-iodoundecane, 1-cholohexane, 1-bromohexane, 1-chlorobutane, 1-bromobutane, and 7-(bromomethyl)pentadecane when monitored by GC-FID/MS. The level of activity of activity for all haloalkane substrates was less than 0.2 h⁻¹ as no significant decrease in the halogenated substrate or appearance of alcohol or alcohol dehydration products was observed by GC-FID/MS.

### Haloalkane Substrate Mimic Forms Stable Acyl-Enzyme Intermediate

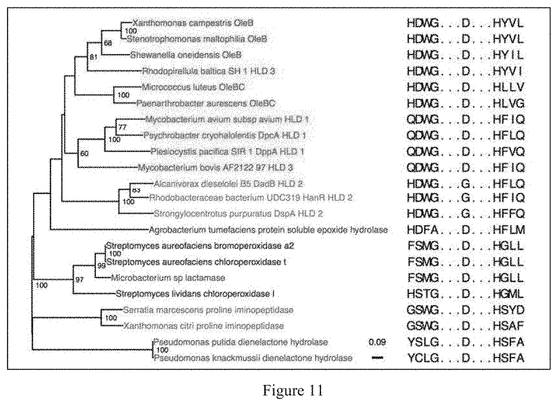

The reaction pathway of HID proteins are known to proceed through an acyl enzyme intermediate between the nucleophilic Asp and the substrate. To investigate whether OleB might form a covalent enzyme intermediate, OleB was reacted with 7-(hromomethyl)pentadecane and a shift in protein mass was examined by MALDI-TOF mass spectrometry. A mass shift in OleB corresponding to the mass of the debrominated alky chain was identified (FIG. 9). A parallel incubation without the brominated substrate analog served as a control and showed the expected mass of the wild-type OleB. Another experiment was conducted with the putative nucleophilic residue, D114, mutated to an unreactive residue alanine. The D114A mutant OleB enzyme did not show a mass shift when incubated with 7-(methylbromo)pentadecane, suggested of the role D144 is similarly to its function in haloalkane dehalogenases. The modified wild-type protein appeared to be stable, suggesting that if an acyl-intermediate is being formed, OleB is unable to remove the alkyl group.

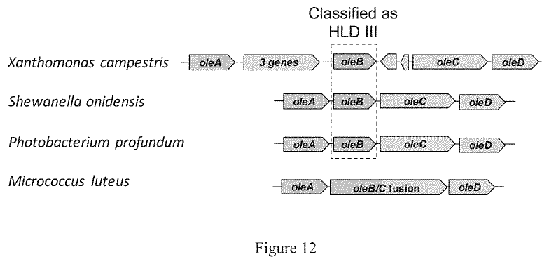

To ensure that previous findings are not confined to the single Xc OleB protein, the M. luteus OleBC fusion protein was purified and assayed here. The OleB domain of M. luteus is only 32% identical to Xc OleB and the OleC domain is known to have β-lactone synthetase activity (Christenson et al., 2017). OleC proteins are reported to accept all four β-hydroxy acids diastereomers, albeit at different rates, to generate all four possible β-lactone diastereomers (Christenson et al.; Kancharla et al., 2016). When the nucleophilic Asp of M. luteus OleBC fusion (Asp163) was imitated to Ala and reacted with OleC substrate, a mixture of all four syn- and anti-β-hydroxy acids, only trans- and cis-β-lactones were observed. However, under the same conditions, the wild-type Ml OleBC fusion protein formed less cis-β-lactone, and resonances consistent with cis-olefin appeared (FIG. 10). These findings are consistent with OleB acting on a single cis-β-lactone enantiomer to yield a cis-olefin in a reaction dependent on Asp163 as a nucleophile. Additionally, these data demonstrate that divergent sequences within the HLD-III subgroup have β-lactone decarboxylase activity.

### Discussion

OleB may be the first enzyme reported to decarboxylate a β-lactone to form a cis-olefin. There are other known α/β-hydrolase superfamily members from plants that perform decarboxylation reactions, such as MKS1 from Solarium habrochaites (wild tomato), that decarboxylate β-keto acids to methylketones (Auldridge et al., 2012). However, these show only about 12% sequence identity to Xc OleB and are reported to rely on a completely different mechanism. Additionally, α/β-hydrolase superfamily members, such as AidH, from Ochrobactrum sp. are known to hydrolyze five-membered γ-lactone rings of quorum sensing molecules to 4-hydroxy acids (Gao et al., 2013). Again, these lactonases show little sequence identity to OleB (-19%) and contain a serine at the catalytic nucleophile suggesting a different mechanism.

OleB appears to react preferentially with only one enantiomer of the synthetic cis-β-lactone pair. The preceding pathway enzymes, OleA and OleD, are known to generate the 2R,3S-configuration in the β-hydroxy acid. OleC is believed to retain this stereochemistry during its ring closure reaction to the β-lactone. As such, it is likely that OleB acts on the 2R,3S-β-lactone to produce a cis-olefin, but studies to identify the chirality of the remaining lactone must be conducted. No trans-olefin was ever observed, consistent with multiple literature reports that the Ole pathway exclusively produces cis-olefin (Albro et al. 1969; Frias et al., 2009).

Sequence alignments and homology modeling reveal OleB is closely related to HLDs. Chovoncova et al. described three subfamilies of HLDs (I, II, and III).¹⁴ However, 72% of the HLD-HI subfamily from this original work were found to be encoded in oleABCD gene clusters. Both subfamilies I and II have multiple crystal structures and the mechanisms of these enzymes are well understood, but no structures are available for HLD-IIIs. The two previously characterized subfamily III members have poor dehalogenase activity and the HLD-III OleB has no detectable dehalogenase activity (Jesenska et al., 2009). However, the annotated HLD IIIs, Xc OleB and Ml OleBC fusion, were found to have β-lactone decarboxylase activity, indicating at least part of this HLD-III subgroup is misannotated. Further bioinformatics work, coupled with biochemical data, is needed to distinguish between these two enzyme functions. Additionally, the enzymatic function of sequences that cluster with HLD-IIIs (OleBs), but are not part of oleABCD gene clusters, must be explored.

In both sequence and structural alignments, the conserved. Asp114 from Xc OleB aligns perfectly with the nucleophilic aspartic acid of haloallcane dehalogenases. Additionally, MALDI-MS of OleB and OleB_{D114A} implicates this Asp as the critical nucleophile to generate the canonical acyl enzyme intermediate in the HLD mechanism. The function of Xc OleB is dependent on His277 consistent with its complete conservation within both OleB and HLD sequences. The role of the second acidic residue (Asp249 in HLD Is or Glu130 in HLD-IIs) in maintaining the correct protonation state of His277 for the activation of water agrees with our data that Xc OleB is slower when Asp249 is mutated to an Ala. Considering the aforementioned data, we propose the following β-lactone decarboxylation mechanism for OleB.

### A) Known Haloalkane Dehalogenase Mechanism

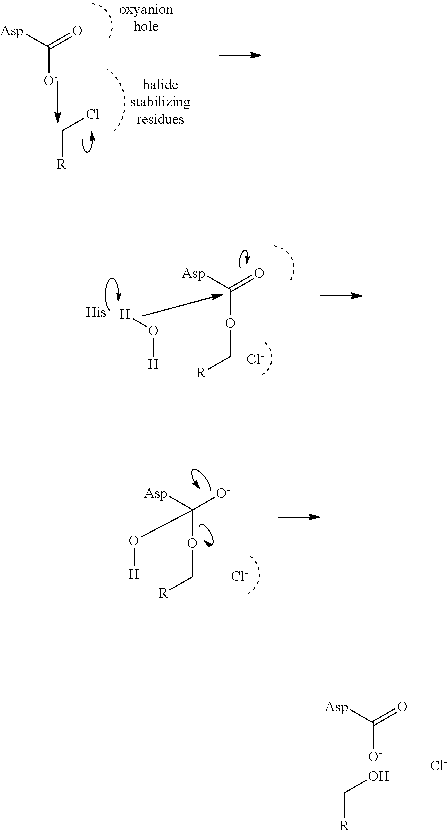

### B) Proposed OleB Mechanism

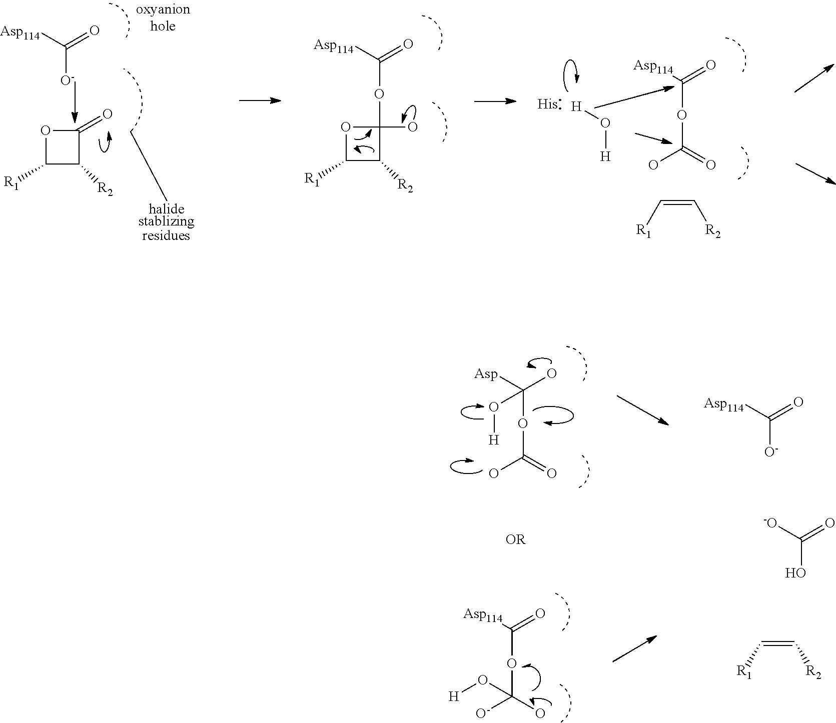

The canonical mechanism for HLDs and proposed mechanism for OleB are shown above. The nucleophilic Asp114 of OleB attacks the carbonyl carbon of the β-lactone ring to generate a tetrahedral intermediate. The side chains of Trp115 and Gln40 are in equivalent spatial and sequence positions to act as halide stabilizing residues, but no halide is present in the lactone moiety. Instead, these residues could act to stabilize the oxyanion in first tetrahedral intermediate. This first tetrahedral intermediate resolves to expel the olefin product and generate an anhydride as the equivalent to the acyl enzyme intermediate of HLDs.

There are now two possible centers for the attack of water activated by His277, the carbonyl of aspartic acid, or the carbonyl originating from the β-lactone. In favor of the Asp is the fact that this is the canonical pathway for HLDs and presumably contains the optimal bond angles and distances for attack. Additionally, the backbone nitrogens that create the oxyanion hole in HLDs (X of the H(XP motif and Trp adjacent to the nucleophile) are in the same spatial position in our model and are 100% conserved across all OleB sequences. However, in favor of the lower pathway is the biochemical evidence that no haloalkane substrates turn over with OleB. Hydroxide attack of the lower carbonyl nicely explains the trapping of the acyl-enzyme intermediate when OleB is reacted with 7(bromo-methyl)pentadecane. Additionally, the resulting second tetrahedral intermediate would be at the same site as the first proposed in step two. This mechanism is simpler, as OleB would only need to have the necessary residues to stabilize an oxyanion in one location rather than two. Regardless of the pathway, resulting products are identical: alkene, bicarbonate, and the regenerated enzyme.

In summary, OleB is concretely defined as the final step of the long-chain olefin biosynthesis pathway by decarboxylating the β-lactone product of OleC. OleB shows many similarities to haloalkane dehalogenases and comprises most of the sequences reported in the MX) subgroup III suggesting a misannotation of this group of enzymes. OleB proteins contain the conserved Asp-His-Asp/Glu catalytic triad of HLDs, and current evidence supports an analogous mechanism.

The invention will be described by the following non-limiting examples.

### EXAMPLE I

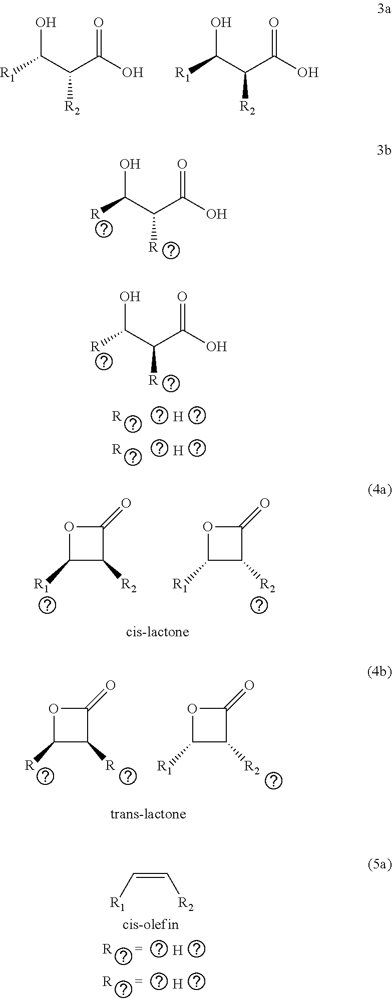

The first β-lactone synthetase enzyme is reported, creating an unexpected link between the biosynthesis of olefinic hydrocarbons and highly functionalized natural products. The enzyme OleC, involved in the microbial biosynthesis of long-chain olefinic hydrocarbons, reacts with syn- and anti-β-hydroxy acid substrates to yield cis- and trans-β-lactones, respectively. Protein sequence comparisons reveal that enzymes homologous to OleC are encoded in natural product gene clusters that generate β-lactone rings, suggesting a common mechanism of biosynthesis.

The β-lactone (2-oxetanone) substructure is well-known in organic synthesis and microbial natural products, some of which are presently being investigated for anti-obesity, anticancer, and antibiotic properties (Bai et al., 2014; Feling et al., 2003; Lee et al., 2005; Masamune et al., 1976). Although multiple organic synthesis routes exist for β-lactones (Wang et al., 2004), no specific enzyme that catalyzes the formation of this functional group had previously been identified. While defining the chemistry of a well-known olefinic hydrocarbon biosynthesis pathway, we identified a β-lactone synthetase whose presence extends into natural product biosynthesis.

The olefin biosynthesis pathway is encoded by a four-gene cluster, oleABCD, and is found in more than 250 divergent bacteria (Sukovich et al., 2010). Ole enzymes produce long-chain hydrocarbon cis-alkenes from activated fatty acids. OleA, the first enzyme of the pathway, has been studied in Xanthomonas campestris (Xc) and found to catalyze the head-to-head Claisen condensation of CoA-activated fatty acids (1) to unstable β-keto acids (2) (Frias et al., 2011). The second enzyme, OleD, couples the reduction of 2 with NADPH oxidation to yield stable β-hydroxy acids (3) as defined in Stentrophomonas maltophilia (Sm) (Bonnett et al., 2011). Finally, using gas chromatography (GC) detection methods, we have observed and others have reported that Sm OleC catalyzes an apparent decarboxylative dehydration reaction to generate the final cis-olefin product (Kancharla et al., 2016). Together, these findings left no defined purpose for the ever-present fourth gene in the cluster, oleB.

Using ¹H-NMR it was demonstrated that OleC proteins from four different bacteria produce thermally-labile β-lactones from β-hydroxy acids in an ATP-dependent reaction; no alkenes were observed. Further analyses of gene clusters for β-lactone-containing natural products reveal OleC homologs that likely perform this previously unknown biological β-lactone ring closure reaction.

The first suggestions of β-lactone synthetase activity arose when monitoring reactions of Xc OleC with ATP, MgCl₂, and a synthetic, diastereomeric mixture of 3 by GC. Two peaks were observed by GC, coupled to both a mass spectrometer and flame ionization detector (HD), with mass spectra and retention times identical to those of synthetic cis- and trans-olefin standards. However, the GC/FID peak areas of the enzymatically generated olefin varied significantly with GC inlet temperature and inlet liner purity, while synthetic standards were unaffected. This suggested that the observed olefin from OleC reactions may be thermal decomposition products of the actual OleC initial products.

To test this hypothesis, reactions of Xc OleC with 3 were scaled to generate sufficient quantities for ¹H-NMR. No resonances consistent with the prepared olefin standards were observed; rather, four distinct multiplets, each appearing as a doublet of doublets of doublets, arose. These resonances were consistent with the two hydrogens of cis- and trans-β-lactone rings and perfectly matched our authentic standards of cis- and trans-3-oetyl-4-nonyloxetan-2-one. Furthermore, when compounds 4a and 4b were analyzed by GC, retention times and mass spectra. matched those of olefin standards 5a and 5b, with sensitivity to inlet conditions being observed. The thermal decarboxylation of cis- and trans-β-lactone to cis- and trans-olefin, respectively, is well-known (Noyce et al., 1966; Mulzer et al., 1980). It is likely that thermal decomposition during GC/mass spectrometry (MS) analysis caused the product of OleC catalysis to be misidentified. Additionally, when supplemental NMR data from the literature report of Sm OleC characterization were reviewed, resonances of the cis- and trans-β-lactones, consistent with those described herein, are visible (Kancharla et al., 2016).

The stereochemical origins of 4a and 4b were then investigated by reacting Xc OleC with syn- and anti-β-hydroxy acids, 3. High-performance liquid chromatography was used to separate 3 into its syn- and anti-diastereomeric pairs (3a and 3b, respectively). Examining 3a and 3b by ¹H-NMR and GC/MS, post-methylation, demonstrated each contained <10% of the opposite racemic diastereomer. When reacting with Xc OleC, 3a produced 4a while 3b generated 4b. GC/MS analysis supported this conclusion, as OleC reactions with 3a and 3b yielded the β-lactone breakdown products, 5a and 5b, respectively. OleC consumed >90% of substrates 3a and 3b as determined by GC/MS, supporting the conclusions of Kancharla et al. that all four 33-hydroxy acid isomers are utilized by OleC (Kancharla et al., 2016). Taken together, Xc OleC represents the first reported β-lactone synthetase, converting β-hydroxy acid substrates to β-lactones in the presence of ATP and MgCl₂. Mg and ATP are likely required to activate the hydroxyl or carboxyl group and promote β-lactone ring formation.

To determine if β-lactone synthetase activity is a common enzymatic step in long-chain olefin biosynthesis, four oleC genes from oleABCD gene clusters in divergent microorganisms were obtained (Table 3). Purified OleC enzymes from the four organisms were reacted overnight with ATP, MgCl₂, and 3 and then analyzed by ¹H-NMR and GC/MS. The products of OleC proteins from the bacteria S. maltophilia, Arenimonas malthae, and Lysobacter dokdonensis were both 4a and 4b β-lactones, with no 5a or 5b olefins being observed, indicating that OleC enzymes from diverse sources are β-lactone synthetases. The Gram-positive bacterium Micrococcus luteus (Ml) was specifically chosen because its sequence diverges greatly from that of Xc OleC, and it contains a natural fusion of the oleB and oleC genes. This natural oleBC fusion is found in Actinobacteria, which comprise about 30% of the microorganisms that contain identifiable oleABCD genes. Reaction of the purified Ml OleBC fusion with MgCl₂, ATP, and 3 produced β-lactones 4a and 4b as well as small amounts of cis-olefin, 5a. No trace of trans-olefin, 5b, was detected. Further characterization is ongoing, but we believe that OleB performs a syn elimination of carbon dioxide from the cis-β-lactone to form the final cis-olefin product. This is consistent with previous studies of microorganisms expressing ole genes that contain olefins with a cis relative configuration exclusively (Sukovich et al., 2010; Albro et al., 1969; Frias et al., 2009). These data also demonstrate that an enzyme domain with an amino acid sequence only 35% identical to that of the Xc OleC generates β-lactones, indicating that this activity is likely common among all olefinic hydrocarbon biosynthesis OleC homologs.

TABLE 3
Other OleC Enzymes Make β-Lactones

| Organism | Accession no. | % ID^{a} |
|---|---|---|
| X. campestris | WP_011035474.1 | 100 |
| S. maltophilia | AFC01244.1 | 77 |
| A. malthae | WP_043804215.1 | 73 |
| L. dokdonensis | WP_036166093.1 | 70 |
| M. luteus^{b} | WP_010078536.1 | 35 |

^{a}Percent identity based on amino acid sequence.
^{b}OleC and OleB are a natural fusion in M. luteus.

Establishing the widespread nature of lactone synthetase activity within olefinic hydrocarbon biosynthesis led to the search of sequence databases for OleC homologs in other biosynthetic pathways. OleC is a member of the ubiquitous AMP-dependent ligase/synthetase enzyme superfamily; as such, homologs are found in all organisms (Conti et al., 1996). As of November 2016, a BLAST search of NCBI's non-redundant protein sequence database identified more than 900 sequences with >35% sequence identity and more than 16000 with >25% sequence identity to Xc OleC.

Of the sequences examined, two Xc OleC homologs were clearly encoded in gene clusters known to produce β-lactone natural products. The first, LstC, is an uncharacterized enzyme found in the lipstatin biosynthesis pathway from Streptomyces toxytricini. Lipstatin is the precursor to Orlistat, the only over-the-counter, Food and. Drug Administration-approved anti-obesity drug. LstC is a member of the AMP-dependent ligase/synthetase superfamily, and its protein sequence is 38% identical to that of Xc OleC, more similar than the sequence of the β-lactone synthetase domain of Ml OleBC (35%). Surprisingly, further investigation revealed homologs of OleA and OleD encoded by the lipstatin gene cluster, suggesting that the two gene clusters have a common ancestry. The syntheses of both lipstatin and olefinic hydrocarbons are initiated by the condensation of two fatty acyl-COAs to form a β-keto acid. In the case of lipstatin, the two fatty acids are 3-hydroxy-linoleic and octanoic acid Mai et al., 2014). The hydroxyl group of 3-hydroxy-linoleic acid is later functionalized by LstE and LstF with a modified valine (Bai et al., 2014). LstD and OleD likely perform the same NADPH-dependent reduction of the β-keto group to a hydroxyl group. Formation of the trans-β-lactone is likely accomplished by the OleC homolog LstC, to generate the final product in lipstatin biosynthesis. Olefin biosynthesis is completed by the putative OleB-dependent elimination of CO₂ to generate the final olefin product, The lipstatin gene cluster lacks any gene product that is homologous to OleB, consistent with the accumulation of the β-lactone natural product and further supporting our hypothesis that OleB performs the final step in the biosynthetic pathway to olefins,

The gene cluster responsible for the biosynthesis of ebelactone A, a commercially available esterase inhibitor, in Streptomyces aburaviensis shows a gene, odl, with an amino acid sequence 46% identical to that of Xc OleC and is directly adjacent to ebeA-G. Unlike lipstatin, ebelactone A is formed partly by a polyketide synthase multidomain protein rather than fatty acid condensation; as such, OleA and OleD homologs are not encoded in the surrounding gene cluster. Literature reports suggest that the β-lactone ring of ebelactone A is formed spontaneously from the final, enzyme-linked, β-hydroxy-thioester intermediate (Wyatt et al., 2013). While a spontaneous β-hydroxy-thioester cyclization is mechanistically plausible, β-lactone ftifmation from β-hydroxy-thioesters in ubiquitous pathways such as fatty acid oxidation or synthesis has not been reported to the best of our knowledge (Dick et al., 1996). Additionally, β-hydroxy-thioester intermediates are extremely common in polyketide synthesis pathways, while β-lactone formation is comparatively rare. An Orf1-independent cyclization would require a unique property of ebelactone A precursors or a novel polyketide domain architecture to promote β-lactone ring cyclization. However, in favor of an Orf1-independent mechanism is the fact that no thioesterase domain exists in the final polyketide synthesis domain, suggesting that no free β-hydroxy acid is released for the putative ATP-dependent Orf1 to act on. Other polyketide-type β-lactone gene clusters, such as those for salinosporamide A, cinnabaramide, and oxazolomycin, do not encode an OleC homolog with high sequence identity (>35%) in the vicinity of the cluster. Polyketide-derived β-lactones are thought to form by the cyclization of the final thioester, enzyme-linked intermediate, but this has never been characterized (Feling et al., 2003; Rachid et al., 2011; Zhao et al., 2010; Hemmerling et al., 2016). It is reasonable to hypothesize that specialized polyketide synthase domains represent a second mechanism of β-lactone formation. Regardless, the discovery of a stand-alone β-lactone synthetase here creates new opportunities for the natural product field. Preliminary screening of Streptomyces and Nocardia genomes suggests that β-lactone natural products may be more widespread than currently realized.

### EXAMPLE II

Bacterial β-lactone natural products have demonstrated anti-tumor, anti obesity, and anti-microbial properties. The oleC gene, encoding β-lactone synthetase, was frequently detected in biosynthetic gene clusters (BGCs) adjacent to oleB. The OleB protein is an unusual α/β-hydrolase superfamily member catalyzing decarboxylation of β-lactones to generate olefins. Bacteria possessing oleC but lacking oleB may secrete p-lactone natural products. Indeed, two Streptomycesstrains containing oleC homologs but lacking oleB were shown to produce the clinically-relevant β-lactone compounds lipstatin and ebelactone. A. Based on these results, a bioinformatics pipeline was developed to predict likely compounds produced by bacterial BGCs encoding β-lactone synthetases. The predictive framework detects ole BGCs in bacterial genomes and uses supervised learning to classify the predicted natural products as β-lactone compounds or olefins. The predictive framework was used to identify Streptomyces and Nocardia strains likely producing β-lactone natural products.

### EXAMPLE III

The following combinations of side groups R₁ and R₂ were used to prepare β-lactones structures either in vitro or in bacteria which were subjected to gas chromatography. Reaction conditions are typically 50 μM acyl-CoA substrate, 10 ug of each appropriate enzyme, buffer (200 mM NaCl, 20 mM4 NaPO₄ pH 7.4).

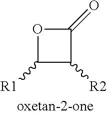

| R1 | R2 |
|---|---|
| 2-hydroxy-4,7-dodecadienyl | hexyl |
| pentadeca-3,6,9,12-tetraenyl | tetradec-all cis-2,5,8,11-tetraenyl |
| 10-pentadecenyl | 9-tetradecenyl |
| 14-methylpentadecyl | 13-methyltetradecyl |
| 13-methylpentadecyl | 12-methyltetradecyl |
| heptyl | octyl |
| nonyl | octyl |
| undecyl | decyl |
| tridecyl | dodecyl |
| pentadecyl | tetradecyl |
| 10-pentadecenyl | decyl |
| 10-pentadecenyl | dodecyl |
| undecyl | 9-tetradecenyl |
| tridecyl | 9-tetradecenyl |
| tridecyl | decyl |
| undecyl | dodecyl |

More compounds have been observed in vivo that have varying degrees of unsaturation (mono-,di-, tri-unsaturated bonds) with various alkyl chain lengths and branching. Other compounds are prepared using an alkane with an aryl (benzene group) attached and heterocyclic ring structures like imidazole. Functional groups that may be included in R1 or R2 include but are not limited to hydroxyl, halide, cyano, nitro, ketone, and amino groups. The length of the carbon chain may be up to about 8, 9, 10, 11, 12, 13, 14, 15, 16, 17, 18, 19 or 20 carbons.

### EXAMPLE IV

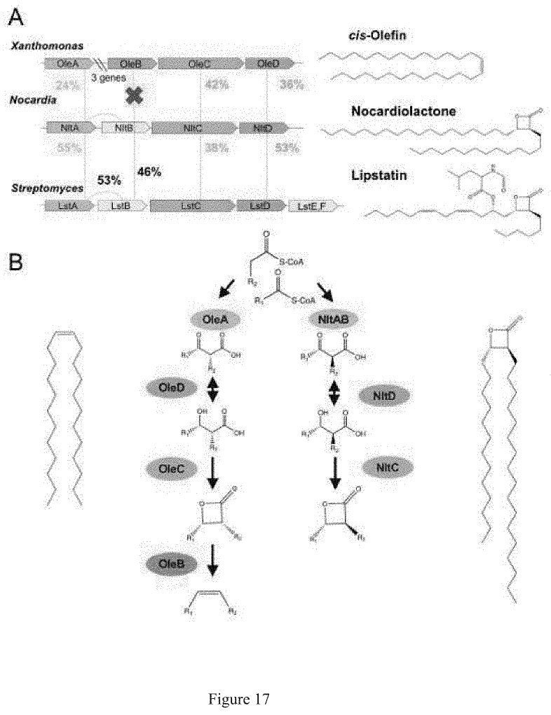

There is a pathway in Nocardia brasiliensis and over 70 other Nocardia spp, that produces a trans-β-lactone natural product, nocardiolactone (Mikami et al., 1999). The nocardiolactone gene cluster, nltABCD, was identified based on homology to the oleABCD biosynthetic pathway with the exception of NltB, which is not a homolog of OleB (FIG. 17A). Since there is no β-lactone decarboxylase (OleB), the final product contains a β-lactone moiety. Whereas the olefin biosynthetic pathway produces (2R,3S)-cis-β-lactones as intermediates to cis-olefins, structural elucidation indicates nocardiolactone contains a (2S, 3S)-trans-β-lactone moiety.

Trans-β-lactones have been shown to have stronger antibiotic properties than cis-β-lactones, therefore, controlling the stereochemistry has a direct effect on bioactivity. As one example, a previous study that synthesized four chiral β-lactone isomers of hymeglusin (DU-6622), found that different trans-β-lactone stereoisomers inhibited pancreatic lipase and/or HMG-CoA synthase in the micromolar IC₅₀ range whereas the cis-analogs had poor inhibitory activity (Tomoda et al., 1999). Due to the higher potency of trans-β-lactones as pharmacophores, enzymatic methods to produce trans-β-lactone moieties are of interest. Enzymes from Nocardia were heterologously expressed and combined in ‘one pot’ in vitro mixtures with olefin biosynthetic enzymes to produce exclusively cis- or trans-β-lactones. These results represent the first example of stereospecific control of β-lactone biosynthesis in vitro and lay the foundation towards engineering stereoselective β-lactone pathways in heterologous hosts.

Genetic manipulation in Nocardia is challenging due to the lack of well-established protocols. Therefore, the role of each enzyme in the pathway in vitro was identified through heterologous expression in E. coli BL21 and protein purification followed by enzyme activity assays. This approach gave full control over the pathway steps through direct chemical analysis of intermediates and comparison to authentic standards. It was found that a complete pathway to a di-alkyl-substituted trans-β-lactone could be reconstituted in vitro, and furthermore that we could mix-and-match with enzymes from nocardiolactone and the olefin biosynthetic pathways. For example, the unstable NltAB complex was substituted out from N. brasiliensis with the functionally-equivalent and stable homodimer, OleA, from X. campestris to catalyze the Claisen condensation of two acyl-CoAs to form 2-alkyl-3-ketoalkanoic acid. The pathway was then completed through OleD- or NltD-catalyzed reduction to 2-alkyl-3-hydroxyalkanoic acid followed by OleC- or NltC-catalyzed β-lactone formation

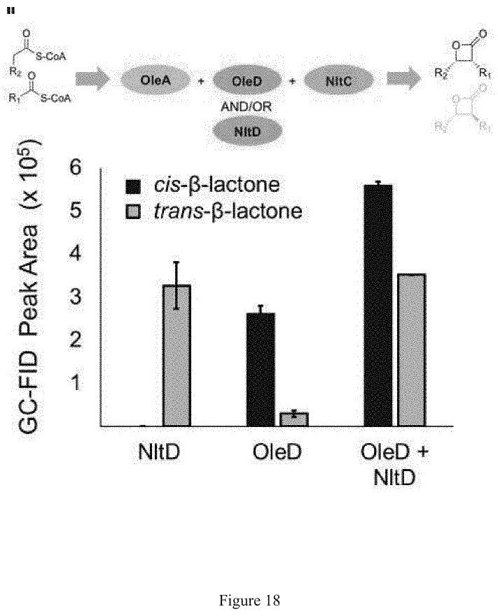

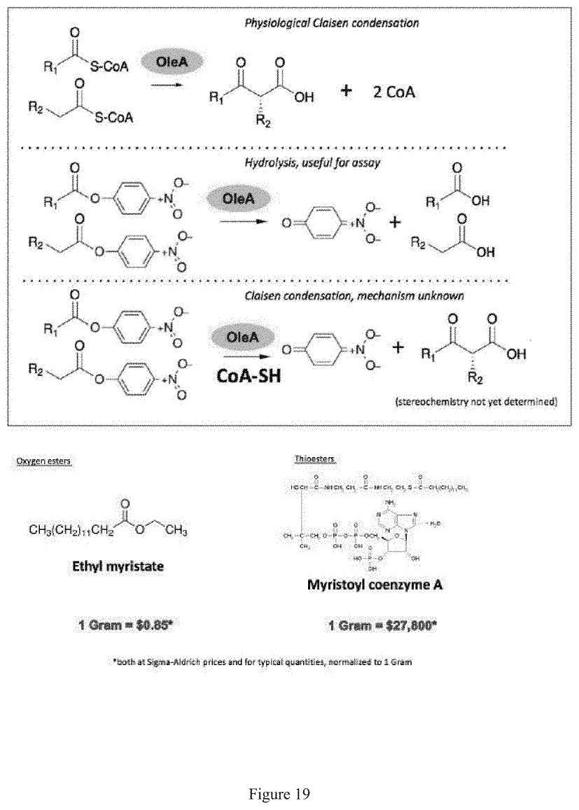

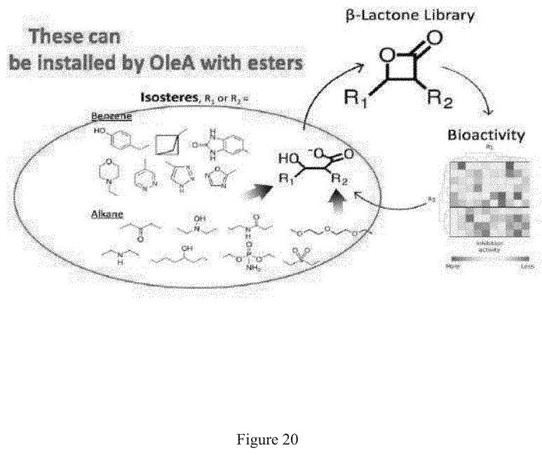

It was observed that the reductase enzymes in the pathway (OleD/NltD) determined the stereochemistry of the final β-lactone product. To test this, a ‘one-pot’ enzymatic synthetic scheme was used to achieve different β-lactone configurations through combinatorial mixtures of OleA, OleD, and OleC from the olefin pathway with NltC and NltD from nocardiolactone pathway. The addition of either NltD or OleD was sufficient to control stereochemistry of the final product (FIG. 18). NltC and OleC did not appear to exert stereospecific control, e.g., both cis- and trans-β-lactones were produced depending on the configuration of the hydroxy acid precursor. The combination of OleA+NltD+OleC yielded 100% trans-β-lactone, while OleA+OleD+OleC yielded an approximate 9:1 ratio of cis:trans (90% cis, 10% trans). The addition of equimolar amounts of OleA+NltD+OleD+OleC resulted in an approximate 1.6:1 mixture of cis- and trans β-lactone products (61% cis, 39% trans).

These results can be extended to enzymes in other pathways that likely are also involved in production of trans-β-lactone moieties in lipstatin- and esterastin-like pathways (SEQ ID Nos. 22-24). SEQ ID Nos. 22-24 all have less than 70% identity to SEQ ID NO: 16. Exemplary homologs of OleD/LstD/NltD that could be used to produce trans-β-lactone moieties in combination with OleA+OleC are as follows:

>WP_042260949.1 NAD-dependent epirnerase/
dehydratase family protein (NtlD)[Nocardia
brasiliensis]
(SEQ ID NO: 22)
MSKVLVTGASGFLGGALVRRLIRDGAHDVSILVRRTSNLADLGPDVDKVE
LVYGDLTDAASLVQATSGVDIVFHSAARVDERGTREQFWQENVRATELLL
DAARRGGASAFVFISSRSALMDYDGGDQLDIDESVPYPRRYLNLYSETKA
AAERAVLAADTTGFRTCALRPRAIWGAGDRSGPIVRLLGRTGTGKLPDIS
FGRDVYASLCHVDNIVDACVKAAANPATVGGKAYFIADAEKTNVWEFLGA
VATRLGYEPPSRKPNPKVIDAVVGVIETIWRIPAVATRWSPPLSRYAVAL
MTRSATYDTGAAARDFGYQPVVDRETGLATFLAWLEKQGGAVELTRTLR
>WP_068691876.1 NAD-dependent epimerase/
dehydratase family protein (NtlD) [
[Thermobifida halotolerans]
(SEQ ID NO: 23)
MRVLVTGASGFLGSHVAEACLRAGDEVRALVRPTSDPGHLRTLPGVEIVH
DLGDTASLRAAAEGVDVVHHSAARVLDHGSRAQFWDTNVEGTRRLLEAAR
DGGARRFVFVSSPSAVMDGRDQVDVDESIPYPRRYLNLYSQTKAAAERLV
LAADAPGFTTCALRPRAVWGPRDRHGFMPKLLGRLLAGRLPDLSGGRRVT
AALCHCANAAHACVLAARADGVGGRAYFVTDAEPVDVWAFMAEVAEMFG
APPPRRRVPPVLRDALVEAVELAWRMPFLAHHHDPPLSRYSVALLTRSST
YDTAAARRDLGYRPLVDRSTGLEGLRSWVEEIGGPGVWTEGAR
>WP_130512602.1 NAD-dependent epimerase/
dehydratase family protein [Krasilnikovia
cinnamomea] (NtlD) [
(SEQ ID NO: 24)
MKILVTGASGFLGGHIAEAAVAADHDVRALLRPTAALSMDAGADRVEPVR
GDLTDPASLAVATAGVDVVIHSAARVTDHGSPAQFHDTNVAGTQRLLAAA
RANGVSRFVFVSSPSAVMDGTDQVGIDESTPYPAKYLNLYSETKAAAERL
VLAANEPGFTTSALRPRGIWGPRDWHGFMPRLIAKLRAGRLPDLSGGRTV
LASLCHATNAAHACLLAAGSDRVGGRAYFVADAEVSDVWALIAEVGAMFG
AAPPTRRVPPAVRDALVATIETVWRVPYLRDRYSPPLSRYSVALLTRSST
YDTSAAARDFGYAPLLDQPTGLRQLREWVDGIGGVDAFTRYVR

The OleC homolog from the nocardiolactone pathway in N. brasiliensis (having 42% amino acid identity to X. campestris OleC) is an active β-lactone synthetase. An exemplary homolog of OleC includes polypeptides having at least 70%, 75%, 80%, 85%, 90%, 92%, 94%, 95%, e.g., 96%, 97%, 98% or 99%, amino acid sequence identity to SEQ ID NO:25.

>WP_042260945.1 AMP-binding protein(NtlC) [
[Nocardia brasiliensis]
(SEQ ID NO: 27)
MSSATYWQAIDRFRAFARAEPDREAVIYPVGTDAAGLPAYRHISYRELDD
WSETIAERLTASGVGSGTRT
IVLVLPSPELYAILFALLKIGAVPVVIDPGMGLRKMVHCLRAVEAEAFIG
IPPAHAVRVLFRRSFRKVRT
TVTVGKRWFWRGAKLAAWGTTPSGGAVDRVPADPGDVLVIGFTTGSTGPA
KAVELTHGNLASMIDQVHTA
RGEIAPETSLITLPLVGILDLLLGSRCVLPPLIPSKVGSTDPAHVAHAIE
TFGVRTMFASPALLIPLLRH
LEQQPNELKTLASIYSGGAPVPDWCIAGLRAALTDDVQIFAGYGSTEALP
MSLIESRELFDGLVERTHRG
EGTCIGRPADRIDARIVAITDDPIPTWARAEELAGDLARSRGIGELVVAG
PNVSTHYYWPDTANRQGKIV
DGDRIWHRTGDLAWIDDAGRIWFCGRKSQRVVTADGPMFTVQVEQIFNTV
AGVARTALVGVGAPGAQRPV
LCIELKPDAEGAAVGAALRARGAEFDLSRPIADFLIHPGFPVDIRHNAKI
GREQLAQWAGEQLGARA

### EXAMPLE V

OleA, a member of the thiolase superfamily, is known to catalyze the Claisen condensation of long-chain acyl-CoA substrates, initiating metabolic pathways in bacteria for the production of membrane lipids and β-lactone natural products. Bioinforrnatic methods and a high-throughput assay, in vivo and in vitro, were used to identify, purify and characterize bacterial OleA enzymes. The assay/screen is based on the discovery that OleA displayed surprisingly high rates of p-nitrophenyl ester hydrolysis. The high rates allowed activity to be determined with 1 ug protein in vitro and with heterologously expressed OleA in vivo. In addition,w it was found that p-nitrophenyl esters can substitute for CoA esters to make the physiological β-keto acid product when coenzyme A is provided. The coenzyme A is not consumed in the reaction and can be recycled. This is significant commercially because many p-nitrophenyl esters sell for $10 per gram whereas a typical CoA ester sells for $10,000 per gram. Moreover, a very large number of p-nitrophenyl esters can be synthesized from inexpensive fatty acids with one very simple chemical synthetic step. This advancement allows for the transformation of inexpensive fatty acid esters to β-lactones using a combination of OleA, OleD, OleC and recycling CoA.

### EXAMPLE VI

OleC enzymes can be reacted with β-hydroxy acid substrates or multiplexed with OleA, OleD and activated acyl precursors to make β-lactone libraries through one-pot enzymatic synthesis. Activity of the X. campestris β-lactone synthetase has been demonstrated with more than a dozen different β-hydroxy acid precursors with C6-C15 alkyl-, hydroxyallcyl-, alkenyl, alkynyl-, and phenyl-tails. The native pathway β-lactone product has a (2R,3S) configuration, but OleC still reacts to completion with both syn- and anti-β-hydroxy acids to make cis- and trans-β-lactones, respectively. OleA and OleD homologs prefer different chain length, branching and stereoconfiguration. Through mixing and matching enzymes with different substrate preferences, diverse combinations of syn- andlor anti-β-hydroxy acid diastereomers can be prepared to produce desired cis- and/or trans-β-lactone libraries.

β-Lactone libraries produced through (chemo)enzymatic methods can be screened for inhibition of desired or unique oxidoreductase, ligase, transferase, or hydrolase targets. Note that a major limitation here is the availability and expense of the substrates typically activated by CoA. In this context, acyl-transfer to the active cysteine in OleA using activated esters other than acyl-thioesters may be employed.

### REFERENCES

Albro et al., Biochemistry, 8:394 (1969).
Auldridge et al., Plant Cell., 24:1596 (2012).
Bai et al., Appl. Environ. Microbiol., 80:7473 (2014).
Beller et al., Appl, Environ. Microbiol., 76:1212 (2010).
Blom et al., Cold Spring Harb. Perspect. Biol. 3:a004713 (2011).
Boehringer et al., J. Mol. Biol., 425:841(2013).
Bonnett et al., Biochemistry, 50:9633 (2011).
Bradford, Anal Biochem., 72:248 (1976).
Buck and Chong, Tetrahedron Lett., 42:5825 (2001).
Channon and Chibnall Biochem. J., 23:168 (1929).
Chovancova et al., Proteins Struct. Funct. Genet., 67:305 (2007).
Christenson et al., Biochemistry., 56:348 (2017).
Christenson et al., J. Bacteriol., 199 (2017).
Conti et al., Structure, 4:287 (1996).
Crossland and Servis, J. Org. Chem., 35:3195 (1970).
Dick et al., J. Biol. Chem., 271:7273 (1996).
Durbin et al., Cambridge University Press (1998).
Eddy, PLoS Comput. Biol., 7:e1002195 (2011).
Enderle and McCarthy, Acta. Crystallogr. F. Struct. Biol. Commun., 71:1401 (2015).
Feling et al., Angew. Chem., Int. Ed., 42:355 (2003).
Frias et al., Appl. Environ. Microbial., 75:1774 (2009).
Frias et al., J. Biol. Chem., 286:10930 (2011).
Friedman and DaCosta, International patent WO/2008/147781 (2008).
Friedman et al., J. Stat. Softw., 33:1 (2010).
Gao et al., Acta. Crystallogr. Sect. D. Biol. Crystallogr., 69:82 (2013).
Goblirsch et al., Biochemistry. 51:4138 (2012).
Goblirsch et al., J. Biol. Chem., 291:26698 (2016).
Haase et al., Methods Mol. Biol., 1146:15 (1981).
Hemmerling and Hahn, J. Org. Chem., 12:1512 (2016).
Hesseler et al., Appl. Microbial. Biotechnol., 91:1049 (2011).
Hug et al., Nature Microb., 1:6 (2016).
Jesenská et al., Appl. Environ. Microbial., 75:5157 (2009).
Kancharla et al., Chem. Bio. Chem., 17:1426 (2016).
Kelley et al., Nat. Protoc., 10:845 (2015).
Koudelakova et al., Biochem. J., 435:345 (2011).
Ladenstein et al., FEBS J., 280:2537 (2013).
Lee et al., J. Am. Oil Chem. Soc., 82:181 (2005).
Lenfant et al., Nucleic Acids Res., 41:D423 (2013).
Lindlar, Helv. Chim. Acta., 35:446 (1952).
Masamune et al., J. Am. Chem. Soc., 98:7874 (1976).
Mikarni et al., Natural Products Letters, 13:277 (1999).
Mulzer et al., Angew. Chem., 92:469 (1980).
Mulzer et al., Chem. Ber., 114:3701 (1981).
Nadano et al., Biochemistry, 40:15184
Nagata et al., Appl. Microbiol. Biotechnol., 99:9865 (2015).
Nardini and Dijkstra, Curr. Opin. Struct. Biol., 9:732 (1999).
Nichols et al., FEMS Microbial. Lett., 125:281 (1995).
Novak et al., FEBS Lett., 588:1616 (2014).
Noyce and Bailin, J. Org. Chem., 31:4043.
Pawar et al., J. Biol. Chem., 256:3894 (1981).
Rachid et al, Chem. Bio. Chem., 12:922 (2011),
Rauwerdink and Kazlauskas, ACS Catal., 5:6153 (2015).
Robbins et al., Curr. Opin. Struct. Biol., 41:10 (2016).
Robinson et al., Nat. Prod. Reports, 36:458 (2019).
Robinson et al., Chembiochem., Mar. 11 (2019).
Schliep, Bioinformatics, 27:592 (2011).
Shevchenko et al., Anal Chem., 68:850 (1996).
Smith et al., Curr. Opin. Struct. Biol., 31:9 (2015),
Sukovich et al., Appl. Environ. Microbiol., 76:3850 (2010).

Sukovich et al., Journal of Bacteriology, 199:9 (2017).
Thalmann et al., Org. Synth., 63:192 (1985).
Tomoda et al., Biochemical and Biophysical Research Communications, 265:536 (1999).
van Loo et al., Appl. Environ. Microb., 72:2905 (2006).
Wang et al., Heterocycles, 64:605 (2004).
Wright, BMC Bioinformatics, 16:322 (2015).
Wyatt et al., J. Antibiot., 66:421 (2013).
Zhao et al., Curr. Opin. Biotechnol., 14:583 (2003).
Zhao et al., J. Biol. Chem., 285:20097 (2010).
Zou et al., J. Royal Stat. Soc. Series B-Stat. Method, 67:301 (2005).

All publications, patents and patent applications are incorporated herein by reference. While in the ftifegoing specification, this invention has been described in relation to certain preferred embodiments thereof, and many details have been set forth for purposes of illustration, it will be apparent to those skilled in the art that the invention is susceptible to additional embodiments and that certain of the details herein may be varied considerably without departing from the basic principles of the invention.

1. A method to prepare β-lactones in vitro, comprising:
combining one or more acyl CoA substrates, one or more activated acyl substrates, one or more carboxylic acid substrates, or one or more fatty acid substrates with OleA or a homolog thereof, OleC or a homolog thereof, and OleD or a homolog thereof but not OleB, under conditions that yield one or more oxetan-2-ones.

2. The method of claim 1 wherein one or more 3-hydroxy acid substrates are combined with GleC or a homolog thereof, but not OleD or a homolog thereof or OleB or a homolog thereof that is enzymatically active in the decarboxylation of oxetan-2-ones.

3. The method of claim 1 wherein one or more acyl CoA substrates, one or more carboxylic acid substrates, or one or more fatty acid substrates are combined with OleA or a homolog thereof, OleC or a homolog thereof and OleD or a homolog thereof but not OleB or a homolog thereof that is enzymatically active in the decarboxylation of oxetan-2-ones.

4. The method of claim 1 wherein the one or more acyl CoA substrates are prepared by combining one or more carboxylic acids, CoA and a ligase.

5. The method of claim 1 wherein the OleA or homolog thereof, the OleD or homolog, thereof or the OleC or the homolog thereof or any combination thereof, are expressed in a heterologous cell.

6. The method of claim 5 wherein the heterologous cell is a bacterial cell, a fungal cell, or a yeast cell.

7. The method of claim 1 wherein the OleC or homolog thereof is isolated OleC or the homolog thereof, the OleA or homolog thereof is isolated OleA or the homolog thereof, or the OleD or the homolog thereof is isolated OleD or the homolog thereof.

8. The method of claim 1 wherein the combining yields a plurality of distinct oxetan-2-ones, an oxetan-2-one or a plurality of distinct oxetan-2-ones and olefins.

9. The method of claim 1 wherein the oxetan-2-one has formula (I): 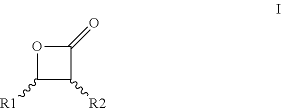
wherein each of R₁ and R independently is a linear or branched alkyl, alkenyl, alkynyl, or aryl which is optionally substituted.

10. The method of claim 1 wherein the OleA or homolog thereof is combined with the one or more distinct acyl CoAs or one or more distinct activated acyl substrates before combining with the OleC or homolog thereof and the OleD or homolog thereof so as to increase the relative ratio of trans-β-lactones.

11. The method of claim 1 wherein the OleA has at least 70%, 75%, 80%, 90%, 92%, 94%, 95%, e.g., 96%, 97%, 98% or 99%, amino acid sequence identity to a polypeptide encoded by SEQ ID NO:1; OleC has at least 70%, 75%, 80%, 85%, 90%, 92%, 94%, 95%, e.g., 96%, 97%, 98% or 99%, amino acid sequence identity to a polypeptide encoded by SEQ NO:3; or OleD has at least 70%, 75%, 80%, 85%, 90%, 92%, 94%, 95%, e.g., 96%, 97%, 98% or 99%, amino acid sequence identity to a polypeptide encoded by SEQ ID NO:4 or wherein the OleA homolog comprises a polypeptide having at least 70%, 75%, 80%, 85%, 90%, 92%, 94%, 95%, e.g., 96%, 97%, 98% or 99%, amino acid sequence identity to SEQ ID NO:15; wherein the OleC homolog comprises a polypeptide having at least 70%, 75%, 80%, 85%, 90% , 92%, 94%, 95%, e.g., 96%, 97%, 98% or 99%, amino acid sequence identity to one of SEQ ID Nos. 17-21; or wherein the OleD homolog comprises a polypeptide having at least 70%, 75%, 80%, 85%, 90%, 92%, 94%, 95%, e.g., 96%, 97%, 98% or 99%, amino acid sequence identity to SEQ ID NO:16, 22, 23 or 24.

12. The method of claim 1 wherein at least one of the OleA, the OleC and the OleD is from a different organism.

13. A method for altering the ratio of trans lactones in a mixture of lactones, comprising: combining mixed diastereomers of an oxetan-2-one with OleA or a homolog thereof, OleD or a homolog thereof and OleC or a homolog thereof, so as to yield a mixture with an altered amount of trans-β-lactones.

14. A method to identify β-lactone synthetase activity, comprising:
combining at room temperature and a pH of about 6 to about 8, a sample suspected of having β-lactone synthetase and a dialkene, a dialkyne or a compound with an alkene and alkene group, so as to yield a mixture; and
detecting in the mixture a change in UV absorbance over time, wherein a change in absorbance is indicative of the presence or amount of a β-lactone synthetase.

15. A host cell comprising a genome augmented with a nucleic acid encoding OleA or a homolog thereof, a nucleic acid encoding OleC or a homolog thereof and a nucleic acid encoding OleD or a homolog, thereof, but which lacks OleB activity, wherein the host cell is heterologous to one or more of the OleA or homolog thereof, the OleC or homolog therof, the OleD or the homolog thereof or a host cell comprising a genome expressing a heterologous OleC.

16. The host cell of claim 15 which is a bacterial cell, a fungal cell or a yeast cell.

17. The host cell of claim 15 wherein the nucleic acid encoding OleA or a homolog thereof, a nucleic acid encoding OleC or a homolog thereof, and a nucleic acid encoding OleD or a homolog thereof are linked.

18. The host cell of claim 15 wherein the host cell has a mutated OleB gene.

19. The host cell of claim 15 wherein at least one of the OleA, the OleC, or the OleD, is heterologous to the host cell or wherein the OleA is heterologous to the OleC or the OleD, the OleC is heterologous to the OleA or the OleD, the OleD is heterologous to the OleC or the OleA, the OleA is heterologous to the OleC and the OleD, the OleC is heterologous to the OleA and the OleD, or the OleD is heterologous to the OleC and the OleA,

20. The method of claim 15 wherein the OleC has at least 70%, 75%. 80%, 85%, 90%, 92%, 94%, 95%, e.g., 96%, 97%, 98% or 99%, amino acid sequence identity to a polypeptide encoded by SEQ ID NO:3 or the OleD has at least 70%, 75%, 80%, 85%, 90%, 92%, 94%, 95%, e.g., 96%, 97%, 98% or 99%, amino acid sequence identity to SEQ ID NO:16, 22, 23 or 24.

21. A method of using the host cell of claim 15, comprising combining the host cell and one or more 3-hydroxy acid substrates, one or more acyl CoA substrates, one or more distinct activated acyl substrates, one or more distinct carboxylic acid substrates, or one or more distinct fatty acid substrates, so as to yield one or more oxetan-2-ones.
